(19) United States
(12) Patent Application Publication
(10) Pub. No.: US 2008/0213833 A1
(43) Pub. Date: Sep. 4, 2008

(54) Methods for Obtaining Optically Active Glycidyl Ethers and Optically Active Vicinal Diols from Racemic Substrates

(75) Inventors: Adriana Leonora Botes, Edgeley (GB); Michel Labuschagne, Pretoria (ZA); Jeanette Lotter, Edenvale Ridge (ZA); Robin Kumar Mitra, Edgeley (GB)

Correspondence Address: FISH & RICHARDSON P.C., P.O. BOX 1022, MINNEAPOLIS, MN, 55440-1022, US

(73) Assignee: Oxrane (UK) Ltd., Manchester (GB)

(21) Appl. No.: 11/872,589

(22) Filed: Oct. 15, 2007

(30) Foreign Application Priority Data
Apr. 15, 2005 (ZA) 2005/03085
Jan. 16, 2006 (IB) PCT/IB2006/050143

(51) Int. Cl. C12P 21/02; C12P 7/18; C12N 9/14; C07H 21/04; C07K 16/00; C07K 14/00; C12N 1/00; C12P 17/02

(52) U.S. Cl. 435/69.1; 435/158; 435/123; 435/71.1; 435/195; 435/243; 536/23.2; 530/350; 530/387.9

(57) ABSTRACT

The invention provides yeast strains, and polypeptides encoded by genes of such yeast strains, that have enantiospecific glycidyl ether hydrolase activity. The invention also features nucleic acid molecules encoding such polypeptides, vectors containing such nucleic acid molecules, and cells containing such vectors. Also embraced by the invention are methods for obtaining optically active glycidyl ethers and associated optically active vicinal diols.

### TECHNICAL FIELD

This invention relates to epoxide hydrolases and biocatalytic reactions using said epoxide hydrolases to produce optically active epoxides and vicinal diols.

### BACKGROUND

Optically active epoxides and vicinal diols are versatile fine chemical intermediates for use in the production of pharmaceuticals, agrochemicals, ferro-electric liquid crystals and flavours and fragrances. Epoxides are highly reactive electrophiles because of the strain inherent in the three-membered ring and the electronegativity of the oxygen. Epoxides react readily with various O-, N-, S-, and C-nucleophiles, acids, bases, reducing and oxidizing agents, allowing access to bifunctional molecules. Vicinal diols, employed as their highly reactive cyclic sulfites and sulfates, act like epoxide-like synthons with a broad range of nucleophiles. The possibility of double nucleophilic displacement reactions with amidines and azide, allow access to dihydroimidazole derivatives, aziridines, diamines and diazides. Since enantiopure epoxides and vicinal diols can be stereospecifically interconverted, they can be regarded as synthetic equivalents. Glycidyl ethers are epoxides of general formula (I).

Optically active glycidyl ethers and their corresponding O¹-substituted glycerols are biologically active compounds and useful synthons in the production of biologically active compounds. For example, guaifenesin (expectorant), mephenesin (muscle relaxant) and chlorphenesin (antifungal) are aryloxy diols in which the desired biological activity resides in the (S)-enantiomers, (S)-Aryl glycidyl ethers are useful synthons for β-adrenergic receptor blocking agents (β-blockers).

Epoxide hydrolases (EC 3.3.2.3) are hydrolytic enzymes that convert epoxides to vicinal diols by ring-opening of the epoxide with water. Epoxide hydrolases are present in mammals, plants, insects and microorganisms.

### SUMMARY

The invention is based in part on the surprising discovery by the inventors that certain microorganisms express epoxide hydrolases which act on glycidyl ether substrates with high enantioselectivity. These microorganisms and the associated enantioselective glycidyl ether hydrolase (YEGH) polypeptides of the invention selectively hydrolyse specific enantiomers of a range of different glycidyl ethers (GE). Genomes of the microorganisms therefore encode polypeptides having highly enantioselective glycidyl ether hydrolase activity.

More specifically, the invention provides a process for obtaining an optically active glycidyl ether and/or an optically active vicinal diol, which process includes the steps of: providing an enantiomeric mixture of a glycidyl ether (GE); creating a reaction mixture by adding to the enantiomeric mixture a polypeptide, or a functional fragment thereof, having enantioselective glycidyl ether hydrolase (YEGH) activity, the polypeptide being a polypeptide encoded by a gene of a yeast cell or a gene derived from a yeast cell; incubating the reaction mixture; and recovering from the reaction mixture: at least one of an enantiopure, or a substantially enantiopure vicinal diol (GD), and an enantiopure, or a substantially enantiopure, glycidyl ether (GE).

According to another aspect of the invention there is provided a process for obtaining an optically active glycidyl ether and/or an optically active vicinal diol, which process includes the steps of: providing an enantiomeric mixture of a glycidyl ether (GE); creating a reaction mixture by adding to the enantiomeric mixture a cell comprising a nucleic acid encoding, and capable of expressing, a polypeptide having enantioselective glycidyl ether hydrolase (YEGH) activity, the polypeptide being a polypeptide encoded by a gene of a yeast cell; incubating the reaction mixture; and recovering from the reaction mixture: at least one of an enantiopure, or a substantially enantiopure, vicinal diol (GD), and an enantiopure, or a substantially enantiopure, glycidyl ether (GE).

In both of the above processes, the incubation may result in the selective production of a GD having the chirality of the enantiomer for which the epoxide hydrolase has selective activity and/or the selective enrichment, relative to the total amount of both enantiomers of the GE in the mixture, of the GE enantiomers for which the epoxide hydrolase does not have selective activity.

The following embodiments apply to both of the above processes. The cell can be a yeast cell. The polypeptide can be encoded by an endogenous gene of the cell or the cell can be a recombinant cell, the polypeptide being encoded by a nucleic acid sequence with which the cell is transformed. The nucleic acid sequence can be an exogenous nucleic acid sequence, a heterologous nucleic acid sequence, or a homologous nucleic acid sequence. The polypeptide can be a full-length yeast epoxide hydrolase or a functional fragment of a full length yeast epoxide hydrolase.

Moreover both processes can be carried out at a pH from 5 to 10. They can be carried out at a temperature of 0° C. to 70° C. In the processes, the concentration of the glycidyl ether can be at least equal to the solubility of the GE in water.

In both processes, the glycidyl ether (GE) is a compound of the general formula (I) and the vicinal diol (GD) produced by the process is a compound of the general formula (II),

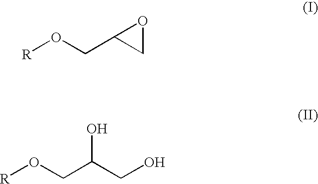

wherein,

R represents a variably substituted straight-chain or branched alkyl group, a variably substituted straight-chain or branched alkenyl group, a variably substituted straight-chain or branched alkynyl group, a variably substituted cycloalkyl group as well as cycloalkenyl groups, a variably substituted aryl group, a variably substituted aryl alkyl group, a variably substituted heterocyclic group, a variably substituted alkylthio group, a variably substituted alkoxycarbonyl group, a variably substituted straight chain or branched alkylamino or alkenyl amino group, a variably substituted arylamino or arylalkylamino group, a variably substituted carbamoyl group, or a variably substituted acyl group.

R can also take the form of R′—X, where X is a functional group bonded to any C of R′ except C₁.

—OR as a whole can also be replaced by a functional group.

The alkyl group may be a straight chain or branched alkyl group with 1 to 12 carbon atoms but preferably the alkyl group is as straight chain or branched alkyl group with 1 to 8 carbons.

The alkenyl group may be a straight chain or branched alkenyl group having 2-12 carbon atoms but preferably the alkenyl group is a straight chain or branched alkenyl group with 2 to 8 carbons.

The alkynyl group may be a straight chain or branched alkynyl group having 2-12 carbon atoms but preferably the alkynyl group is a straight chain or branched alkenyl group with 2 to 8 carbons.

The cycloalkyl group may include cycloalkyl groups with 3 to 10 carbon atoms. Examples include the cyclopropyl-, cyclobutyl-, cyclopentyl-, cyclohexyl-, cycloheptyl- and cyclooctyl-groups that may be variably substituted at any position(s) around the ring. Preferably the cycloalkyl group is a cycloalkyl group with 5 to 7 carbon atoms.

The cycloalkenyl group may include cycloalkenyl groups with 3 to 10 carbon atoms. Examples include cyclobutenyl-, cyclopentenyl-, cyclohexenyl-, cycloheptenyl- and cyclooctenyl-groups that may be variably substituted at any position(s) around the ring. Preferably the cycloalkenyl group is a cycloalkenyl group with 5 to 7 carbon atoms.

The aryl group may include phenyl, biphenyl, naphtyl, anthracenyl groups and the like. Preferably the aryl group is a phenyl group. The aryl alkyl group may include a group with 7 to 18 carbons, but preferably the aryl alkyl group is an aryl alkyl group with 7 to 12 carbon atoms.

The heterocyclic group may include 5- to 7-membered heterocyclic groups containing nitrogen, oxygen or sulfur. The heterocyclic ring may be fused with a cyclic or aromatic ring having 3 to 7 carbon atoms such as a benzene, cyclopropyl, cyclobutane, cyclopentane and cyclohexane ring systems. A ring with 5 or 6 carbon atoms is preferred.

The alkylamino group may include a straight chain or branched alkylamino group having 2-12 carbon atoms such as methylamino, ethylamino, propylamino, isopropylamino, butylamino, isobutylamino, tert-butylamino, pentylamino, hexylamino, heptylamino or octylamino.

The alkenyl amino group may include a straight chain or branched alkenylamino group having 2-12 carbon atoms but preferably the alkenyl amino group is a straight chain or branched alkenylamino group with 2 to 8 carbons.

The arylamino group may include arylamino groups such as a phenylamino or naphtylamino group which may be optionally substituted with an alkyl or alkenyl or alkoxy group having 1 to 4 carbon atoms, and also halogens, The arylalkylamino group may include benzylamino and 2-phenylethylamino.

The alkylthio group may include alkylthio groups having 1 to 8 carbon atoms such as methylthio, ethylthio, propylthio, butylthio, isobutylthio, pentylthio.

The alkenylthio group may include a straight chain or branched alkenylthio group having 1 to 8 carbon atoms such as ethynylthio-, 1-propynylthio-, 2-propynylthio-, 1-butynylthio-, 2-butynylthio-, 3-butynylthio-, 1-pentynylathio-, 2-pentynylthio-, 3-pentynylthio-, 4-pentynylthio-, 1-hexynylthio-, 2-hexynylthio-, 3-hexynylthio-, 4-hexynylthio-, 5-hexynylthio- and the like.

The arylthio group may include alkenylthio groups having 1 to 8 carbon atoms such as a phenylthio or naphtylthio group which may be optionally substituted with an alkyl or alkenyl or alkoxy group having 1 to 4 carbon atoms, and also halogens, e.g. phenylthio, 2-methylphenylthio, 3-methylphenylthio, 4-methylphenylthio, 2-allylphenylthio, 2-chlorophenylthio, 3-chlorophenylamini, 4-chlorophenylthio, 4-methoxyphenylthio, 2-allyloxyphenylthio, naphtylthio and the like.

The arylalkylthio group may include alkenylthio groups having 1 to 8 carbon atoms such as the benzylthio-group and 2-phenylethylthio-group.

The alkoxycarbonyl group may include methoxycarbonyl, ethoxycarbonyl and the like.

The substituted or unsubstituted carbamoyl group may include carbamoyl, methylcarbamoyl, dimethylcarbamoyl, diethylcarbamoyl and the like.

The acyl group may include acyl groups with 1 to 8 carbon atoms such as formyl, acetyl, propionyl or benzoyl groups and others.

The alkyl, alkenyl, alkynyl, cycloalkyl, aryl, aryl alkyl, heterocyclic, alkylamino, alkenylamino, arylamino, arylalkylamino, alkylthio, alkenylthio, arylthio, arylalkylthio, alkoxycarbonyl, substituted and unsubstituted carbamoyl and acyl groups mentioned above may optionally be substituted. Examples of such substituents include halogens (F, Cl, Br, I), hydroxyl groups, mercapto groups, carboxylates, nitro groups, cyano groups, substituted or unsubstituted amino groups (including amino, methylamino, dimethylamino, ethylamino, diethylamino, and various protected amines such as tert-butoxycarbonyl- and arylsulfonamido groups), alkoxy groups (having 1 to 8 carbon atoms such as methoxy, ethoxy, propyloxy, isopropyloxy, butyloxy, isobutyloxy, tert-butyloxy, pentyloxy, hexyloxy, heptyloxy or octyloxy), alkenyloxy groups (having 2 to 8 carbon atoms such as a vinyloxy, allyloxy, 3-butenyloxy or 5-hexenyloxy), aryloxy groups (such as a phenoxy or naphtyloxy group which may be optionally substituted with an alkyl or alkenyl or alkoxy group having 1 to 4 carbon atoms, and also halogens, e.g. phenoxy, 2-methylphenoxy, 3-methylphenoxy, 4-methylphenoxy, 2-allylphenoxy, 2-chlorophenoxy, 3-chlorophenoxy, 4-chlorophenoxy, 4-methoxyphenoxy, 2-allyloxyphenoxy, naphtyloxy and the like), aryl alkyloxy groups (e.g. benzyloxy and 2-phenylethyloxy), alkylthio groups (having 1 to 8 carbon atoms such as methylthio, ethylthio, propylthio, butylthio, isobutylthio, pentylthio), alkoxycarbonyl groups (e.g. methoxycarbonyl, ethoxycarbonyl and the like), substituted or unsubstituted carbamoyl group (e.g. carbamoyl, methylcarbamoyl, dimethylcarbamoyl, diethylcarbamoyl and the like), acyl groups (with 1 to 8 carbon atoms such as formyl, acetyl, propionyl or benzoyl groups) and others.

The above-mentioned cycloalkyl, cycloalkenyl, aryl, aryl alkyl, heterocyclic, alkoxy, alkenyloxy, aryloxy, aryl alkyloxy, alkylthio, and alkoxycarbonyl groups may also be substituted with alkyl groups having 1 to 5 carbon atoms, alkenyl groups with 2 to 5 carbon atoms, or haloalkyl groups with 1 to 5 carbon atoms in addition to the substituents specified above.

The number of substituents may be one or more than one.

The substituents may be the same or different.

R can also take the form of R′—X, where X is a functional group bonded to any carbon of R′ except C₁. The functional group may be for example a halogen (F, Cl, Br, I), hydroxyl group, mercapto group, carboxylate, nitro group, cyano group, substituted or unsubstituted amino group (including amino, methylamino, dimethylamino, ethylamino, diethylamino), and various protected amines such as a tert-butoxycarbonyl- or a arylsulfonamido group

—OR as a whole can also be replaced by a functional group such as a halogen (F, Cl, Br, I), hydroxyl group, mercapto group, carboxylate, nitro group, cyano group, substituted or unsubstituted amino group (including amino, methylamino, dimethylamino, ethylamino, diethylamino), and various protected amines such as a tert-butoxycarbonyl- or a arylsulfonamido group.

Moreover, in the processes, the enantiomeric mixture can be a racemic mixture or a mixture of any ratio of amounts of the enantiomers. The processes can include adding to the reaction mixture water and at least one water-immiscible solvent, including, for example, toluene, 1,1,2-trichlorotrifluoroethane, methyl tert-butyl ether, methyl isobutyl ketone, dibutyl-o-phtalate, aliphatic alcohols containing 6 to 9 carbon atoms or aliphatic hydrocarbons containing 6 to 16 carbon atoms.

Alternatively, or in addition, the processes can include adding to the reaction mixture water and at least one water-miscible organic solvent, for example, acetone, methanol, ethanol, propanol, isopropanol, acetonitrile, dimethylsulfoxide, N,N-dimethylformamide, or N-methylpyrrolidine. In addition, or alternatively, one or more surfactants, one or more cyclodextrins, or one or more phase-transfer catalysts can be added to the reaction mixtures. Both processes can include stopping the reaction when one enantiomer of a GE and/or associated GD is in excess compared to the other enantiomer of the GE and/or GD. Furthermore, the processes can include directly recovering continuously from the reaction mixture during the reaction an optically active GE and/or associated optically active GD produced by the reaction.

In both processes the yeast cell can be of one of the following exemplary genera: Arxula, Brettanomyces, Bullera, Bulleromyces, Candida, Cryptococcus, Debaryomyces, Dekkera, Exophiala, Geotrichum, Hormonema, Issatchenkia, Kluyveromyces, Lipomyces, Mastigomyces, Myxozyma, Pichia, Rhodosporidium, Rhodotorula, Sporidiobolus, Sporobolomyces, Trichosporon, Wingea, and Yarrowia.

Moreover, in the processes, the yeast cell can be of one of the following exemplary species: Arxula adeninivorans, Arxula terrestris, Brettanomyces bruxellensis, Brettanomyces naardenensis, Brettanomyces anomalus, Brettanomyces species (e.g. Unidentified species NCYC 3151), Bullera dendrophila, Bulleromyces albus, Candida albicans, Candida fabianii, Candida glabrata, Candida haemulonii, Candida intermedia, Candida magnoliae, Candida parapsilosis, Candida rugosa, Candida tenuis, Candida tropicalis, Candida famata, Candida kruisei, Candida sp. (new) related to C. sorbophila, Cryptococcus albidus, Cryptococcus amylolentus, Cryptococcus bhutanensis, Cryptococcus curvatus, Cryptococcus gastricus, Cryptococcus humicola, Cryptococcus hungaricus, Cryptococcus laurentii, Cryptococcus luteolus, Cryptococcus macerans, Cryptococcus podzolicus, Cryptococcus terreus, Debaryomyces hansenii, Dekkera anomala, Exophiala dermatitidis, Geotrichum spp. (e.g. Unidentified species UOFS Y-0111), Hormonema spp. (e.g. Unidentified species NCYC 3171), Issatchenkia occidentalis, Kluyveromyces marxianus, Lipomyces spp. (e.g. Unidentified species UOFS Y-2159), Lipomyces tetrasporus, Mastigomyces philipporii, Myxozyma melibiosi, Pichia anomala, Pichia finlandica, Pichia guillermondii, Pichia haplophila, Rhodosporidium lusitaniae, Rhodosporidium paludigenum, Rhodosporidium sphaerocarpum, Rhodosporidium toruloides, Rhodosporidium paludigenum, Rhodotorula araucariae, Rhodotorula glutinis, Rhodotorula minuta, Rhodotorula minuta var. minuta, Rhodotorula mucilaginosa, Rhodotorula philyla, Rhodotorula rubra, Rhodotorula species (e.g. Unidentified species NCYC 3193, UOFS Y-2042, UOFS Y-0448, UOFS Y-0139, UOFS Y-0560), Rhodotorula aurantiaca, Rhodotorula spp. (e.g. Unidentified species NCYC 3224), Rhodotorula sp. “mucilaginosa”, Sporidiobolus salmonicolor, Sporobolomyces holsaticus, Sporobolomyces roseus, Sporobolomyces tsugae, Trichosporon beigelii, Trichosporon cutaneum var. cutaneum, Trichosporon delbrueckii, Trichosporon jirovecii, Trichosporon mucoides, Trichosporon ovoides, Trichosporon pullulans, Trichosporon spp. (e.g. Unidentified species NCYC 3210, NCYC 3211, UOFS Y-0861, UOFS Y-1615, UOFS Y-0451, NCYC 3212, UOFS Y-0449, UOFS Y-2113), Trichosporon moniliiforme, Trichosporon montevideense, Wingea robertsiae, and Yarrowia lipolytica.

The yeast cell can also be of any of the other genera, species, or strains disclosed herein.

Another aspect of the invention is a method for producing a polypeptide, which process includes the steps of: providing a cell comprising a nucleic acid encoding and capable of expressing a polypeptide that has enantioselective glycidyl ether hydrolase (YEGH) activity; culturing the cell; and recovering the polypeptide from the culture. Recovering the polypeptide from the culture includes, for example, recovering it from the medium in which the cells were cultured or recovering it from the cell per se. The cell can be a yeast cell. The polypeptide can be encoded by an endogenous gene of the cell or the cell can be a recombinant cell, the polypeptide being encoded by a nucleic acid sequence with which the cell is transformed. The nucleic acid sequence can be an exogenous nucleic acid sequence, a heterologous nucleic acid sequence, or a homologous nucleic acid sequence. The polypeptide can be a full-length yeast epoxide hydrolase or a functional fragment of a full-length yeast epoxide hydrolase. The cell can be of any of the yeast genera, species, or strains disclosed herein or any recombinant cell disclosed herein.

The invention also features a crude or pure enzyme preparation which includes an isolated polypeptide having YEGH activity. The polypeptide can be one encoded by any of the yeast genera, species, or strains disclosed herein or one encoded by a recombinant cell.

In another aspect, the invention features a substantially pure culture of cells, a substantial number of which comprise a nucleic acid encoding, and are capable of expressing, a polypeptide having YEGH activity. The cells can be recombinant cells or cells of any of the yeast genera, species, or strains disclosed herein.

Another embodiment of the invention is an isolated cell, the cell comprising a nucleic acid encoding a polypeptide having YEGH activity, the cell being capable of expressing the polypeptide. The cell can be any of those disclosed herein.

The invention also features an isolated DNA that includes: (a) a nucleic acid sequence that encodes a polypeptide that has YEGH activity and that hybridizes under highly stringent conditions to the complement of a sequence that can be SEQ ID NO: 10, 11, 12, 13, 14, 15, 16, 17, or 18; or (b) the complement of the nucleic acid sequence. The nucleic acid sequence can encode a polypeptide that includes an amino acid sequence that can be SEQ ID NO: 1, 2, 3, 4, 5, 6, 7, 8, or 9. The nucleic acid sequence can be, for example, one of those with SEQ ID NOs: 10, 11, 12, 13, 14, 15, 16, 17, or 18.

Also provided by the invention is an isolated DNA that includes: (a) a nucleic acid sequence that is at least 55% identical to a sequence that can be SEQ ID NO: 10, 11, 12, 13, 14, 15, 16, 17, or 18; or (b) the complement of the nucleic acid sequence, the nucleic acid sequence encoding a polypeptide that has YEGH activity.

Another aspect of the invention is an isolated DNA that includes: (a) a nucleic acid sequence that encodes a polypeptide consisting of an amino acid sequence that is at least 55% identical to a sequence that can be SEQ ID NOs: 1, 2, 3, 4, 5, 6, 7, 8, or 9; or (b) the complement of the nucleic acid sequence, the polypeptide having YEGH activity. Also included are vectors (e.g., those in which the coding sequence is operably linked to a transcriptional regulatory element) containing any of the above DNAs and cells (e.g., eukaryotic or prokaryotic cells) containing such vectors.

Also provided by the invention is an isolated polypeptide encoded by any of the above DNAs. The polypeptide can include an amino acid sequence that is at least 55% identical to SEQ ID NOs: 1, 2, 3, 4, 5, 6, 7, 8, or 9, the polypeptide having YEGH activity. The polypeptide can also include: (a) a sequence that can be SEQ ID NO: 1, 2, 3, 4, 5, 6, 7, 8, or 9, or a functional fragment of the sequence; or (b) the sequence of (a), but with no more than five conservative substitutions, the polypeptide having YEGH activity.

In another embodiment the invention features an isolated antibody (e.g., a polyclonal or a monoclonal antibody) that binds to any of the above-described polypeptides.

The term “exogenous” as used herein with reference to nucleic acid and a particular host cell refers to any nucleic acid that does not occur in (and cannot be obtained from) that particular cell as found in nature. Thus, a non-naturally-occurring nucleic acid is considered to be exogenous to a host cell once introduced into the host cell. It is important to note that non-naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host cell, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. Nucleic acid that is naturally-occurring can be exogenous to a particular cell. For example, an entire chromosome isolated from a cell of yeast x is an exogenous nucleic acid with respect to a cell of yeast y once that chromosome is introduced into a cell of yeast y.

It will be clear from the above that “exogenous” nucleic acids can be “homologous” or “heterologous” nucleic acids. As used herein, “homologous” nucleic acids are those that are derived from a cell of the same species as the host cell and “heterologous” nucleic acids are those that are derived from a species other than that of the host cell. In contrast, the term “endogenous” as used herein with reference to nucleic acids or genes and a particular cell refers to any nucleic acid or gene that does occur in (and can be obtained from) that particular cell as found in nature.

The glycidyl ether used by the methods of the invention may be a compound of the general formula (I) and the vicinal diol produced by the process may be a compound of the general formula (II),

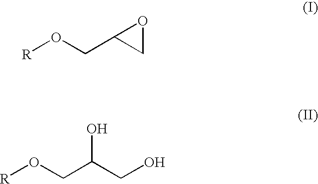

### Wherein;

R represents a variably substituted straight-chain or branched alkyl group, a variably substituted straight-chain or branched alkenyl group, a variably substituted straight-chain or branched alkynyl group, a variably substituted cycloalkyl group as well as cycloalkenyl groups, a variably substituted aryl group, a variably substituted aryl alkyl group, a variably substituted heterocyclic group, a variably substituted alkylthio group, a variably substituted alkoxycarbonyl group, a variably substituted straight chain or branched alkylamino or alkenyl amino group, a variably substituted arylamino or arylalkylamino group, a variably substituted carbamoyl group, or a variably substituted acyl group.

R can also take the form of R′—X, where X is a functional group bonded to any C of R′ except C₁.

—OR as a whole can also be replaced by a functional group.

The alkyl group may be a straight chain or branched alkyl group with 1 to 12 carbon atoms. Examples include the methyl-, ethyl-, propyl-, isopropyl-, butyl-, isobutyl-, s-butyl-, t-butyl-, pent-1-yl-, pent-2-yl-, pent-3-yl-, 2-methylbut-1-yl-, 3-methylbut-1-yl-, 2-methylbut-2-yl-, 3-methylbut-2-yl-, hex-1-yl-, hex-2-yl-, hex-3-yl-, 1-methylpent-1-yl-, 2-methylpent-1-yl-, 3-methylpent-1-yl-, 2-methylpent-2-yl-, 3-methylpent-2-yl-, 4-methylpent-2-yl-, 2-methylpent-3-yl-, 3-methylpent-3-yl-, 2-ethylbut-1-yl-, hept-1-yl-, hept-2-yl-, hept-3-yl-, hept-4-yl-, 1-methylhex-1-yl-, 2-methylhex-1-yl-, 3-methylhex-1-yl-, 4-methylhex-1-yl-, 5-methylhex-1-yl-, 2-methylhex-2-yl-, 3-methylhex-2-yl-, 4-methylhex-2-yl-, 5-methylhex-2-yl-, 2-methylhex-3-yl-, 3-methylhex-3-yl-, 4-methylhex-3-yl-, 5-methylhex-3-yl-, 2-methylhex-4-yl-, 1,1-dimethylpent-1-yl-, 1,2-dimethylpent-1-yl-, 1,3-dimethylpent-1-yl-, 1,4-dimethylpent-1-yl-, 2,2-dimethylpent-1-yl-, 2,3-dimethylpent-1-yl-, 2,4-dimethylpent-1-yl-, 2,5-dimethylpent-1-yl-, 3,3-dimethylpent-1-yl-, 3,4-dimethylpent-1-yl-, 3,5-dimethylpent-1-yl-, 4,4-dimethylpent-1-yl-, 4,5-dimethylpent-1-yl-, 5,5-dimethylpent-1-yl-, 2,2-dimethylpent-2-yl-, 2,3-dimethylpent-2-yl-, 2,4-dimethylpent-2-yl-, 3,3-dimethylpent-2-yl-, 3,4-dimethylpent-2-yl-, 2,2-dimethylpent-3-yl-, 2,3-dimethylpent-3-yl-, 2,4-dimethylpent-3-yl-, 2,2-dimethylpent-4-yl-, 2-ethylpent-1-yl-, 3-ethylpent-1-yl-, 1,1,2-trimethylbut-1-yl-, 1,2,2-trimethylbut-1-yl-, 1,2,3-trimethylbut-1-yl-, 2,2,3-trimethylbut-1-yl-, 2,3,3-trimethylbut-1-yl-, 2,3,3-but-2-yl-, 2-isopropylbut-1-yl-, 2-isopropylbut-2-yl-, oct-1-yl-, oct-2-yl-, oct-3-yl-, oct-4-yl-, 2-methylhept-1-yl-, 3-methylhept-1-yl-, 4-methylhept-1-yl-, 5-methylhept-1-yl-, 6-methylhept-1-yl-, 2-methylhept-2-yl-, 3-methylhept-2-yl-, 4-methylhept-2-yl-, 5-methylhept-2-yl-, 6-methylhept-2-yl-, 2-methylhept-3-yl-, 3-methylhept-3-yl-, 4-methylhept-3-yl-, 5-methylhept-3-yl-, 6-methylhept-3-yl-, 2-methylhept-4-yl-, 3-methylhept-4-yl-, 4-methylhept-4-yl-, 2,2-dimethylhex-1-yl-, 2,3-dimethylhex-1-yl-, 2,4-dimethylhex-1-yl-, 2,5-dimethylhex-1-yl-, 3,3-dimethylhex-1-yl-, 3,4-dimethylhex-1-yl-, 3,5-dimethylhex-1-yl-, 4,4-dimethylhex-1-yl-, 4,5-dimethylhex-1-yl-, 5,5-dimethylhex-1-yl-, 2,3-dimethylhex-2-yl-, 2,4-dimethylhex-2-yl-, 2,5-dimethylhex-2-yl-, 3,3-dimethylhex-2-yl-, 3,4-dimethylhex-2-yl-, 3,5-dimethylhex-2-yl-, 4,4-dimethylhex-2-yl-, 4,5-dimethylhex-2-yl-, 5,5-dimethylhex-2-yl-, 2,2-dimethylhex-3-yl-, 2,3-dimethylhex-3-yl-, 2,4-dimethylhex-3-yl-, 2,5-dimethylhex-3-yl-, 3,3-dimethylhex-3-yl-, 3,4-dimethylhex-3-yl-, 3,5-dimethylhex-3-yl-, 4,4-dimethylhex-3-yl-, 4,5-dimethylhex-3-yl-, 5,5-dimethylhex-3-yl-, 2,2,3-trimethylpent-1-yl-, 2,2,4-trimethylpent-1-yl-, 2,3,3-trimethylpent-1-yl-, 2,3,4-trimethylpent-1-yl-, 3,3,4-trimethylpent-1-yl-, 3,4,4-trimethylpent-1-yl-, 2,4,4-trimethylpent-1-yl-, 2,3,3-trimethylpent-2-yl-, 2,3,4-trimethylpent-2-yl-, 3,3,4-trimethylpent-2-yl-, 3,4,4-trimethylpent-2-yl-, 2,4,4-trimethylpent-2-yl-, 2,2,3-trimethylpent-3-yl-, 2-methyl-3-ethylpen-1-yl-, 3-ethyl-3-methylpent-1-yl-, 3-ethyl-4-methylpent-1-yl-, (3-methylhex-3-yl)methyl-, (4-methylhex-3-yl)methyl-, (5-methylhex-3-yl)methyl-, (2-methylhex-2-yl)methyl-, 2-methyl-3-ethylpent-2-yl-, 3-ethyl-3-methylpent-2-yl-, 3-ethyl-4-methylpent-2-yl-, 2-methyl-2-ethylpent-3-yl-, 2-methyl-3-ethylpent-3-yl-, 2,2,3,3-tetramethylbut-1-yl-, 2-ethyl-3,3-dimethylbut-2-ly, 2-isopropyl-3-methylbut-2-yl-, (3-ethyl pent-3-yl)methyl-, (2,3-dimethylpent-3-yl)methyl-, (2,4-dimethylpent-3-yl)methyl-, non-1-yl-, non-2-yl-, non-3-yl-, non-4-yl-, non-5-yl-, 2-methyloct-1-yl, 3-methyloct-1-yl-, 4-methyloct-1-yl-, 5-methyloct-1-yl-, 6-methyloct-1-yl-, 7-methyloct-1-yl-, 2-methyloct-2-yl, 3-methyloct-2-yl-, 4-methyloct-2-yl-, 5-methyloct-2-yl-, 6-methyloct-2-yl-, 7-methyloct-2-yl-, 2-methyloct-3-yl, 3-methyloct-3-yl-, 4-methyloct-3-yl-, 5-methyloct-3-yl-, 6-methyloct-3-yl-, 7-methyloct-3-yl-, 2-methyloct-4-yl, 3-methyloct-4-yl-, 4-methyloct-4-yl-, 5-methyloct-4-yl-, 6-methyloct-4-yl-, 7-methyloct-4-yl-, 2,2-dimethylhept-1-yl-, 2,3-dimethylhept-1-yl-, 2,4-dimethylhept-1-yl-, 2,5-dimethylhept-1-yl-, 2,6-dimethylhept-1-yl-, 3,3-dimethylhept-1-yl-, 3,4-dimethylhept-1-yl-, 3,5-dimethylhept-1-yl-, 3,6-dimethylhept-1-yl-, 4,4-dimethylhept-1-yl-, 4,5-dimethylhept-1-yl-, 4,6-dimethylhept-1-yl-, 5,5-dimethylhept-1-yl-, 5,6-dimethylhept-1-yl-, 6,6-dimethylhept-1-yl-, 2,3-dimethylhept-2-yl-, 2,4-dimethylhept-2-yl-, 2,5-dimethylhept-2-yl-, 2,6-dimethylhept-2-yl-, 3,3-dimethylhept-2-yl-, 3,4-dimethylhept-2-yl-, 3,5-dimethylhept-2-yl-, 3,6-dimethylhept-2-yl-, 4,4-dimethylhept-2-yl-, 4,5-dimethylhept-2-yl-, 4,6-dimethylhept-2-yl-, 5,5-dimethylhept-2-yl-, 5,6-dimethylhept-2-yl-, 6,6-dimethylhept-2-yl-, 2,2-dimethylhept-3-yl-, 2,3-dimethylhept-3-yl-, 2,4-dimethylhept-3-yl-, 2,5-dimethylhept-3-yl-, 2,6-dimethylhept-3-yl-, 3,4-dimethylhept-3-yl-, 3,5-dimethylhept-3-yl-, 3,6-dimethylhept-3-yl-, 4,4-dimethylhept-3-yl-, 4,5-dimethylhept-3-yl-, 4,6-dimethylhept-3-yl-, 5,5-dimethylhept-3-yl-, 5,6-dimethylhept-3-yl-, 6,6-dimethylhept-3-yl-, 3-ethylhept-1-yl-, 3-ethylhept-1-yl-, 4-ethylhept-1-yl-, 3-ethylhept-2-yl-, 4-ethyl hept-2-yl-, 5-ethyl hept-2-yl-, 3-ethyl hept-3-yl-, 4-ethyl hept-3-yl-, 5-ethylhept-3-yl-, 3-ethylhept-4-yl-, 4-ethylhept-4-yl-, 2,2,3-trimethylhex-1-yl-, 2,2,4-trimethylhex-1-yl-, 2,2,5-trimethylhex-1-yl-, 2,3,3-trimethylhex-1-yl-, 2,3,4-trimethylhex-1-yl-, 2,3,5-trimethylhex-1-yl-, 2,4,4-trimethylhex-1-yl-, 2,4,5-trimethylhex-1-yl-, 2,5,5-trimethylhex-1-yl-, 3,3,4-trimethylhex-1-yl-, 3,3,5-trimethylhex-1-yl-, 4,4,5-trimethylhex-1-yl-, 4,5,5-trimethylhex-1-yl-, 2,3,3-trimethylhex-2-yl-, 2,3,4-trimethylhex-2-yl-, 2,3,5-trimethylhex-2-yl-, 2,4,4-trimethylhex-2-yl-, 2,4,5-trimethylhex-2-yl-, 2,5,5-trimethylhex-2-yl-, 3,3,4-trimethylhex-2-yl-, 3,3,5-trimethylhex-2-yl-, 3,4,4-trimethylhex-2-yl-, 3,4,5-trimethylhex-2-yl-, 3,5,5-trimethylhex-2-yl-, 4,4,5-trimethylhex-2-yl-, 4,5,5-trimethylhex-2-yl-, 2,2,3-trimethylhex-3-yl-, 2,2,4-trimethylhex-3-yl-, 2,2,5-trimethylhex-3-yl-, 2,3,4-trimethylhex-3-yl-, 2,3,5-trimethylhex-3-yl-, 2,4,4-trimethylhex-3-yl-, 2,4,5-trimethylhex-3-yl-, 2,5,5-trimethylhex-3-yl, 4,4,5-trimethylhex-3-yl-, 4,5,5-trimethylhex-3-yl-, (2-methylhex-3-yl)methyl-, 3-ethyl-2-methylhex-1-yl-, 3-ethyl-3-methylhex-1-yl-, 3-ethyl-4-methylhex-1-yl-, 3-ethyl-5-methylhex-1-yl-, 4-ethyl-2-methylhex-1-yl-, 4-ethyl-3-methylhex-1-yl-, 4-ethyl-4-methylhex-1-yl-, 4-ethyl-5-methylhex-1-yl-, (2-methylhex-1-yl)methyl-, (3-methylhex-1-yl)methyl-, (4-methylhex-1-yl)methyl-, (5-methylhex-1-yl)methyl-, (6-methylhex-1-yl)methyl-, 3-isopropylhex-1-yl-, 4-ethyl-5-methylhex-1-yl-, 3-ethyl-3-methylhex-2-yl-, 3-ethyl-4-methylhex-2-yl-, 3-ethyl-5-methylhex-2-yl-, 4-ethyl-2-methylhex-2-yl-, 4-ethyl-3-methylhex-2-yl-, 4-ethyl-4-methylhex-2-yl-, 4-ethyl-5-methylhex-2-yl-, 3-isopropylhex-2-yl-, 4-ethyl-5-methylhex-2-yl-, 3-ethyl-2-methylhex-3-yl-, 3-ethyl-4-methylhex-3-yl-, 3-ethyl-5-methylhex-3-yl-, 4-ethyl-2-methylhex-3-yl-, 4-ethyl-3-methylhex-3-yl-, 4-ethyl-4-methylhex-3-yl-, 4-ethyl-5-methylhex-3-yl-, 4-isopropylhex-1-yl-, 2,2,3,3-tetramethylpent-1-yl-, 2,2,3,4-tetramethylpent-1-yl-, 2,2,4,4-tetramethylpent-1-yl-, 2,3,3,4-tetramethylpent-1-yl-, 2,3,4,4-tetramethylpent-1-yl-, 2,3,4,4-tetramethylpent-1-yl-, 3,3,4,4-tetramethylpent-1-yl-, 2,3,3,4-tetramethylpent-2-yl-, 2,3,4,4-tetramethylpent-2-yl-, 2,3,4,4-tetramethylpent-2-yl-, 3,3,4,4-tetramethylpent-2-yl-, 2,2,3,4-tetramethylpent-3-yl-, 2,2,4,4-tetramethylpent-3-yl-, 2,3,4,4-tetramethylpent-3-yl-, 2,3,4,4-tetramethylpent-3-yl-, (3-ethylhex-3-yl)methyl-, (4-ethylhex-3-yl)methyl-, (5-methylhept-3-yl)methyl-, 2,4-dimethyl-3-ethylpent-1-yl-, 3,4-dimethyl-3-ethylpent-1-yl-, 4,4-dimethyl-3-ethylpent-1-yl-, 2-ethyl-2-methylhex-1-yl-, 3-ethyl-2-methylhex-1-yl-, 4-ethyl-2-methylhex-1-yl-, 2-ethyl-3-methylhex-1-yl-, 2-ethyl-4-methylhex-1-yl-, 3-ethyl-3-methylhex-1-yl-, 3-ethyl-4-methylhex-1-yl-, 3-ethyl-5-methylhex-1-yl-, 4-ethyl-3-methylhex-1-yl-, 4-ethyl-4-methylhex-1-yl-, 4-ethyl-5-methylhex-1-yl-, and the like from dec-1-yl-, dec-2-yl-, dec-3-yl-, dec-4-yl-, dec-5-yl-, dec-6-yl-, undec-1-yl-, undec-2-yl-, undec-3-yl-, undec-4-yl-, undec-5-yl-, undec-6-yl-, undec-7-yl-, dodec-1-yl, dodec-2-yl, dodec-3-yl, dodec-4-yl, dodec-5-yl, dodec-6-yl groups.

Preferably the alkyl group is as straight chain or branched alkyl group with 1 to 8 carbons.

The alkenyl group may be a straight chain or branched alkenyl group having 2-12 carbon atoms. Examples include vinyl-, allyl-, α-methallyl-, β-methallyl-, 1-propenyl-, isopropenyl-, 1-butenyl-, 2-butenyl-, 3-butenyl, 1-buten-2-yl-, 1-buten-3-yl-, 1-methyl-1-propenyl-, 2-methyl-1-propenyl-, 1-pentenyl-, 2-pentenyl-, 3-pentenyl-, 4-pentenyl-, 1-penten-2-yl-, 1-penten-3-yl-, 2-methyl-1-butenyl-, 1-hexenyl-, 2-hexenyl-, 3-hexenyl-, 4-hexenyl-, 5-hexenyl-, 1-heptenyl-, 2-heptenyl-, 3-heptenyl-, 4-heptenyl-, 5-heptenyl-, 6-heptenyl-, 1-octenyl-, 2-octenyl-, 3-octenyl-, 4-octenyl-, 5-octenyl-, 6-octenyl-, 7-octenyl-, 1-nonenyl-, 2-nonenyl-, 3-nonenyl-, 4-nonenyl-, 5-nonenyl-, 6-nonenyl-, 7-nonenyl-, 8-nonenyl-, 1-decenyl-, 2-decenyl-, 3-decenyl-, 4-decenyl-, 5-decenyl-, 6-decenyl-, 7-decenyl-, 8-decenyl-, 9-decenyl-, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl and 11-dodecenyl groups and related branched isomers.

Preferably the alkenyl group is a straight chain or branched alkenyl group with 2 to 8 carbons.

The alkynyl group may be a straight chain or branched alkynyl group having 2-12 carbon atoms. Examples include ethynyl-, 1-propynyl-, 2-propynyl-, 1-butynyl-, 2-butynyl-, 3-butynyl-, 1-pentynyl-, 2-pentynyl-, 3-pentynyl-, 4-pentynyl-, 1-hexynyl-, 2-hexynyl-, 3-hexynyl-, 4-hexynyl-, 5-hexynyl-, 1-heptynyl-, 2-heptynyl-, 3-heptynyl-, 4-heptynyl-, 5-heptynyl-, 6-heptynyl-, 1-octynyl-, 2-octynyl-, 3-octynyl-, 4-octynyl-, 5-octynyl-, 6-octynyl-, 7-octynyl-, 1-nonynyl-, 2-nonynyl-, 3-nonynyl-, 4-nonynyl-, 5-nonynyl-, 6-nonynyl-, 7-nonynyl-, 8-nonynyl-, 1-decynyl-, 2-decynyl-, 3-decynyl-, 4-decynyl-, 5-decynyl-, 6-decynyl-, 7-decynyl-, 8-decynyl-, 9-decynyl-, 1-undecynyl-, 2-undecynyl-, 3-undecynyl-, 4-undecynyl-, 5-undecynyl-, 6-undecynyl-, 7-undecynyl-, 8-undecynyl-, 9-undecynyl-, 10-undecynyl-, 1-dodecynyl-, 2-dodecynyl-, 3-dodecynyl-, 4-dodecynyl-, 5-dodecynyl-, 6-dodecynyl-, 7-dodecynyl-, 8-dodecynyl-, 9-dodecynyl-, 10-dodecynyl- and 11-dodecynyl-groups and related branched isomers.

Preferably the alkynyl group is a straight chain or branched alkenyl group with 2 to 8 carbons.

The cycloalkyl group may include cycloalkyl groups with 3 to 10 carbon atoms. Examples include the cyclopropyl-, cyclobutyl-, cyclopentyl-, cyclohexyl-, cycloheptyl- and cyclooctyl-groups that may be variably substituted at any position(s) around the ring.

Preferably the cycloalkyl group is a cycloalkyl group with 5 to 7 carbon atoms.

The cycloalkenyl group may include cycloalkenyl groups with 3 to 10 carbon atoms. Examples include cyclobutenyl-, cyclopentenyl-, cyclohexenyl-, cycloheptenyl- and cyclooctenyl-groups that may be variably substituted at any position(s) around the ring.

Preferably the cycloalkenyl group is a cycloalkenyl group with 5 to 7 carbon atoms.

The aryl group may include phenyl, biphenyl, naphtyl, anthracenyl groups and the like.

Preferably the aryl group is a phenyl group.

The aryl alkyl group may include a group with 7 to 18 carbons. Examples include benzyl-, 1-methylbenzyl-, 2-phenylethyl-, 3-phenylpropyl-, 4-phenylbutyl-, 5-phenylpentyl-, 6-phenylhexyl-, 1-naphtylmethyl, 2-(1-naphtyl)-ethyl groups and the like.

Preferably the aryl alkyl group is an aryl alkyl group with 7 to 12 carbon atoms.

The heterocyclic group may include 5- to 7-membered heterocyclic groups containing nitrogen, oxygen or sulfur. The heterocyclic ring may be fused with a cyclic or aromatic ring having 3 to 7 carbon atoms such as a benzene, cyclopropyl, cyclobutane, cyclopentane and cyclohexane ring systems. A ring with 5 or 6 carbon atoms is preferred. The heterocyclic ring may be selected from the group consisting of furyl-, dihydrofuranyl-, tetrahydrofuranyl-, dioxolanyl-, oxazolyl-, dihydrooxazolyl-, oxazolidinyl-, isoxazolyl-, dihydroisoxazolyl-, isoxazolidinyl-, oxathiolanyl-, thienyl-, tetrahydrothienyl-, dithiolanyl-, thiazolyl-, dihydrothiazolyl-, thiazolidinyl-, isothiazolyl-, dihydroisothiazolyl-, isothiazolidinyl-, pyrrolyl-, dihydropyrrolyl-, pyrrolidinyl-, pyrazolyl-, dihydropyrazolyl-, pyrazolidinyl-, imidazolyl-, dihydroimidazolyl-, imidazolidinyl-, triazolyl-, dihydrotriazolyl- triazolidinyl-, tetrazolyl-, dihydrotetrazolyl-, tetrazolidinyl-, pyridyl-, dihydropyridyl-, piperidinyl-, morpholinyl-, dioxanyl-, oxathianyl-, trioxanyl-, thiomorpholinyl-, pyridazinyl-, dihydropyridazinyl-, tetrahydropyridazinyl-, hexahydropyridazinyl-, pyrimidinyl-, dihydropyrimadinyl-, tetrahydropyrimadinyl-, hexahydropyrimadinyl-, pyrazinyl-, piperazinyl-, pyranyl-, dihydropyranyl-, tetrahydropyranyl-, thiopyranyl-, dihydrothiopyranyl-, tetrahydrothiopyranyl-, dithianyl-, purinyl-, pyrimidinyl-, pyrrolizinyl-, pyrrolizidinyl, indolyl-, dihydroindolyl-, isoindolyl-, indolizinyl-, indolizidinyl-, quinolyl-, dihydroquinolyl-, tetrahydroquinolyl-, isoquinolyl-, dihydroquinolyl-, tetrahydroquinolyl-, quinolizinyl-, quinolizidinyl-, phenanthrolinyl-, chromenyl-, chromanyl-, isochromenyl-, isochromanyl-, benzofuranyl-, carbazolyl-groups and the like.

The alkylamino group may include a straight chain or branched alkylamino group having 2-12 carbon atoms such as methylamino, ethylamino, propylamino, isopropylamino, butylamino, isobutylamino, tert-butylamino, pentylamino, hexylamino, heptylamino or octylamino.

The alkenyl amino group may include a straight chain or branched alkenylamino group having 2-12 carbon atoms such as ethynylamino-, 1-propynylamino-, 2-propynylamino-, 1-butynylamino-, 2-butynylamino-, 3-butynylamino-, 1-pentynylamino-, 2-pentynylamino-, 3-pentynylamino-, 4-pentynylamino-, 1-hexynylamino-, 2-hexynylamino-, 3-hexynylamino-, 4-hexynylamino-, 5-hexynylamino-, 1-heptynylamino-, 2-heptynylamino-, 3-heptynylamino-, 4-heptynylamino-, 5-heptynylamino-, 6-heptynylamino-, 1-octynylamino-, 2-octynylamino-, 3-octynylamino-, 4-octynylamino-, 5-octynylamino-, 6-octynylamino-, 7-octynylamino-, 1-nonynylamino-, 2-nonynylamino-, 3-nonynyl-amino, 4-nonynylamino-, 5-nonynylamino-, 6-nonynylamino-, 7-nonynylamino-, 8-nonynylamino-, 1-decynylamino-, 2-decynylamino-, 3-decynylamino-, 4-decynylamino-, 5-decynylamino-, 6-decynylamino-, 7-decynylamino-, 8-decynylamino-, 9-decynylamino-, 1-undecynylamino-, 2-undecynylamino-, 3-undecynylamino-, 4-undecynylamino-, 5-undecynylamino-, 6-undecynylamino-, 7-undecynylamino-, 8-undecynylamino-, 9-undecynylamino-, 10-undecynylamino-, 1-dodecynylamino-, 2-dodecynylamino-, 3-dodecynylamino-, 4-dodecynylamino-, 5-dodecynylamino-, 6-dodecynylamino-, 7-dodecynylamino-, 8-dodecynylamino-, 9-dodecynylamino-, 10-dodecynylamino- and 11-dodecynylamino-groups and related branched isomers.

Preferably the alkenyl amino group is a straight chain or branched alkenylamino group with 2 to 8 carbons.

The arylamino group may include arylamino groups such as a phenylamino or naphtylamino group which may be optionally substituted with an alkyl or alkenyl or alkoxy group having 1 to 4 carbon atoms, and also halogens, e.g. phenylamino, 2-methylphenylamino, 3-methylphenylamino, 4-methylphenylamino, 2-allylphenylamino, 2-chlorophenylamino, 3-chlorophenylamini, 4-chlorophenylamino, 4-methoxyphenylamino, 2-allyloxyphenylamino, naphtylamino and the like.

The arylalkylamino group may include benzylamino and 2-phenylethylamino.

The alkylthio group may include alkylthio groups having 1 to 8 carbon atoms such as methylthio, ethylthio, propylthio, butylthio, isobutylthio, pentylthio.

The alkenylthio group may include a straight chain or branched alkenylthio group having 1 to 8 carbon atoms such as ethynylthio-, 1-propynylthio-, 2-propynylthio-, 1-butynylthio-, 2-butynylthio-, 3-butynylthio-, 1-pentynylathio-, 2-pentynylthio-, 3-pentynylthio-, 4-pentynylthio-, 1-hexynylthio-, 2-hexynylthio-, 3-hexynylthio-, 4-hexynylthio-, 5-hexynylthio- and the like.

The arylrthio group may include alkenylthio groups having 1 to 8 carbon atoms such as a phenylthio or naphtylthio group which may be optionally substituted with an alkyl or alkenyl or alkoxy group having 1 to 4 carbon atoms, and also halogens, e.g. phenylthio, 2-methylphenylthio, 3-methylphenylthio, 4-methylphenylthio, 2-allylphenylthio, 2-chlorophenylthio, 3-chlorophenylamini, 4-chlorophenylthio, 4-methoxyphenylthio, 2-allyloxyphenylthio, naphtylthio and the like.

The arylalkylthio group may include alkenylthio groups having 1 to 8 carbon atoms such as the benzylthio-group and 2-phenylethylthio-group.

The alkoxycarbonyl group may include methoxycarbonyl, ethoxycarbonyl and the like,

The substituted or unsubstituted carbamoyl group may include carbamoyl, methylcarbamoyl, dimethylcarbamoyl, diethylcarbamoyl and the like.

The acyl group may include acyl groups with 1 to 8 carbon atoms such as formyl, acetyl, propionyl or benzoyl groups and others.

The alkyl, alkenyl, alkynyl, cycloalkyl, aryl, aryl alkyl, heterocyclic, alkylamino, alkenylamino, arylamino, arylalkylamino, alkylthio, alkenylthio, arylthio, arylalkylthio, alkoxycarbonyl, substituted and unsubstituted carbamoyl and acyl groups mentioned above may optionally be substituted. Examples of such substituents include halogens (F, Cl, Br, I), hydroxyl groups, mercapto groups, carboxylates, nitro groups, cyano groups, substituted or unsubstituted amino groups (including amino, methylamino, dimethylamino, ethylamino, diethylamino, and various protected amines such as tert-butoxycarbonyl- and arylsulfonamido groups), alkoxy groups (having 1 to 8 carbon atoms such as methoxy, ethoxy, propyloxy, isopropyloxy, butyloxy, isobutyloxy, tert-butyloxy, pentyloxy, hexyloxy, heptyloxy or octyloxy), alkenyloxy groups (having 2 to 8 carbon atoms such as a vinyloxy, allyloxy, 3-butenyloxy or 5-hexenyloxy), aryloxy groups (such as a phenoxy or naphtyloxy group which may be optionally substituted with an alkyl or alkenyl or alkoxy group having 1 to 4 carbon atoms, and also halogens, e.g. phenoxy, 2-methylphenoxy, 3-methylphenoxy, 4-methylphenoxy, 2-allylphenoxy, 2-chlorophenoxy, 3-chlorophenoxy, 4-chlorophenoxy, 4-methoxyphenoxy, 2-allyloxyphenoxy, naphtyloxy and the like), aryl alkyloxy groups (e.g. benzyloxy and 2-phenylethyloxy), alkylthio groups (having 1 to 8 carbon atoms such as methylthio, ethylthio, propylthio, butylthio, isobutylthio, pentylthio), alkoxycarbonyl groups (e.g. methoxycarbonyl, ethoxycarbonyl and the like), substituted or unsubstituted carbamoyl group (e.g. carbamoyl, methylcarbamoyl, dimethylcarbamoyl, diethylcarbamoyl and the like), acyl groups (with 1 to 8 carbon atoms such as formyl, acetyl, propionyl or benzoyl groups) and others.

The above-mentioned cycloalkyl, cycloalkenyl, aryl, aryl alkyl, heterocyclic, alkoxy, alkenyloxy, aryloxy, aryl alkyloxy, alkylthio, and alkoxycarbonyl groups may also be substituted with alkyl groups having 1 to 5 carbon atoms, alkenyl groups with 2 to 5 carbon atoms, or haloalkyl groups with 1 to 5 carbon atoms in addition to the substituents specified above.

The number of substituents may be one or more than one.

The substituents may be the same or different.

R can also take the form of R′—X, where X is a functional group bonded to any carbon of R′ except C₁. The functional group may be for example a halogen (F, Cl, Br, I), hydroxyl group, mercapto group, carboxylate, nitro group, cyano group, substituted or unsubstituted amino group (including amino, methylamino, dimethylamino, ethylamino, diethylamino), and various protected amines such as a tert-butoxycarbonyl- or a arylsulfonamido group.

—OR as a whole can also be replaced by a functional group such as a halogen (F, Cl, Br, I), hydroxyl group, mercapto group, carboxylate, nitro group, cyano group, substituted or unsubstituted amino group (including amino, methylamino, dimethylamino, ethylamino, diethylamino), and various protected amines such as a tert-butoxycarbonyl- or a arylsulfonamido group.

“Polypeptide” and “protein” are used interchangeably and mean any peptide-linked chain of amino acids, regardless of length or post-translational modification. The invention also features yeast enantioselective glycidyl ether hydrolase (YEGH) polypeptides with conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

The term “isolated” polypeptide or peptide fragment, as used herein, refers to a polypeptide or a peptide fragment which either has no naturally-occurring counterpart or has been separated or purified from components which naturally accompany it, e.g., microorganism cellular components such as yeast cell cellular components. Typically, the polypeptide or peptide fragment is considered “isolated” when it is at least 70%, by dry weight, free from the proteins and other naturally-occurring organic molecules with which it is naturally associated. Preferably, a preparation of a polypeptide (or peptide fragment thereof) of the invention is at least 80%, more preferably at least 90%, and most preferably at least 99%, by dry weight, the polypeptide (or the peptide fragment thereof), respectively, of the invention. Thus, for example, a preparation of polypeptide x is at least 80%, more preferably at least 90%, and most preferably at least 99%, by dry weight, polypeptide x. Since a polypeptide that is chemically synthesized is, by its nature, separated from the components that naturally accompany it, the synthetic polypeptide is “isolated.”

An isolated polypeptide (or peptide fragment) of the invention can be obtained, for example, by: extraction from a natural source (e.g., from yeast cells); expression of a recombinant nucleic acid encoding the polypeptide; or chemical synthesis. A polypeptide that is produced in a cellular system different from the source from which it naturally originates is “isolated,” because it will necessarily be free of components which naturally accompany it. The degree of isolation or purity can be measured by any appropriate method, e.g., column chromatography, polyacrylamide gel electrophoresis, or HPLC analysis.

An “isolated DNA” is either (1) a DNA that contains sequence not identical to that of any naturally occurring sequence, or (2), in the context of a DNA with a naturally-occurring sequence (e.g., a cDNA or genomic DNA), a DNA free of at least one of the genes that flank the gene containing the DNA of interest in the genome of the organism in which the gene containing the DNA of interest naturally occurs. The term therefore includes a recombinant DNA incorporated into a vector, into an autonomously replicating plasmid or virus, or into the genomic DNA of a prokaryote or eukaryote. The term also includes a separate molecule such as: a cDNA (e.g., SEQ ID NOs: 10, 11, 12, 13, 14, 15, 16, 17, or 18) where the corresponding genomic DNA can include introns and therefore can have a different sequence; a genomic fragment that lacks at least one of the flanking genes; a fragment of cDNA or genomic DNA produced by polymerase chain reaction (PCR) and that lacks at least one of the flanking genes; a restriction fragment that lacks at least one of the flanking genes; a DNA encoding a non-naturally occurring protein such as a fusion protein, mutein, or fragment of a given protein; and a nucleic acid which is a degenerate variant of a cDNA or a naturally occurring nucleic acid. In addition, it includes a recombinant nucleotide sequence that is part of a hybrid gene, i.e., a gene encoding a non-naturally occurring fusion protein. Also included is a recombinant DNA that includes a portion of SEQ ID NOs: 10, 11, 12, 13, 14, 15, 16, 17, or 18. It will be apparent from the foregoing that isolated DNA does not mean a DNA present among hundreds to millions of other DNA molecules within, for example, cDNA or genomic DNA libraries or genomic DNA restriction digests in, for example, a restriction digest reaction mixture or an electrophoretic gel slice.

As used herein, a “functional fragment” of a YEGH polypeptide is a fragment of the polypeptide that is shorter than the full-length polypeptide and has at least 20% (e.g., at least: 30%; 40%; 50%; 60%; 70%; 80%; 90%; 95%; 98%; 99%; 100%, or more) of the ability of the full-length polypeptide to enantioselectively hydrolyse a GE of interest. Fragments of interest can be made by either recombinant, synthetic, or proteolytic digestive methods and tested for their ability to enantioselectively hydrolyse a GE.

As used herein, “operably linked” means incorporated into a genetic construct so that an expression control sequence effectively controls expression of a coding sequence of interest.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., glycidyl ether (GE) and associated vicinol diol (GD) substantially enriched for one optical enantiomer, will be apparent from the following description, from the drawings and from the claims.

### DESCRIPTION OF DRAWINGS

FIG. 1 shows vector pYLHmA. Restriction enzyme sites indicate unique sites available for insertion of genes under control of the hp4d promoter and LIP2 terminator.

FIG. 2 shows vector pYLTsA. Restriction enzyme sites indicate the unique sites available for insertion of genes under control of the TEF promoter and LIP2 terminator.

FIGS. 3A-3M (examples 56-68) show hydrolysis of (±)-phenyl glycidyl ether by selected wild type yeasts to produce optically active (R)-phenyl glycidyl ether and the corresponding (S)-diol.

FIGS. 4A-4G (examples 69-75) show hydrolysis of (±)-phenyl ether by recombinant yeast expression hosts transformed with the epoxide hydrolase genes from selected wild type yeast strains to produce optically active (R)-phenyl glycidyl ether and the corresponding (S)-diol.

FIGS. 5A-5D (examples 177-180) show hydrolysis of (±)-benzyl glycidyl ether by selected wild type yeasts to produce optically active (S)-benzyl glycidyl ether and the corresponding (S)-diol.

FIGS. 6A and 6B (examples 181 and 182) shows hydrolysis of (±)-benzyl glycidyl ether by selected wild type yeast to produce optically active (R)-benzyl glycidyl ether and the corresponding (R)-diol.

FIGS. 7A-7E (examples 183-187) show hydrolysis of (±)-benzyl glycidyl ether by recombinant yeast expression hosts transformed with the epoxide hydrolase genes from selected wild type yeast strains to produce optically active (R)-benzyl glycidyl ether and the corresponding (R)-3-benzyloxy-1,2-propanediol.

FIGS. 8A-8E (examples 255-259) shows hydrolysis of (±)-furfuryl glycidyl ether by selected wild type yeasts to produce optically active (R)-furfuryl glycidyl ether and the corresponding (R)-diol.

FIGS. 9A-9D (examples 260-263) shows hydrolysis of (±)-furfuryl glycidyl ether by recombinant yeast expression hosts transformed with the epoxide hydrolase genes from selected wild type yeast strains to produce optically active (R)-furfuryl glycidyl ether and the corresponding (R)-furfuryloxy-1,2-propanediol.

FIGS. 10A-10B (examples 296-297) shows hydrolysis of (±)-isopropyl glycidyl ether by selected wild type yeasts to produce optically active (R)-isopropyl glycidyl ether and the corresponding enriched (S)-diol.

FIGS. 11A and 11B (examples 298 and 299) shows hydrolysis of (±)-isopropyl glycidyl ether by recombinant yeast expression hosts transformed with the epoxide hydrolase genes from selected wild type yeast strains to produce optically active (R)-isopropyl glycidyl ether and the corresponding (S)-3-isopropyloxy-1,2-propanediol.

FIGS. 12A and 12B (examples 300 and 301) shows hydrolysis of (±)-glycidyl tosylate by recombinant yeast expression hosts transformed with the epoxide hydrolase genes from selected wild type yeast strains to produce optically active (R)-glycidyl tosylate and the corresponding (S)-diol.

FIG. 13A to 13D (examples 302 and 305) shows hydrolysis of (±) 1-(naphth-2-yloxy)-2,3-epoxypropane by recombinant yeast expression hosts transformed with the epoxide hydrolase genes from selected wild type yeast strains to produce optically active (R)-1-(naphth-2-yloxy)-2,3-epoxypropane and the corresponding (S) diol.

FIGS. 14 to 22 are the amino acid sequences for yeast epoxide hydrolases (allocated amino acid SEQ. ID. NOS. 1 to 9 respectively) derived from various yeast strains for the production of optically active glycidyl ethers and diols from racemic glycidyl ethers

FIGS. 23 to 31 are the nucleotide sequences for yeast epoxide hydrolases (allocated nucleotide SEQ. ID. NOS. 10 to 18 respectively) derived from various yeast strains for the production of optically active glycidyl ethers and diols from racemic glycidyl ethers

FIG. 32 is a table showing the homology at the amino acid level of yeast epoxide hydrolases that are enantioselective on hydrolysis of glycidyl ethers.

FIG. 33 is a table showing the homology at the nucleotide level of yeast epoxide hydrolases that are enantioselective on hydrolysis of glycidyl ethers.

FIG. 34 shows the amino acid alignments of yeast epoxide hydrolase proteins, indicating conserved sequence motifs and regions surrounding the catalytic triad.

### DETAILED DESCRIPTION

Various aspects of the invention are described below.

### Nucleic Acid Molecules

The YEGH nucleic acid molecules of the invention can be cDNA, genomic DNA, synthetic DNA, or RNA, and can be double-stranded or single-stranded (i.e., either a sense or an antisense strand). Segments of these molecules are also considered within the scope of the invention, and can be produced by, for example, the polymerase chain reaction (PCR) or generated by treatment with one or more restriction endonucleases. A ribonucleic acid (RNA) molecule can be produced by in vitro transcription. Preferably, the nucleic acid molecules encode polypeptides that, regardless of length, are soluble under normal physiological conditions.

The nucleic acid molecules of the invention can contain naturally occurring sequences, or sequences that differ from those that occur naturally, but, due to the degeneracy of the genetic code, encode the same polypeptide (for example, one of the polypeptides with SEQ ID NOS: 1-9). In addition, these nucleic acid molecules are not limited to coding sequences, e.g., they can include some or all of the non-coding sequences that lie upstream or downstream from a coding sequence.

The nucleic acid molecules of the invention can be synthesized (for example, by phosphoramidite-based synthesis) or obtained from a biological cell, such as the cell of a eukaryote (e.g., a mammal such as human or a mouse or a yeast such as any of the genera, species, and strains of yeast disclosed herein) or a prokaryote (e.g., a bacterium such as Escherichia coli). The nucleic acids can be those of a yeast such as any of the genera, species, and strains of yeast disclosed herein. Combinations or modifications of the nucleotides within these types of nucleic acids are also encompassed.

In addition, the isolated nucleic acid molecules of the invention encompass segments that are not found as such in the natural state. Thus, the invention encompasses recombinant nucleic acid molecules (for example, isolated nucleic acid molecules encoding the polypeptides of SEQ. ID. NOs: 1-9) incorporated into a vector (for example, a plasmid or viral vector) or into the genome of a heterologous cell (or the genome of a homologous cell, at a position other than the natural chromosomal location). Recombinant nucleic acid molecules and uses therefor are discussed further below.

Techniques associated with detection or regulation of genes are well known to skilled artisans. Such techniques can be used, for example, to test for expression of a YEGH gene in a test cell (e.g., a yeast cell) of interest.

A YEGH family gene or protein can be identified based on its similarity to the relevant YEGH gene or protein, respectively. For example, the identification can be based on sequence identity. The invention features isolated nucleic acid molecules which are, or are at least 50% (e.g., at least: 55%; 60%; 65%; 75%; 85%; 95%; 98%; or 99%) identical to: (a) a nucleic acid molecule that encodes the polypeptide of SEQ ID NOs: 1-9; (b) the nucleotide sequence of SEQ ID NOs:10-18; (c) a nucleic acid molecule which includes a segment of at least 15 (e.g., at least: 20; 25; 30; 35; 40; 50; 60; 80; 100; 125; 150; 175; 200; 250; 300; 350; 400; 500; 600; 700; 800; 900; 1,000; 1,100; 1,150; 1,160; 1,170; 1,175; 1,178; 1,180; 1,181; 1,200; 1,220; 1,225; 1,226; 1,228; 1,230; 1,231; or 1,232) nucleotides of SEQ ID NOs:10-18; (d) a nucleic acid molecule encoding any of the polypeptides or fragments thereof disclosed below; and (e) the complement of any of the above nucleic acid molecules. The complements of the above molecules can be full-length complements or segment complements containing a segment of at least 15 (e.g., at least: 20; 25; 30; 35; 40; 50; 60; 80; 100; 125; 150; 175; 200; 250; 300; 350; 400; 500; 600; 700; 800; 900; 1,000; 1,100; 1,200; 1,220; 1,225; 1,228; 1,230; 1,231; or 1,232) consecutive nucleotides complementary to any of the above nucleic acid molecules. Identity can be over the full-length of SEQ ID NOs: 10-18 or over one or more contiguous or non-contiguous segments.

The determination of percent identity between two sequences is accomplished using the mathematical algorithm of Karlin and Altschul, Proc. Natl. Acad. Sci. USA 90, 5873-5877, 1993. Such an algorithm is incorporated into the BLASTN and BLASTP programs of Altschul et al. (1990) J. Mol. Biol. 215, 403-410. BLAST nucleotide searches are performed with the BLASTN program, score=100, wordlength=12, to obtain nucleotide sequences homologous to HIN-1-encoding nucleic acids. BLAST protein searches are performed with the BLASTP program, score=50, wordlength=3, to obtain amino acid sequences homologous to the HIN-1 polypeptide. To obtain gap alignments for comparative purposes, Gap BLAST is utilized as described in Altschul et al. (1997) Nucleic Acids Res. 25, 3389-3402. When utilizing BLAST and Gap BLAST programs, the default parameters of the respective programs (e.g., XBLAST and NBLAST) are used.

Hybridization can also be used as a measure of homology between two nucleic acid sequences. A YEGH-encoding nucleic acid sequence, or a portion thereof, can be used as a hybridization probe according to standard hybridization techniques. The hybridization of a YEGH probe to DNA or RNA from a test source (e.g., a mammalian cell) is an indication of the presence of YEGH DNA or RNA in the test source. Hybridization conditions are known to those skilled in the art and can be found in Current Protocols in Molecular Biology, John Wiley & Sons, N.Y., 6.3.1-6.3.6, 1991. Moderate hybridization conditions are defined as equivalent to hybridization in 2× sodium chloride/sodium citrate (SSC) at 30° C., followed by a wash in 1×SSC, 0.1% SDS at 50° C. Highly stringent conditions are defined as equivalent to hybridization in 6× sodium chloride/sodium citrate (SSC) at 45° C., followed by a wash in 0.2×SSC, 0.1% SDS at 65° C.

The invention also encompasses: (a) vectors (see below) that contain any of the foregoing YEGH coding sequences (including coding sequence segments) and/or their complements (that is, “antisense” sequences); (b) expression vectors that contain any of the foregoing YEGH coding sequences (including coding sequence segments) operably linked to one or more transcriptional and/or translational regulatory elements (TRE; examples of which are given below) necessary to direct expression of the coding sequences; (c) expression vectors encoding, in addition to a YEGH polypeptide (or a fragment thereof), a sequence unrelated to YEGH, such as a reporter, a marker, or a signal peptide fused to YEGH; and (d) genetically engineered host cells (see below) that contain any of the foregoing expression vectors and thereby express the nucleic acid molecules of the invention.

Recombinant nucleic acid molecules can contain a sequence encoding a YEGH polypeptide or a YEGH polypeptide having an heterologous signal sequence. The full length YEGH polypeptide, or a fragment thereof, can be fused to such heterologous signal sequences or to additional polypeptides, as described below. Similarly, the nucleic acid molecules of the invention can encode a YEGH that includes an exogenous polypeptide that facilitates secretion.

The TRE referred to above and further described below include but are not limited to inducible and non-inducible promoters, enhancers, operators and other elements that are known to those skilled in the art and that drive or otherwise regulate gene expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast-mating factors. Other useful TRE are listed in the examples below.

Similarly, the nucleic acid can form part of a hybrid gene encoding additional polypeptide sequences, for example, a sequence that functions as a marker or reporter. Examples of marker and reporter genes include -lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacZ (encoding -galactosidase), xanthine guanine phosphoribosyltransferase (XGPRT), and green, yellow, or blue fluorescent protein. As with many of the standard procedures associated with the practice of the invention, skilled artisans will be aware of additional useful reagents, for example, additional sequences that can serve the function of a marker or reporter. Generally, the hybrid polypeptide will include a first portion and a second portion; the first portion being a YEGH polypeptide (or any of YEGH fragments described below) and the second portion being, for example, the reporter described above or an Ig heavy chain constant region or part of an Ig heavy chain constant region, e.g., the CH2 and CH3 domains of IgG2a heavy chain. Other hybrids could include an antigenic tag or a poly-His tag to facilitate purification.

The expression systems that can be used for purposes of the invention include, but are not limited to, microorganisms such as yeasts (e.g., any of the genera, species or strains listed herein) or bacteria (for example, E. coli and B. subtilis) transformed with recombinant bacteriophage DNA, plasmid DNA, or cosmid DNA expression vectors containing the nucleic acid molecules of the invention; yeast (for example, Saccharomyces, Kluyveromyces, Hansenula, Pichia, Yarrowia, Arxula and Candida, and other genera, species, and strains listed herein) transformed with recombinant yeast expression vectors containing the nucleic acid molecule of the invention; insect cell systems infected with recombinant virus expression vectors (for example, baculovirus) containing the nucleic acid molecule of the invention; plant cell systems infected with recombinant virus expression vectors (for example, cauliflower mosaic virus (CaMV) or tobacco mosaic virus (TMV)) or transformed with recombinant plasmid expression vectors (for example, Ti plasmid) containing a YEGH nucleotide sequence; or mammalian cell systems (for example, COS, CHO, BHK, 293, VERO, HeLa, MDCK, W138, and NIH 3T3 cells) harboring recombinant expression constructs containing promoters derived from the genome of mammalian cells (for example, the metallothionein promoter) or from mammalian viruses (for example, the adenovirus late promoter and the vaccinia virus 7.5K promoter). Also useful as host cells are primary or secondary cells obtained directly from a mammal and transfected with a plasmid vector or infected with a viral vector.

The invention includes wild-type and recombinant cells including, but not limited to, yeast cells (e.g., any of those disclosed herein) containing any of the above YEGH genes, nucleic acid molecules, and genetic constructs. Other cells that can be used as host cells are listed herein. The cells are preferably isolated cells. As used herein, the term “isolated” as applied to a microorganism (e.g., a yeast cell) refers to a microorganism which either has no naturally-occurring counterpart (e.g., a recombinant microorganism such as a recombinant yeast) or has been extracted and/or purified from an environment in which it naturally occurs. Thus, an “isolated microorganism” does not include one residing in an environment in which it naturally occurs, for example, in the air, outer space, the ground, oceans, lakes, rivers, and streams and the like, ground at the bottom of oceans, lakes, rivers, and streams and the like, snow, ice on top of the ground or in/on oceans lakes, rivers, and streams and the like, man-made structures (e.g., buildings), or in natural hosts (e.g., plant, animal or microbial hosts) of the microorganism, unless the microorganism (or a progenitor of the microorganism) was previously extracted and/or purified from an environment in which it naturally occurs and subsequently returned to such an environment or any other environment in which it can survive. An example of an isolated microorganism is one in a substantially pure culture of the microorganism.

Moreover the invention provides a substantially pure culture of a microorganism (e.g., a microbial cell such as a yeast cell). As used herein, a “substantially pure culture” of a microorganism is a culture of that microorganism in which less than about 40% (i.e., less than about: 35%; 30%; 25%; 20%; 15%; 10%; 5%; 2%; 1%; 0.5%; 0.25%; 0.1%; 0.01%; 0.001%; 0.0001%; or even less) of the total number of viable microbial (e.g., bacterial, fungal (including yeast), mycoplasmal, or protozoan) cells in the culture are viable microbial cells other than the microorganism. The term “about” in this context means that the relevant percentage can be 15% percent of the specified percentage above or below the specified percentage. Thus, for example, about 20% can be 17% to 23%. Such a culture of microorganisms includes the microorganisms and a growth, storage, or transport medium. Media can be liquid, semi-solid (e.g., gelatinous media), or frozen. The culture includes the cells growing in the liquid or in/on the semi-solid medium or being stored or transported in a storage or transport medium, including a frozen storage or transport medium. The cultures are in a culture vessel or storage vessel or substrate (e.g., a culture dish, flask, or tube or a storage vial or tube).

The microbial cells of the invention can be stored, for example, as frozen cell suspensions, e.g., in buffer containing a cryoprotectant such as glycerol or sucrose, as lyophilized cells. Alternatively, they can be stored, for example, as dried cell preparations obtained, e.g., by fluidised bed drying or spray drying, or any other suitable drying method. Similarly the enzyme preparations can be frozen, lyophilised, or immobilized and stored under appropriate conditions to retain activity.

### Polypeptides and Polypeptide Fragments

The YEGH polypeptides of the invention include all the YEGH and fragments of YEGH disclosed herein. They can be, for example, the polypeptides with SEQ ID NOs: 1-9 and functional fragments of these polypeptides. The polypeptides embraced by the invention also include fusion proteins that contain either full-length or a functional fragment of it fused to unrelated amino acid sequence. The unrelated sequences can be additional functional domains or signal peptides.

The invention features isolated polypeptides which are, or are at least 50% (e.g., at least: 55%; 60%; 65%; 75%; 85%; 95%; 98%; or 99%) identical to the polypeptides with SEQ ID NOs: 1-9. The identity can be over the full-length of the latter polypeptides or over one or more contiguous or non-contiguous segments.

Fragments of YEGH polypeptide are segments of the full-length YEGH polypeptide that are shorter than full-length YEGH. Fragments of YEGH can contain 5-410 (e.g., 10, 20, 30, 40, 50, 60, 70, 80, 90, 100, 120, 150, 250, 300, 350, 380, 390, 391, 392, 393, 400, 405, 406, 407, 408, 409, or 410) amino acids of SEQ ID NOs: 1-9. Fragments of YEGH can be functional fragments or antigenic fragments.

The polypeptides can be any of those described above but with not more 50 (e.g., not more than 50, 45, 40, 35, 30, 25, 20, 17, 14, 12, 10, nine, eight, seven, six, five, four, three, two, or one) conservative substitution(s). Such substitutions can be made by, for example, site-directed mutagenesis or random mutagenesis of appropriate YEGH coding sequences

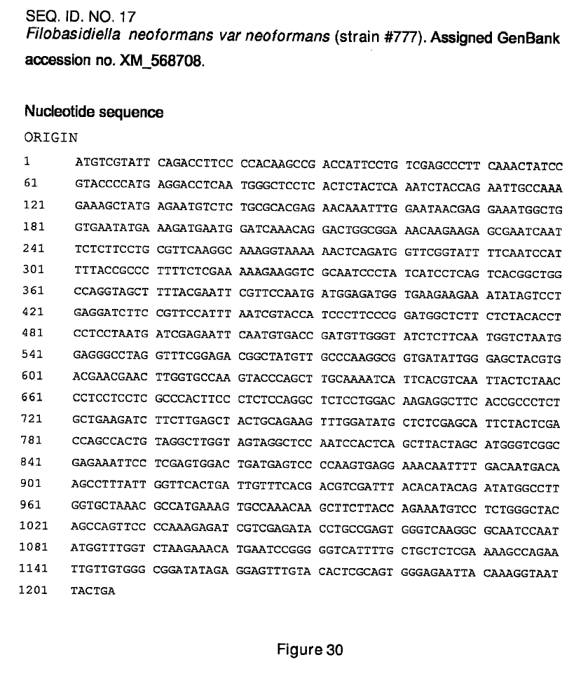

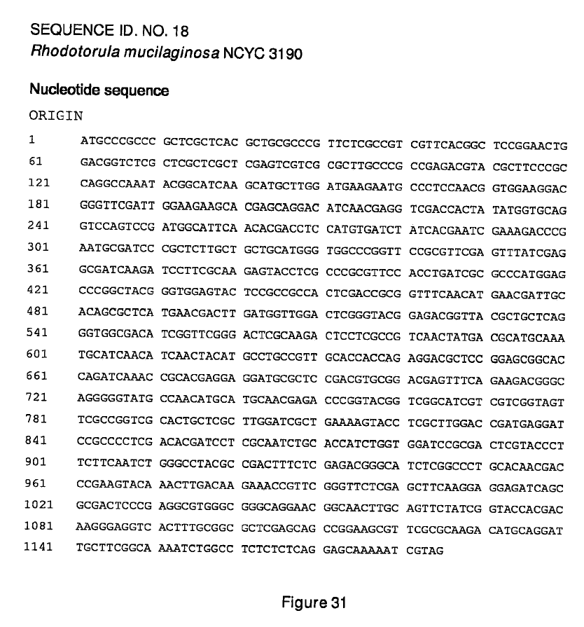

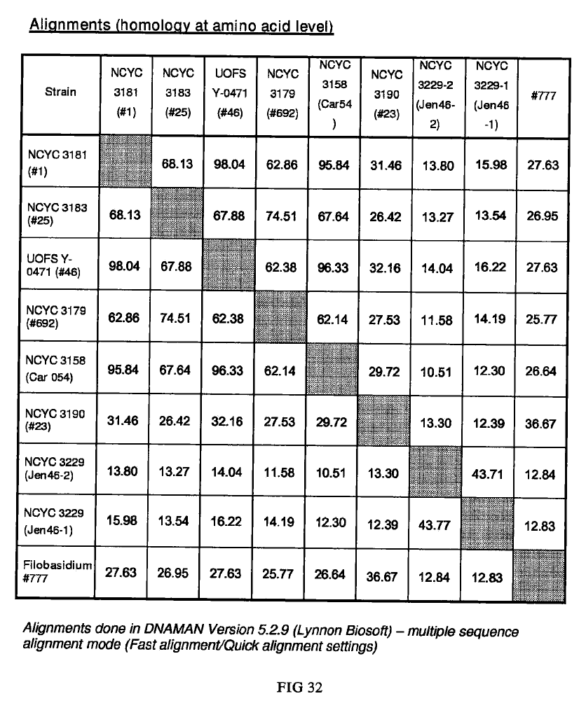

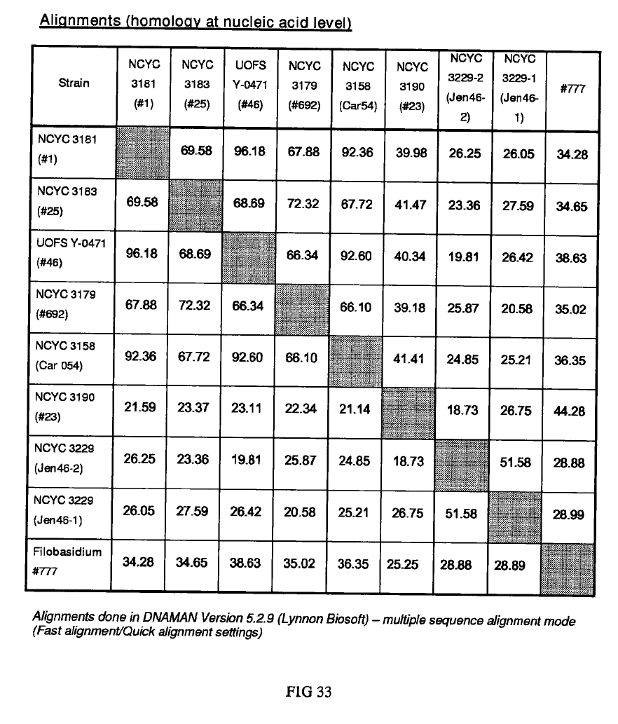

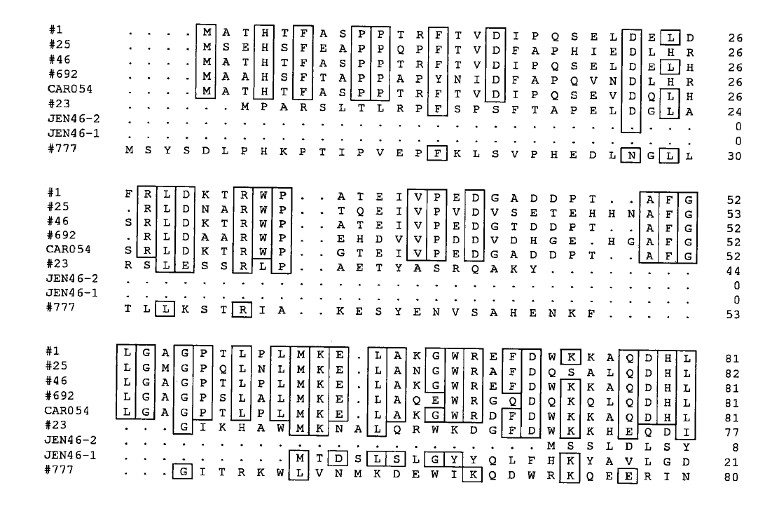

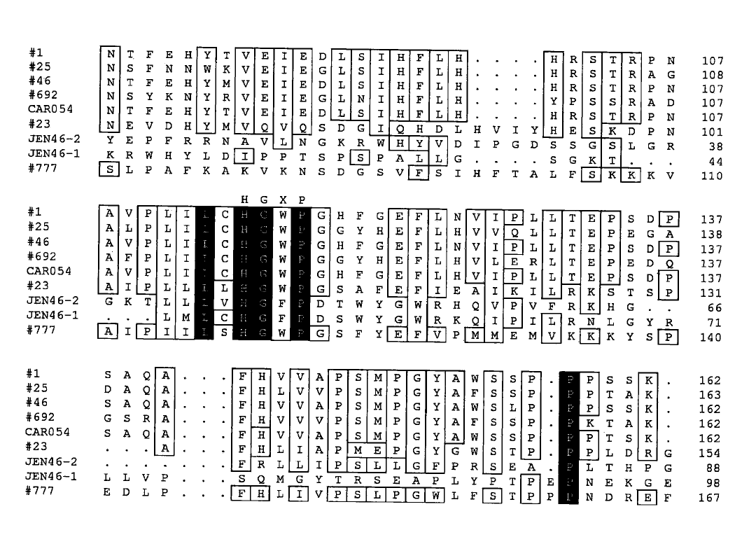

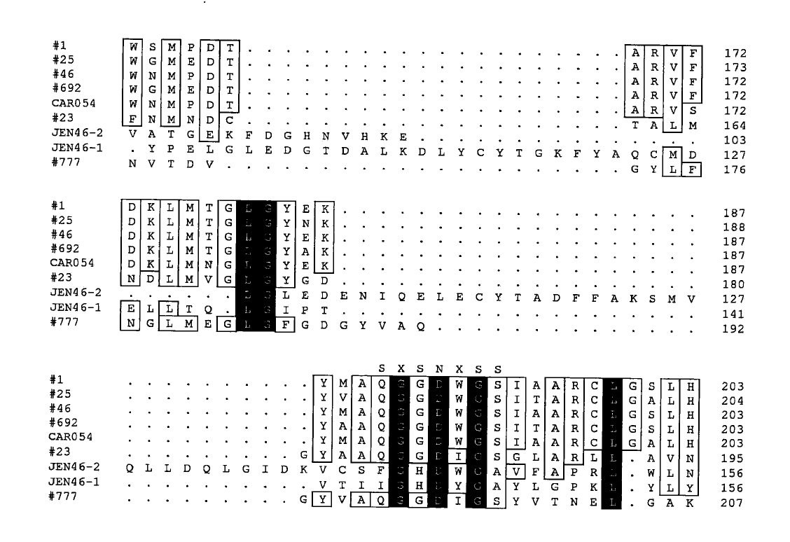

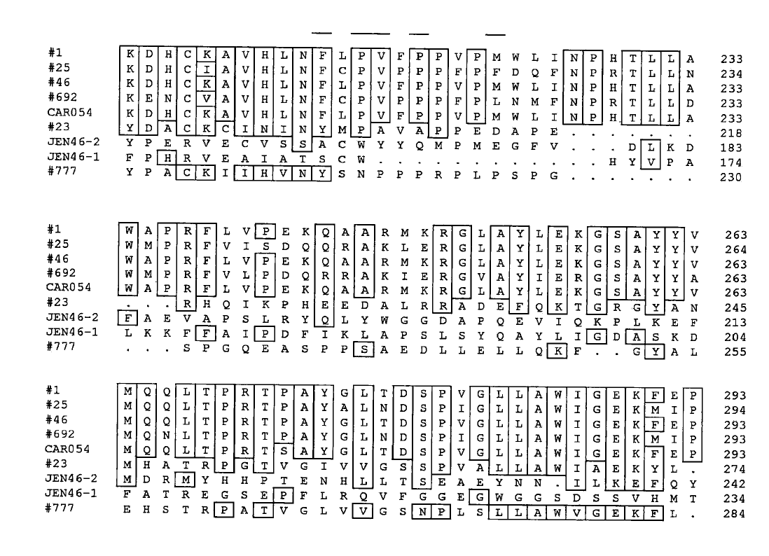

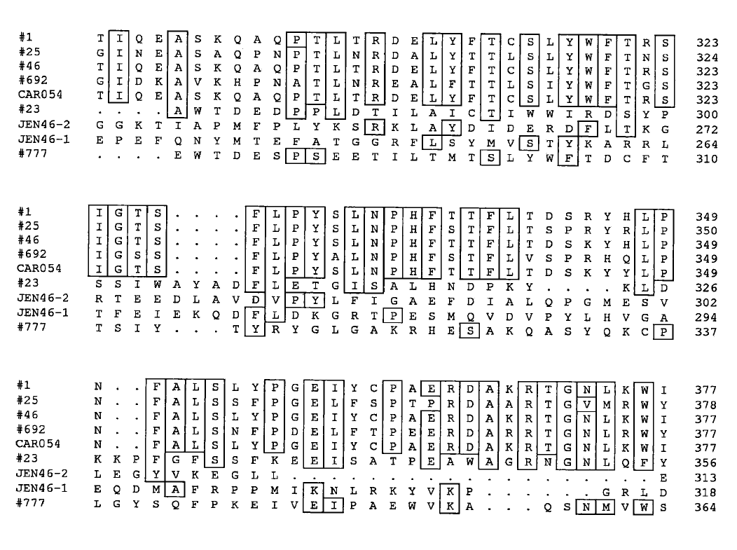

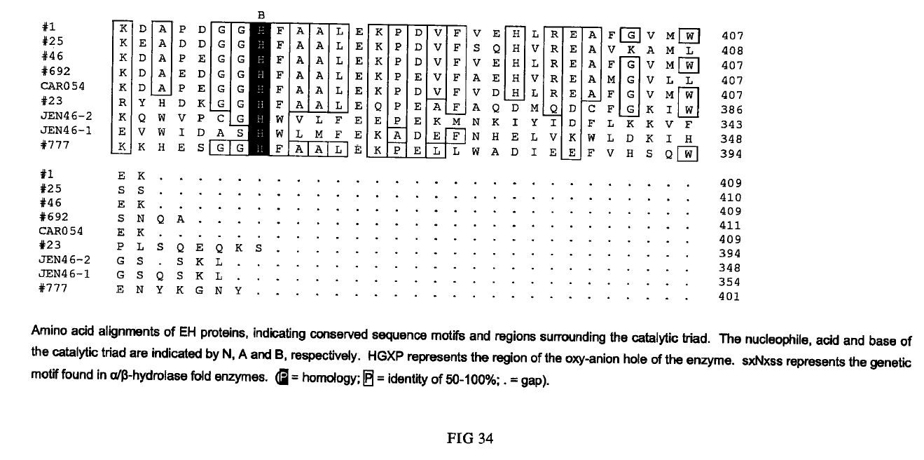

“Functional fragments” of a YEGH polypeptide (and, optionally, any of the above-described YEGH polypeptide variants) have at least 20% (e.g., 30%, 40%, 50%, 60%, 70%, 80%, 90%, 95%, 98%, 99%, 100%, or more) of the ability of the full-length, wild-type YEGH polypeptide to enantioselectively hydrolyse a GE of interest. One of skill in the art will be able to predict YEGH functional fragments using his or her own knowledge and information provided herein, e.g., the amino acid alignments in FIG. 30 showing highly conserved domains and residues required for epoxide hydrolase activity.

Fragments of interest can be made either by recombinant, synthetic, or proteolytic digestive methods and tested for their ability to enantioselectively hydrolyse enantiomers of racemic GE.

Antigenic fragments of the polypeptides of the invention are fragments that can bind to an antibody. Methods of testing whether a fragment of interest can bind to an antibody are known in the art.

The polypeptides can be purified from natural sources (e.g., wild-type or recombinant yeast cells such as any of those described herein). Smaller peptides (e.g., those less than about 100 amino acids in length) can also be conveniently synthesized by standard chemical means. In addition, both polypeptides and peptides can be produced by standard in vitro recombinant DNA techniques and in vivo transgenesis, using nucleotide sequences encoding the appropriate polypeptides or peptides. Methods well-known to those skilled in the art can be used to construct expression vectors containing relevant coding sequences and appropriate transcriptional/translational control signals. See, for example, the techniques described in Sambrook et al., Molecular Cloning: A Laboratory Manual (2nd Ed.) [Cold Spring Harbor Laboratory, N.Y., 1989], and Ausubel et al., Current Protocols in Molecular Biology [Green Publishing Associates and Wiley Interscience, N.Y., 1989].

Polypeptides and fragments of the invention also include those described above, but modified by the addition, at the amino- and/or carboxyl-terminal ends, of a blocking agent to facilitate survival of the relevant polypeptide. This can be useful in those situations in which the peptide termini tend to be degraded by proteases. Such blocking agents can include, without limitation, additional related or unrelated peptide sequences that can be attached to the amino and/or carboxyl terminal residues of the peptide to be administered. This can be done either chemically during the synthesis of the peptide or by recombinant DNA technology by methods familiar to artisans of average skill.

Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the peptides can be covalently or non-covalently coupled to pharmaceutically acceptable “carrier” proteins prior to administration.

Also of interest are peptidomimetic compounds that are designed based upon the amino acid sequences of the functional peptide fragments. Peptidomimetic compounds are synthetic compounds having a three-dimensional conformation (i.e., a “peptide motif”) that is substantially the same as the three-dimensional conformation of a selected peptide. The peptide motif provides the peptidomimetic compound with the ability to enantioselectively hydrolyse a GE of interest in a manner qualitatively identical to that of the YEGH functional fragment from which the peptidomimetic was derived. Peptidomimetic compounds can have additional characteristics that enhance their therapeutic utility, such as increased cell permeability and prolonged biological half-life.

The peptidomimetics typically have a backbone that is partially or completely non-peptide, but with side groups that are identical to the side groups of the amino acid residues that occur in the peptide on which the peptidomimetic is based. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics.

The invention also provides compositions and preparations containing one or more (e.g., two, three, four, five, six, seven, eight, nine, ten, 12, 15, 20, 25, or more) of the above-described polypeptides, polypeptide variants, and polypeptide fragments. The composition or preparation can be, for example a crude cell (e.g., yeast cell) extract or culture supernatant, a crude enzyme preparation, a highly purified enzyme preparation. The compositions and preparations can also contain one or more of a variety of carriers or stabilizers known in the art. Carriers and stabilizers are known in the art and include, for example: buffers, such as phosphate, citrate, and other non-organic acids; antioxidants such as ascorbic acid; low molecular weight (less than 10 residues) polypeptides; proteins such as serum albumin, gelatin or immunoglobulins; hydrophilic polymers such polyvinylpyrrolidone; amino acids such as glycine, glutamine, asparagine, arginine, or lysine; monosaccharides, disaccharides, and other carbohydrates including glucose, mannose, or dextrans; chelating agents such as ethylenediaminetetraacetic acid (EDTA); sugar alcohols such as mannitol, or sorbitol; salt-forming counterions such as sodium; and/or nonionic surfactants such as Tween and Pluronics.

### Methods of Producing Optically Active Glycidyl Ethers and Optically Active Vicinal Diols

The invention provides methods for obtaining enantiopure, or substantially enantiopure, optically active GE and optically active GD. Enantiopure optically active GE or GD preparations are preparations containing one enantiomer of the GE or GD and none of the other enantiomer of the GE or GD. “Substantially enantiopure” optically active GE or GD preparations are preparations containing at least 55% (e.g., at least: 60%; 70%; 80%; 85%; 90%; 95%; 97%; 98%; 99%; 99.5%; 99.8%; or 99.9%), relative to the total amount of both GE or GD enantiomers, of the particular enantiomer of the GE or the GD.

The method involves exposing a GE sample containing a mixture of both enantiomers of the GE to a YEGH polypeptide (e.g., an isolated YEGH polypeptide or one in a microbial cell), which selectively catalyzes the conversion of one of the enantiomers of the GE to a corresponding GD. In this way the desired GD is produced, the selective GE enantiomer substrate for the YEGH is selectively depleted, and the relative proportion (of the total amount of the GE) of the other GE enantiomer is increased. YEGH polypeptides useful for the invention (i.e., those with GE enantioselective activity) will catalyze the conversion of one enantiomer of a GE to its corresponding GD with less than 80% (e.g., less than: 70%, 60%, 50%, 40%, 30%; 20%; 10%; 5%; 2.5%; 1%; 0.5%; 0.01%) of the efficiency that its catalyzes the conversion of the other enantiomer of the GE to its corresponding GD. The starting enantiomeric mixtures can be racemic with respect to the two GE enantiomers or they can contain various proportions of the two GE enantiomers ((e.g., 95:5, 90:10, 80:20, 70:30, 60:40 or 50:50) In addition, optimal concentrations of the GE and conditions of incubation will vary from one YEGH polypeptide to another and from one GE to another. Given the teachings of the working examples contained herein, one skilled in the art will know how to select working conditions for the production of a desired enantiomer of a desired GD and/or GE.

The method can be implemented by, for example, incubating (culturing) an enantiomeric glycidyl ether with a wild-type yeast cell or a recombinant cell (yeast or any other host species listed herein) containing a nucleic acid sequence (e.g., a gene or a recombinant nucleic acid sequence) encoding a YEGH polypeptide, a crude extract from such cells, a semi-purified preparation of a YEGH polypeptide, or an isolated YEGH polypeptide, all of which exhibit epoxide hydrolase activity with chiral preference.

The strain of the yeast cell can be selected from the following genera: Arxula, Brettanomyces, Bullera, Bulleromyces, Candida, Cryptococcus, Debaryomyces, Dekkera, Exophiala, Geotrichum, Hormonema, Issatchenkia, Kluyveromyces, Lipomyces, Mastigomyces, Myxozyma, Pichia, Rhodosporidium, Rhodotorula, Sporidiobolus, Sporobolomyces, Trichosporon, Wingea, and Yarrowia.

Yeast strains innately capable of producing a polypeptide that converts or hydrolyses a range of different types of enantiomeric glycidyl ether to optically active (i.e. enantiopure or substantially enantiopure) equivalents and/or optically active associated diols include the following exemplary genera and species:

Arxula adeninivorans, Arxula terrestris, Brettanomyces bruxellensis, Brettanomyces naardenensis, Brettanomyces anomalus, Brettanomyces species (e.g. Unidentified species NCYC 3151), Bullera dendrophila, Bulleromyces albus, Candida albicans, Candida fabianii, Candida glabrata, Candida haemulonii, Candida intermedia, Candida magnoliae, Candida parapsilosis, Candida rugosa, Candida tenuis, Candida tropicalis, Candida famata, Candida kruisei, Candida sp. (new) related to C. sorbophila, Cryptococcus albidus, Cryptococcus amylolentus, Cryptococcus bhutanensis, Cryptococcus curvatus, Cryptococcus gastricus, Cryptococcus humicola, Cryptococcus hungaricus, Cryptococcus laurentii, Cryptococcus luteolus, Cryptococcus macerans, Cryptococcus podzolicus, Cryptococcus terreus, Debaryomyces hansenii, Dekkera anomala, Exophiala dermatitidis, Geotrichum spp. (e.g. Unidentified species UOFS Y-0111), Hormonema spp. (e.g. Unidentified species NCYC 3171), Issatchenkia occidentalis, Kluyveromyces marxianus, Lipomyces spp. (e.g. Unidentified species UOFS Y-2159), Lipomyces tetrasporus, Mastigomyces philipporii, Myxozyma melibiosi, Pichia anomala, Pichia finlandica, Pichia guillermondii, Pichia haplophila, Rhodosporidium lusitaniae, Rhodosporidium paludigenum, Rhodosporidium sphaerocarpum, Rhodosporidium toruloides, Rhodosporidium paludigenum, Rhodotorula araucariae, Rhodotorula glutinis, Rhodotorula minuta, Rhodotorula minuta var. minuta, Rhodotorula mucilaginosa, Rhodotorula philyla, Rhodotorula rubra, Rhodotorula spp. (e.g. Unidentified species NCYC 3193, UOFS Y-2042, UOFS Y-0448, UOFS Y-0139, UOFS Y-0560), Rhodotorula aurantiaca, Rhodotorula spp. (e.g. Unidentified species NCYC 3224), Rhodotorula sp. “mucilaginosa”, Sporidiobolus salmonicolor, Sporobolomyces holsaticus, Sporobolomyces roseus, Sporobolomyces tsugae, Trichosporon beigelii, Trichosporon cutaneum var. cutaneum, Trichosporon delbrueckii, Trichosporon jirovecii, Trichosporon mucoides, Trichosporon ovoides, Trichosporon pullulans, Trichosporon spp. (e.g. Unidentified species NCYC 3210, NCYC 3211, NCYC 3212, UOFS Y-0861, UOFS Y-1615, UOFS Y-0451, UOFS Y-0449, UOFS Y-2113), Trichosporon moniliiforme, Trichosporon montevideense, Wingea robertsiae, and Yarrowia lipolytica.

The yeast strain may be at least one yeast strain selected from the group consisting of the yeast species listed in Tables 2, 3, 4 and 5.

Cultivation in bioreactors (fermenters) of yeast strains expressing a YEGH polypeptide, or fragment thereof, (with the purpose of preparing yeasts stocks or for the enantioselective preparative methods of the invention) can be carried out under conditions that provide useful biomass and/or enzyme titer yields. Cultivation can be by batch, fed-batch or continuous culture methods. Useful cultivation conditions are dependent on the yeast strain used. General procedures for establishing useful growth conditions of yeasts, fungi and bacteria in bioreactors are known to those skilled in the art. The enantiomeric mixture of GE can be added directly to the culture. The concentration of the GE enantiomeric mixture in the reaction matrix can be at least equal to the soluble concentration of the GE enantiomeric mixture in water. The preferred GE level in the reaction matrix is greater than the solubility limit in the aqueous reaction medium thereby resulting in a two phase reaction system. The starting amount of GE added to the reaction mixture is not critical, provided that the concentration is at least equal to the solubility of the specific GE in the aqueous reaction medium. The GE can be metered out continuously or in batch mode to the reaction mixture. The relative proportions of the (R)- and (S)-glycidyl ether s in the mixture of enantiomers of the GE shown by the general formula (I) is not critical but it is advantageous for commercial purpose to employ a racemic form of the GE shown by the general formula (I). The GE can be added in a racemic form or as a mixture of enantiomers in different ratios.

The amount of the yeast cells, crude yeast cell extract, or partially purified or isolated polypeptide having GE enantioselective activity added to the reaction depends on the kinetic parameters of the specific reaction and the amount of GE that is to be hydrolysed. In the case of product inhibition, it can be advantageous to remove the formed GD from the reaction mixture or to maintain the concentration of the GD at levels that allow reasonable reaction rates. Techniques used to enhance enzyme and biomass yields include the identification of useful (or optimal) carbon sources, nitrogen sources, cultivation time, dilution rates (in the case of continuous culture) and feed rates, carbon starvation, addition of trace elements and growth factors to the culture medium, and addition of inducers (for example substrates or substrate analogs of the epoxide hydrolases) during cultivation. In the case of recombinant hosts, the conditions under which the promoters function workably for transcription of the gene encoding the polypeptide with epoxide hydrolase activity are taken into account. At the end of fermentation (culture), biomass and culture medium can be separated by methods known to one skilled in the art, such as filtration or centrifugation.

The processes are generally performed under mild conditions. For example, the reactions can be carried out at a pH from 5 to 10, preferably from 6.5 to 9, and most preferably from 7 to 8.5. The temperature for hydrolysis can be from 0 to 70° C., preferably from 0 to 50° C., most preferably from 4 to 40° C. It is also known that lowering of the temperature of the reaction can enhance enantioselectivity of an enzyme.

The reaction mixture can contain mixtures of water with at least one water-miscible solvents (e.g., water-miscible organic solvents). Preferably, water-miscible solvents are added to the reaction mixture such that epoxide hydrolase activity remains measurable. Water-miscible solvents are preferably organic solvents and can be, for example, acetone, methanol, ethanol, propanol, isopropanol, acetonitrile, dimethylsulfoxide, N,N-dimethylformamide, N-methylpyrrolidine, and the like.

The reaction mixture can also, or alternatively, contain mixtures of water with at least one water-immiscible organic solvent. Examples of water-immiscible solvents that can be used include, for example, toluene, 1,1,2-trichlorotrifluoroethane, methyl tert-butyl ether, methyl isobutyl ketone, dibutyl-o-phtalate, aliphatic alcohols containing 6 to 9 carbon atoms (for example hexanol, octanol), aliphatic hydrocarbons containing 6 to 16 carbon atoms (for example cyclohexane, n-hexane, n-octane, n-decane, n-dodecane, n-tetradecane and n-hexadecane or mixtures of the aforementioned hydrocarbons), and the like. Thus, the reaction mixture can include water with at least one water-immiscible organic solvent selected from the group consisting of toluene, 1,1,2-trichlorotrifluoroethane, methyl tert-butyl ether, methyl isobutyl ketone, dibutyl-o-phtalate, aliphatic alcohols containing 6 to 9 carbon atoms, and aliphatic hydrocarbons containing 6 to 16 carbon.

The reaction mixture can also contain surfactants (for example, Tween 80), cyclodextrins or any agent that can increase the solubility, selectively or otherwise, of the GE enantiomers in the aqueous reaction phase.

The reaction mixture can also contain a buffer. Buffers are known in the art and include, for example, phosphate buffers, Tris buffer, and HEPES buffers.

The production of the YEGH polypeptides, including functional fragments, can be, for example, as recited above in the section on Polypeptides and Polypeptide Fragments. Thus they can be made by production in a natural host cell, production in a recombinant host cell, or synthetic production. Recombinant production can be carried out in host cells of microbial origin. Preferred yeast host cells are selected from, but are not limited to, the genera Saccharomyces, Kluyveromyces, Hansenula, Pichia, Yarrowia and Candida. Preferred bacterial host cells include Escherichia coli, Agrobacterium species, Bacillus species and Streptomyces species. Preferred filamentous fungal host cells are selected from the group consisting of the genera Aspergillus, Trichoderma, and Fusarium. The production of the polypeptide can be, e.g., intra- or extra-cellular production and can be by, e.g., secretion into the culture medium.

In these fermentation reactions of the invention, the polypeptides (including functional fragments) can be immobilized on a solid support or free in solution. Procedures for immobilization of the yeast or preparation thereof include, but are not limited to, adsorption; covalent attachment; cross-linked enzyme aggregates; cross-linked enzyme crystals; entrapment in hydrogels; and entrapment into reverse micelles.

The progress of the reaction can be monitored by standard procedures known to one skilled in the art, which include, for example, gas chromatography or high-pressure liquid chromatography on columns containing chiral stationary phases. The GD formed can be removed from the reaction mixture at one or more stages of the reaction.

The reaction can be terminated when one enantiomer of the GE and/or GD is found to be in excess compared to the other enantiomer of the GE and/or GD. Preferably, the reaction is terminated when one enantiomer of a GE of general formula (I) and/or GD of general formula (II) is found to be in an enantiomeric excess of at least 90%. In a more preferred embodiment of the invention, the reaction is terminated when one enantiomer of a GE of general formula (I) and/or GD of general formula (II) is found to be in an enantiomeric excess of at least 95%. The reaction can be terminated by the separation (for example centrifugation, membrane filtration and the like) of the yeast, or a preparation thereof, from the reaction mixture or by inactivation (for example by heat treatment or addition of salts and/or organic solvents) of the yeast or polypeptide, or preparation thereof. Thus, the reaction can be stop for by, for example, the separation of the catalytic agent from the reactants and products in the mixture, or by ablation or inhibition of the catalytic activity, by techniques known to one skilled in the art.

The optically active GE and/or GD produced by the reaction can be recovered from the reaction mixture, directly or after removal of the yeast, or preparation thereof. Preferably, the process can include continuously recovering the optically active GE and/or GD produced by the reaction directly from the reaction mixture. Methods of removal of the optically active GE and/or GD produced by the reaction include, for example, extraction with an organic solvent (such as hexane, toluene, diethyl ether, petroleum ether, dichloromethane, chloroform, ethyl acetate and the like), vacuum concentration, crystallisation, distillation, membrane separation, column chromatography and the like.

Thus, the present invention provides an efficient process with economical advantages compared to other chemical and biological methods for the production, in high enantiomeric purity, of optically active GE of the general formula (I) and vicinal diol GD of the general formula (II) in the presence of a yeast strain having YEGH activity or a polypeptide having such activity.

### Yeast Epoxide Hydrolase Antibodies

The invention features antibodies that bind to yeast epoxide hydrolase polypeptides or fragments (e.g., antigenic or functional fragments) of such polypeptides. The polypeptides are preferably yeast epoxide polypeptides with enantioselective activity, and in particular those with glycidyl ether enantioselective activity (i.e., YEGH), e.g., those with SEQ ID NOs: 1, 2, 3, 4, 5, 6, 7, 8, or 9. The antibodies preferably bind specifically to yeast epoxide hydrolase polypeptides, i.e., not to epoxide hydrolase polypeptides of species other than yeast species. More preferably, they can bind specifically to yeast epoxide polypeptides with enantioselective activity, and in particular to YEGH polypeptides, e.g., those with SEQ ID NOs: 1, 2, 3, 4, 5, 6, 7, 8, or 9. They can moreover bind specifically to one or more of polypeptides with SEQ ID NOs: 1, 2, 3, 4, 5, 6, 7, 8, or 9.

Antibodies can be polyclonal or monoclonal antibodies; methods for producing both types of antibody are known in the art. The antibodies can be of any class (e.g., IgM, IgG, IgA, IgD, or IgE). They are preferably IgG antibodies. Moreover, polyclonal antibodies and monoclonal antibodies can be generated in, or generated from B cells from, animals any number of vertebrate (e.g., mammalian) species, e.g., humans, non-human primates (e.g., monkeys, baboons, or chimpanzees), horses, goats, camels, sheep, pigs, bovine animals (e.g., cows, bulls, or oxen), dogs, cats, rabbits, gerbils, hamsters, guinea pigs, rats, mice, birds (such as chickens or turkeys), or fish.

Recombinant antibodies specific for YEGH polypeptides, such as chimeric monoclonal antibodies composed of portions derived from different species and humanized monoclonal antibodies comprising both human and non-human portions, are also encompassed by the invention. Such chimeric and humanized monoclonal antibodies can be produced by recombinant DNA techniques known in the art, for example, using methods described in Robinson et al., International Patent Publication PCT/US86/02269; Akira et al., European Patent Application 184,187; Taniguchi, European Patent Application 171,496; Morrison et al., European Patent Application 173,494; Neuberger et al., PCT Application WO 86/01533; Cabilly et al., U.S. Pat. No. 4,816,567; Cabilly et al., European Patent Application 125,023; Better et al. (1988) Science 240, 1041-43; Liu et al. (1987) J. Immunol. 139, 3521-26; Sun et al. (1987) PNAS 84, 214-18; Nishimura et al. (1987) Canc. Res. 47, 999-1005; Wood et al. (1985) Nature 314, 446-49; Shaw et al. (1988) J. Natl. Cancer Inst. 80, 1553-59; Morrison, (1985) Science 229, 1202-07; Oi et al. (1986) BioTechniques 4, 214; Winter, U.S. Pat. No. 5,225,539; Jones et al. (1986) Nature 321, 552-25; Veroeyan et al. (1988) Science 239, 1534; and Beidler et al. (1988) J. Immunol. 141, 4053-60.

Also useful for the invention are antibody fragments and derivatives that contain at least the functional portion of the antigen-binding domain of an antibody that binds to a YEGH polypeptide. Antibody fragments that contain the binding domain of the molecule can be generated by known techniques. Such fragments include, but are not limited to: F(ab′)₂ fragments that can be produced by pepsin digestion of antibody molecules; Fab fragments that can be generated by reducing the disulfide bridges of F(ab′)₂ fragments; and Fab fragments that can be generated by treating antibody molecules with papain and a reducing agent. See, e.g., National Institutes of Health, 1 Current Protocols In Immunology, Coligan et al., ed. 2.8, 2.10 (Wiley Interscience, 1991). Antibody fragments also include Fv fragments, i.e., antibody products in which there are few or no constant region amino acid residues. A single chain Fv fragment (scFv) is a single polypeptide chain that includes both the heavy and light chain variable regions of the antibody from which the scFv is derived. Such fragments can be produced, for example, as described in U.S. Pat. No. 4,642,334, which is incorporated herein by reference in its entirety. The antibody can be a “humanized” version of a monoclonal antibody originally generated in a different species.

The above-described antibodies can be used for a variety of purposes including, but not limited to, YEGH polypeptide purification, detection, and quantitative measurement.

The following examples serve to illustrate, not limit, the invention.

### EXAMPLES

### Example I

### Materials and Methods

### Determination of Concentrations and Enantiomeric Excesses

In the examples, quantitative determinations of the compounds and determination of enantiomeric excesses were carried out by GC and HPLC. Gas chromatography (GLC) was performed on a Hewlett-Packard 6890 gas chromatograph equipped with FID detector and using H₂ as carrier gas. HPLC was performed on a Hewlett-Packard 1050 liquid chromatograph equipped with a UV detector. Chiral analysis of glycidyl ethers was done as follows:

| Compound | Chiral column | Conditions | Retention time (min) |
|---|---|---|---|
| Phenyl glycidyl ether: | HPLC | hexane/2-propanol | (R)-: 10.5; (S)-: 15.4 |
| | Chiralcel OD | 8:2; 1 ml/min | |
| 3-phenoxypropane-1,2- | HPLC | hexane/2-propanol | (R)-: 25.10; (S)-: 47.05 |
| diol | Chiralcel OD | 9:1; 1 ml/min | |
| Benzyl glycidyl ether | HPLC | hexane/2-propanol | (S)-: 9.3; (R)-: 10.1 |
| | Chiralcel OD | 85:15; 1 ml/min | |
| Furfuryl glycidyl ether | HPLC | hexane/ethanol | (S)-: 20.5; (R)-: 21.5 |
| | Chiralcel OB-H | 95:5; 1 ml/min | |
| Glycidyl isopropyl ether | GC | 60° C. isotherm (head | (S): 9.6; (R)-: 9.9 |
| | β-Dex 120 (Supelco) | pressure 10 psi) | |
| Naphtyl glycidyl ether | HPLC | Hexane/ethanol 8:2; | (R)-: 24.5; (S)-: 26.2 |
| | Chiralcel OB-H | 1.2 ml/min | |
| Glycidyl tosylate | HPLC | hexane/2-propanol | (R)-: 29.5; (S)-: 30.5 |
| | Chiralpak AD-H | 95:5; 1.4 ml/min | |
| Glycidyl-4-nitrobenzoate | HPLC | hexane/2-propanol | (S)-: 25.9; (R)-: 26.7 |
| | Chiralpak AD-H | 95:5; 1.4 ml/min | |

### Synthesis of Glycidyl Ether Substrates

The glycidyl ethers that were used to illustrate the use of the different yeast strains to prepare optically active glycidyl ethers (GE) and vicinal diols (GD) from enantiomeric mixtures of glycidyl ethers represented by the general formula (I) is given in Scheme I.

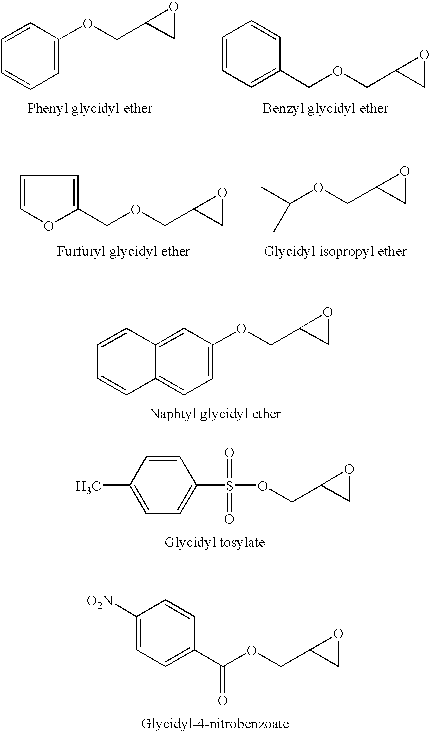

All GE substrates used were commercially available, with the exception of benzyl glycidyl ether and naphtyl glycidyl ether.

Benzyl glycidyl ether was synthesized by addition of epichlorohydrin to benzylalcohol as follows:

A mixture of benzyl alcohol (1.401, 13.53 mol) and epichlorohydrin (1.161, 14.88 mol) was placed in a 10 l mechanically stirred baffled reactor with efficient cooling. The mixture was cooled to ˜5° C., treated with tetra-n-butylammonium iodide (99.94 g, 0.27 mol), followed by dosing of 50% (w/v) aqueous sodium hydroxide solution (7.5 l, 93.75 mol) in four portions over approximately 1 h. No substantial exotherm was noted. The dense emulsion formed on agitation at 600 rpm was left at this temperature for 2 h, then allowed to gradually warm to room temperature over 18 h. The mixture was then extracted with dichloromethane (2×2.5 l) and the solvent removed until about 2 l remained, after which MgSO₄ was added to the stirred solution. Filtration and further concentration afforded about 1.6 l of orange-brown oil. Two cycles of distillation (95-97° C./0.4 mmHg) afforded a clear oil, benzyl glycidyl ether (1.13 kg, 51%). δ_{H} (200 MHz, CDCl₃) 7.41-7.26 (5H, m, aryl H), 4.65 (d, 1H, PhCHₐH_{b}, J 12.0), 4.58 (d, 1H, PhCHₐH_{b}, J 12.0), 3.79 (dd, 1H, OCHₐH_{b}, J 11.6 and 3.2), 3.47 (dd, 1H, OCHₐH_{b}, J 11.4 and 5.6), 3.26-3.14 [m, 1H, CH₂CH(O)], 2.82 [˜t, 1H, CHCHₐH_{b}(O), J 4.6] and 2.64 [dd, 1H, CHCHₐH_{b}(O), J 5.2 and 2.8].

Naphtyl glycidyl ether was synthesised as follows:

A mixture of 2-naphthol (5.014 g, 34.780 mmol) in epichlorohydrin (35 cm³) was treated with solid potassium carbonate (10.407 g, 75.298 mmol) in the presence of tetra-n-butylammonium iodide (0.192 g, 0.521 mmol) as phase-transfer catalyst. The mixture was stirred for 18 h at room temperature, after which it was diluted with 50 cm³ each of dichloromethane and water. Extraction with dichloromethane, drying (MgSO₄) and concentration afforded an orange oil. This was distilled (180-190° C./3 mmHg) to afford a clear oil that crystallised on standing. The yellow tacky solid was recrystallised with difficulty from ethyl acetate/hexane to afford white needles of 2-naphtyl glycidyl ether (3.525 g, 51%). δ_{H} (200 MHz, CDCl₃) 7.80-7.59 and 7.58-7.06 (7H, 2×m, aryl H), 4.35 (1H, dd, OCHₐH_{b}, J 11.2 and 3.4), 4.09 (1H, dd, OCHₐH_{b}, J 11.2 and 5.6), 3.52-3.29 [m, 1H, CH₂CH(O)], 2.94 [˜t, 1H, CHCHₐH_{b}(O), J 5.0] and 2.81 [dd, 1H, CHCHₐH_{b}(O), J 4.8 and 2.8].

Diol standards were prepared by acid hydrolysis of the corresponding glycidyl ethers.

### Preparation of Frozen Yeast Cells for Screening

Yeasts were grown at 30° C. in 1 L shake-flask cultures containing 200 ml yeast extract/malt extract (YM) medium (3% yeast extract, 2% malt extract, 1% peptone w/v) supplemented with 1% glucose (w/v). At late stationary phase (48-72 h) the cells were harvested by centrifugation (10 000 g, 10 min, 4° C.), washed with phosphate buffer (50 mM, pH7.5), centrifuged and frozen in phosphate buffer containing glycerol (20%) at −20° C. as 20% (w/v) cell suspensions. The cells were stored for several months without significant loss of activity.

### Isolate Screening

Glycidyl ether (GE) substrate (10 μl of a 1M stock solution in EtOH) was added to a final concentration of 20-50 mM to 100-500 μl cell suspension (20-50% w/v) in phosphate buffer (50 mM, pH 7.5). The reaction mixtures were incubated at 25° C. for 1-5 hours. The reaction mixtures were extracted with EtOAc or hexane (equal volume) and centrifuged. GD formation was evaluated by TLC (silica gel Merck 60 F₂₅₄). Compounds were visualized by spraying with vanillin/conc. H₂SO₄ (5 g/l). Reaction mixtures that showed substantial GD formation were evaluated for asymmetric hydrolysis of the GE by chiral GLC or HPLC analysis. Some reactions were repeated over longer or shorter times and with more dilute cell suspensions (10% w/v) in order to analyse the reactions at suitable conversions.

### General Procedure for the Hydrolysis of Glycidyl Ethers

Frozen cells were thawed, washed with phosphate buffer (50 mM, pH 7.5) and resuspended in buffer. Cell suspensions (10 ml, 20% or 50% w/v) were placed in 20 ml glass bottles with screw caps fitted with septa. The substrate (100 or 250 μl of a 2M (v/v) stock solution in ethanol) was added to final concentrations of 20 mM or 50 mM. The mixtures were agitated on a shaking water bath at 30° C. The course of the bioconversions of the GE was followed by withdrawing samples (500 μl) at appropriate time intervals. Samples were extracted with 300 μl EtOAc or hexane. After centrifugation (3000×g, 2 min), the organic layer was dried over anhydrous MgSO₄ and the products analyzed by chiral GLC or HPLC.

### Determination of the Absolute Configuration of Glycidyl Ethers and Residual Diols

Absolute configurations were deduced by the elution order of the GE enantiomers on chiral HPLC columns as reported in literature (Xu et. al., 2004).

### Yeast Strains

Yeast strains with “Jen” and numerical screen numbers were obtained from the Yeast Culture Collection of the University of the Free State. Yeast strains with screen numbers donated “AB” or “Car” or “Alf” or “Poh” were isolated from soil from specialised ecological niches that were selected based on our hypothesis that selectivity for specific classes of epoxides in microorganisms may be determined by environmental factors such as terpene-rich environments or highly contaminated soil. “AB” and “Alf” strains were isolated from Cape Mountain fynbos, an ecological environment unique to South Africa, “Car” strains were isolated from soil under pine trees, and “Poh” strains from soil contaminated by high concentrations of cyanide. These new isolated were subsequently deposited at the Yeast Culture Collection of the Free State and assigned UOFS numbers.

Cloning and Overexpression of Wild Type Yeast Epoxide Hydrolases in Yarrowia lipolytica as Production Host Under the Control of Different Promoters

### 1. Vectors, Strains and Primers (Table 1)

The following features are common to all the E. coli/Y. lipolytica auto-cloning integrative vectors used:
LIP2 terminator
Zeta regions
Kanamycin resistance for E. coli selection
mono-copy auto cloning vectors (pINA 1311, pINA 1313, pINA 3313) with a fully functional selection marker gene carries the fully functional ura3d1 allele from the URA3 selection marker gene
multi-copy auto cloning vectors (pINA 1291, pINA 1293, pINA 3293) with a defective selection marker gene (copy number amplification) carries the defective ura3d4 allele from the URA3 selection marker gene

TABLE 1
Vectors, strains and primers

| | |
|---|---|
| Description | Cloning |

| | | | | sites | |
|---|---|---|---|---|---|
| | | Selection | Targeting | Upstream/ | Reference/ |
| Vectors | Promoter | marker | sequence | downstream | Origin |
| pINA1291 = | hp4d | ura3d4 | none | Pm/I (blunt)/ | Nicaud et al |
| pYLHmA | | | | BamHI, KpnI, | (2002) |
| | | | | AvrII | |
| pINA1311 | hp4d | ura3d1 | none | Pm/I (blunt)/ | Nicaud et al |
| (1291) | | | | BamHI, KpnI, | (2002) |
| pYLHsA | | | | AvrII | |
| pINA 1293 | hp4d | ura3d4 | LIP2 | XmnI (in pro)/ | Nicaud et al |
| pYLHmL | | | prepro | BamHI, KpnI, | (2002) |
| | | | | AvrII | |
| pINA 1313 | hp4d | ura3d1 | LIP2 | XmnI (in pro)/ | Nicaud et al |
| (1293) | | | prepro | BamHI, KpnI, | (2002) |
| pYLHsL | | | | AvrII | |
| pYL3313 | TEF | ura3d1 | none | XmnI (in pro)/ | This study |
| (1313) | | | | BamHI, KpnI | |
| pYLTsA | | | | AvrII | |
| pYL3293 | TEF | ura3d4 | none | XmnI (in pro)/ | This study |
| (1293) | | | | BamHI, KpnI | |
| pYLTmA | | | | AvrII | |

| Host | | Reference/ |
|---|---|---|
| Strain | Description | Origin |
| Yarrowia | MATA, ura3-302, uxpr2-322, axp1-2 | CLIB882 |
| lipolytica | (deleted for both extracellular | |
| Po1h | proteases and growth on sucrose | |

| Primers | Sequence | Specifications |
|---|---|---|
| YL-fwd | 5′-GGA GTT CTT CGC CCA C-3′ | amplification of expression |
| | | cassette between NotI sites |
| YL-rev | 5′-GAT CCC CAC CGG AAT TG-3′ | amplification of expression |
| | | cassette between NotI sites |
| pINA-1 | 5′-CAT ACA ACC ACA CAC ATC CA-3′ | pYLHmA fwd primer |
| pINA-2 | 5′-TAA ATA GCT TAG ATA CCA CAG-3′ | pYLTsA/pYLHmA rev primer |
| pINA-3 | 5′-CTC TCT CTC CTT GTC AAC T-3′ | pYLTsA fwd primer |

### 2. Transformants (Multi-Copy and Single-Copy)

| Transformants | Gene origin |
|---|---|
| TEF promoter | Vector: pYL3313 (1313) = (pYLTsA) |
| YL 23 TsA | Rhodotorula mucilaginosa NCYC 3190 |
| YL 25 TsA | Rhodotorula araucariae NCYC 3183 |
| YL 46 TsA | Rhodosporidium toruloides UOFS Y-0471 |
| YL 692 TsA | Rhodosporidium paludigenum NCYC 3179 |
| YL 777 TsA | Cryptococcus neoformans var neoformans |
| YL 1 TsA | Rhodosporidium toruloides NCYC 3181 |
| YL Car 54 TsA | Cryptococcus curvatus NCYC 3158 |
| YL Po1h-1 TsA | Yarrowia lipolytica Po1h |
| YL Po1h-2 TsA | Yarrowia lipolytica Po1h |
| YL Jen 42-2 TsA | Yarrowia lipolytica UOFS Y-1138 |
| YL Jen 46-2 TsA | Yarrowia lipolytica NCYC 3229 |
| hp4d promoter | Vector: pINA1291 = (pYLHmA) |
| YL 1 HmA | Rhodosporidium toruloides NCYC 3181 |
| YL 23 HmA | Rhodotorula mucilaginosa NCYC 3190 |
| YL 25 HmA | Rhodotorula araucariae NCYC 3183 |
| YL 46 HmA | Rhodosporidium toruloides UOFS Y-0471 |
| YL 692 HmA | Rhodosporidium paludigenum NCYC 3179 |
| YL 777 HmA | Cryptococcus neoformans var neoformans |

### 3. Vector Preparation

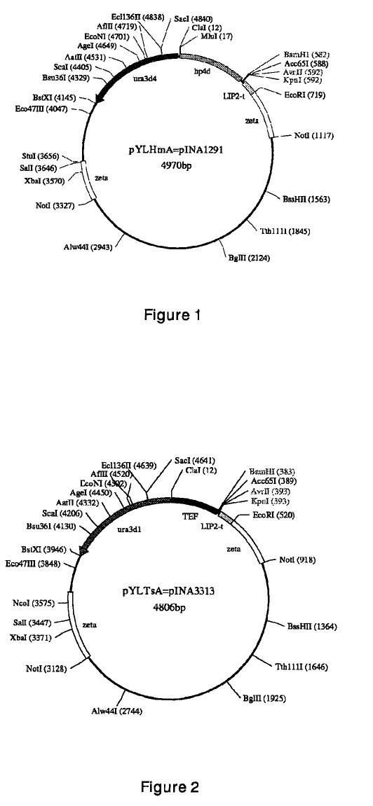

pINA1291 (FIG. 1) was received from Dr Madzak of labo de Génétique, INRA, CNRS. This was renamed pYLHmA (Yarrowia Lipolytica expression vector, with Hp4d promoter, multi-copy integration selection, A=no secretion signal)

pINA3313 (FIG. 2) was prepared in this study. This was renamed pYLTsA (Yarrowia Lipolytica expression vector, with TEF promoter, single-copy integration selection, A=no secretion signal).

To prepare the vectors for ligation with an epoxide hydrolase gene (or other insert to be expressed in Y. lipolytica), DNA was digested with BamHI and AvrII, and dephosphorylated using commercial Calf Intestinal Alkaline Phosphatase.

### 4. Insert Preparation

Total RNA was isolated from selected yeast strain cells and messenger RNA (mRNA) was purified from it. The mRNA was used as a template to synthesise complementary DNA (cDNA) using reverse transcriptase. The cDNA was then used as a template for Polymerase Chain Reaction (PCR) using appropriate primers. PCR primers were selected by repeated experimentation using multiple test primers for each yeast strain, the sequences of which were based on previously described epoxide hydrolase sequences from a variety of species. The nucleotide sequences of the forward and reverse primers used to generate cDNA coding sequences from mRNA from seven different yeast strains with appropriate restriction enzyme recognition sites at their termini are shown below. Restriction enzyme recognition sequences are underlined and the relevant restriction enzymes are shown in parentheses.

| | | |
|---|---|---|
| Strain | Forward primer | Reverse primer |

| | | |
|---|---|---|
| R. toruloides NCYC | GTGGATCCATGGCGACACACA | GACCTAGGCTACTTCTCCCACA |
| 3181 (#1) | (BamHI) | (BlnI) |
| R. toruloides UOFS | | |
| Y-0471 (#46) | | |
| C. curvatus NCYC | | |
| 3158 Car 054 | | |
| R. araucariae NCYC | GATTAATGATCAATGAGCGAGCA | GACCTAGGTCACGACGACAG |
| 3183 (#25) | (BclI) | (BlnI) |
| R. paludigenum NCYC | GTGGATCCATGGCTGCCCA | GAGCTAGCTCAGGCCTGG |
| 3179 (#692) | (BamHI) | (NheI) |
| R. mucilaginosa NCYC | GTATATCTATGCCCGCCCGCT | GACCTAGGCTACGATTTTTGCT |
| 3190 (#23) | (BglII) | (BlnI) |
| Y. lipolytica NCYC | GCAGATCTATGTCATCACTCG | GACCTAGGCTACAACTTCGACG |
| 3228 (Jen 46-2) | (BglII) | (BlnI) |
| Debaromyces hansenii | GTGGATCCATGATGCAAGG | GACCTAGGCTAAGGATATT |
| NCYC 3167 (#113) | (BamHI) | (BlnI) |
| Filobasidium | GAGGATCCATGTCGTATTCAGA | GAGCTAGCTCAGTAATTACCTTTG |
| neoformans | (BamHI) | (Nhe)I |
| (#777) | | |

Each PCR reaction contained 200 M dNTPs, 250 nM of each primer, 2 mM of MgCl₂, cDNA and 2.5 U of Taq polymerase in a 50 μl reaction volume. The PCR profile used was: 95° C. for 5 minutes, followed by 30 cycles of: 95° C.—1 min, 50° C.—1 min, 72° C.—2 min, then a final extension of 72° C. for 10 minutes. The PCR products were purified and digested with the restriction enzymes whose recognition sites are engineered at the end of the primers. The cDNA fragment was cloned into a vector and sequenced for confirmation.

Coding sequences to be inserted in either pYLHmA or pYLTsA were prepared with BamHI and AvrII at their termini. The above PCR primers were designed with these restriction sites, unless the sites were also present in the gene to be inserted. If this occurred, appropriate compatible restriction enzymes were selected. PCR template DNA was either the insert cloned into a different vector, or cDNA synthesized from the original host organism. PCR reactions consisted of 200 M dNTP's, 250 nM each primer, 1×Taq polymerase buffer, and 2.5 units Taq polymerase per 100 l reaction. The amplification programme used was: 95° C. for 5 minutes, 30 cycles of 95° C. for 1 minute, 50° C. for 1 minute, and 72° C. for 2 minutes, followed by a single duration at 72° C. for 10 minutes.

PCR products were purified and digested with the relevant restriction enzymes. The digested DNA was subsequently repurified and was ready for ligation into the prepared vector.

5. Preparation of pYLHmA or pYLTsA Constructs

Vector and insert were ligated at pmol end ratios of 3:1-10:1 (insert:vector), using commercial T4 DNA Ligase. Ligations were electroporated into any laboratory strain of Escherichia coli, using the Bio-Rad GenePulser, or equivalent electroporator. Transformants were selected on LM media (10 g/l yeast extract, 10 g/l tryptone, 5 g/l NaCl), supplemented with kanamycin (50 g/ml). Transformants were selected based on restriction enzyme digests of purified plasmid DNA.

6. Yarrowia lipolytica Transformation

### 6.1.1. Preparation of DNA—Method 1

Digestion of the pINA-series of plasmids with NotI resulted in the release of a bacterial DNA-free expression cassette, containing the ura3d4 (pYLHmA) or the ura3d1 (pYLTsA) marker gene and the promoter-gene-terminator.

Scaled-up quantities of each plasmid were isolated. NotI was used to restrict the plasmid DNA, and the digested DNA was run on an agarose gel. NotI digests resulted in generation of the bacterial fragment of the plasmid as a band at 2210 bp, and the expression cassette as a band of 2760 bp+size of insert (pYLHmA) or 2596 bp+size of insert (pYLTsA). The expression cassette fragments were excised from the gel and purified from the agarose. The purified fragment was used for transformation of Y. lipolytica Po1 h.

### 6.1.2. Preparation of DNA—Method 2

Primers YL-Fwd and YL-Rev were used to amplify the expression cassette. PCR reactions consisted of 200 M dNTP's, 250 pmol each primer, 1×Taq polymerase buffer and 2.5 units Taq polymerase per 100 l reaction. The amplification programme used was 95° C. for 5 minutes, 30 cycles of 95° C. for 1 minute, 50° C. for 1 minute, and 72° C. for 3½ minutes, followed by 72° C. for 10 minutes. The PCR product was purified from the PCR reaction mix and used for transformation of Y. lipolytica Po1 h.

### 6.1.3. Preparation of Carrier DNA

DNA from salmon testes was made up as a 10 mg/ml stock in TE (10 mM Tris-HCl, pH 8.0, 1 mM EDTA) and sonicated to produce in fragments that range from approximately 15 kb to 100 bp, with most fragments in a range of 6 to 10 kb. The DNA was denatured by boiling. Aliquots are stored at −20° C.

6.1.4. Transformation of Yarrowia lipolytica with pYLHmA or pYLTsA

An adaptation of the method of Xuan et al (1988) was used for the transformation of Y. lipolytica Po1h. The yeast was inoculated into 50 ml YPD (10 g/l yeast extract, 20 g/l peptone, 20 g/l glucose) The culture was incubated at 30° C., 220 rpm until cell densities of 8×10⁷-2×10⁸ cells/ml were reached. The entire culture was harvested, the pellet resuspended in 10 ml TE and reharvested. 1 ml TE+0.1 M LiOAc was used to resuspend the pellet and the culture was incubated at 28° C. in a ProBlot Jr (Labnet) hybridisation oven, set at 4 rpm (or similar incubator) for 1 hour. Transformation mixes were set up with 0.5-2 g of transforming DNA+5 g of carrier DNA with 100 l of treated cells.

Each mix was set up in a 1.5 ml microfuge tube, and incubated in a 28° C. heating block for 30 minutes. 7 volumes of PEG reagent (40% PEG 4,000, 0.1 M LiOAc, 10 mM Tris, 1 mM EDTA, pH 7.5, filter-sterilised) were added to each, mixed carefully and incubated at 28° C. for a further 1 hour. The tubes were transferred to a 37° C. heating block for 15 minutes, and then pelleted for 1 minute at 13,000 rpm and the pellets carefully resuspended in 100 l dH₂O. The transformations were plated on Y. lipolytica selective plates (17 g/l Difco yeast nitrogen base without amino acids and without (NH₄)₂SO₄, 20 g/l glucose, 4 g/l NH₄Cl, 2 g/l casamino acids, 300 mg/l leucine) and incubated at 28° C. Colonies appearing on the selective plates after 3-7 days were transferred onto fresh plates and regrown.

6.1.5. Confirmation of Integration of pYLHmA or pYLTsA

Colonies that grow on the newly-streaked selective plates were inoculated into 5 ml of YPD and grown at 30° C., 200 rpm for 24-48 hours. A small-scale genomic DNA isolation was performed.

PCR was performed using this genomic DNA as template, with either pINA-1 and pINA-2 as primers (transformants with pYLHmA), or pINA-3 and pINA-2 (pYLTsA). Each PCR reaction contained 200 M dNTPs, 250 nM of each primer, 2 mM of MgCl₂, genomic DNA and 2.5 U/50 l of Taq polymerase. The PCR profile was as described above in 6.1.2. These primer sets should result in products the size of the inserted genes.

### Example 2

### Selection of yeasts that are able to produce optically active (R)-phenyl glycidyl ether (phenoxypropylene oxide) and (S)-3-phenoxy-1,2-propanediol from (±)-phenyl glycidyl ether

Yeasts were cultivated, harvested and frozen as described above. The racemic GE was added and the screening was performed as described above. Strains with the highest activities as judged by TLC from diol formation were subjected to chiral HPLC analysis as described above. The strains with E-values >2 are given as samples 1-55 in Table 2. E-values were calculated using the following formula:

$$E = \frac{ln [ \frac{{ee}_{p} ( 1 - {ee}_{s} )}{( eep + ees )} ]}{ln [ \frac{{ee}_{p} ( 1 + {ee}_{s} )}{( eep + ees )} ]} where \begin{matrix}{ee}_{s} = substrate enantiomeric excess \\ {ee}_{p} = product enantiomeric excess\end{matrix}$$

All the yeast strains referred to in this and the following examples are kept and maintained at the University of the Free State (UFS), Department of Microbial, Biochemical and Food Biotechnology, Faculty of Natural and Agricultural Sciences, P.O. Box 339, Bloemfontein 9300, South Africa (Tel +27 51 401 2396, Fax +27 51 444 3219) and are readily identified by the yeast species and culture collection number as indicated. Representative examples of strains belonging to the different species have been deposited under the Budapest Treaty at National Collection of Yeast Cultures (NCYC), Institute of Food Research Norwich Research Park Colney, Norwich NR4 7UA, U.K. (Tel: +44-(0)1603-255274 Fax: +44-(0)1603-458-414 Email: ncyc@bbsrc.ac.uk) and are readily identified by the yeast species and culture collection accession number as indicated. The samples deposited with the NCYC are taken from the same deposit maintained by the South African Council for Scientific and Industrial Research (CSIR) since prior to the filing date of this application. The deposits will be maintained without restriction in the NCYC depository for a period of 30 years, or 5 years after the most recent request, or for the effective life of the patent, whichever is longer, and will be replaced if the deposit becomes non-viable during that period. Samples of the yeast strains not deposited at NCYC will be made available upon request on the same basis and conditions of the Budapest Treaty.

TABLE 2
Yeast strains from different genera that hydrolyse phenyl glycidyl ether
enantioselectively^{a}

| | | | | | (R)- | (S)- | |
|---|---|---|---|---|---|---|---|
| | | | Culture | Time | epoxide | diol | |
| | SNO | Genus | Collection nr. | (min) | eeₛ (%) | eeₚ (%) | E |
| 1 | 693 | Arxula terrestris | NCYC 3148 | 180 | 25.3 | Nd | Nd |
| 2 | Jen 07 | Arxula terrestris | UOFS Y-1225 | 180 | 35.4 | Nd | Nd |
| 3 | 752 | [symbol] | UOFS Y-1041 | 180 | 80.5 | 82.4 | 25.5 |
| 4 | 678 | Candida magnoliae | UOFS Y-1297 | 60 | 57.6 | 51.6 | 5.5 |
| 5 | 708 | Candida rugosa | NCYC 3155 | 180 | 20.6 | 60.5 | 5.0 |
| 6 | POH 29 | Candida sp. (new) rel to C. sorbophila | NCYC 3217 | 180 | 33.8 | 63.3 | 6.1 |
| 7 | 34 | Candida tenuis | UOFS Y-1328 | 180 | 27.9 | 60.5 | 5.3 |
| 8 | Car 054 | [symbol] | NCYC 3158 | 180 | 68.5 | 87.7 | 31.3 |
| 9 | Car 014 | [symbol] | UOFS Y-2225 | 180 | 53.3 | 84.5 | 20.2 |
| 10 | TT 05 | Cryptococcus humicola | UOFS Y-0571 | 180 | 35.9 | 80.3 | 13.0 |
| 11 | Car 137 | Cryptococcus humicola | UOFS Y-2254 | 180 | 89.4 | 68.3 | 15.6 |
| 12 | Car 220a | [symbol] | UOFS Y-2262 | 180 | 74.0 | 80.6 | 20.5 |
| 13 | Car 400 | [symbol] | UOFS Y-2263 | 180 | 80.4 | 78.5 | 20.3 |
| 14 | 93 | Debaryomyces hansenii | NCYC 3169 | 180 | 5.1 | 79.5 | 9.2 |
| 15 | 105 | Debaryomyces hansenii | UOFS Y-0608 | 180 | 11.4 | 81.8 | 11.1 |
| 16 | 17 | Debaryomyces hansenii | UOFS Y-0492 | 180 | 18.0 | Nd | Nd |
| 17 | 45 B | Lipomyces sp. (course) | UOFS Y-2159 B | 180 | 21.0 | 88.0 | 19.3 |
| 18 | 45 A | Lipomyces sp. (smooth) | UOFS Y-2159 A | 180 | 25.4 | 20.5 | 1.9 |
| 19 | 466 | Mastigomyces philipporii | UOFS Y-1139 | 180 | 6.5 | 16.1 | 1.5 |
| 20 | 702 | Pichia guillermondii | NCYC 3175 | 180 | 26.7 | 81.6 | 12.8 |
| 21 | 706 | Pichia guillermondii | UOFS Y-0057 | 180 | 22.7 | Nd | Nd |
| 22 | 674 | Pichia guillermondii | UOFS Y-1030 | 180 | 48.1 | 85.0 | 19.9 |
| 23 | 675 | Pichia guillermondii | UOFS Y-1033 | 180 | 59.9 | 82.9 | 19.6 |
| 24 | 673 | Pichia haplophila | NCYC 3177 | 180 | 16.2 | 70.8 | 6.8 |
| 25 | Car 118 | Rhodosporidium toruloides | NCYC 3182 | 180 | 37.3 | 86.2 | 19.5 |
| 26 | Car 006 | [symbol] | UOFS Y-2223 | 180 | 64.6 | 87.4 | 29.0 |
| 27 | Car 020 | [symbol] | UOFS Y-2226 | 180 | 46.2 | 88.6 | 26.1 |
| 28 | Car 038 | Rhodosporidium toruloides | UOFS Y-2228 | 180 | 37.1 | 86.2 | 19.4 |
| 29 | Car 052 | [symbol] | UOFS Y-2230 | 180 | 81.8 | 90.2 | 49.4 |
| 30 | Car 059 | [symbol] | UOFS Y-2231 | 180 | 36.1 | 89.9 | 26.8 |
| 31 | Car 076 | [symbol] | UOFS Y-2236 | 180 | 61.8 | 89.7 | 34.6 |
| 32 | Car 078 | [symbol] | UOFS Y-2240 | 180 | 65.5 | 86.7 | 27.5 |
| 33 | Car 092 | Rhodosporidium toruloides | UOFS Y-2241 | 180 | 56.3 | 82.4 | 18.2 |
| 34 | Car 093 | [symbol] | UOFS Y-2242 | 180 | 86.4 | 83.3 | 30.3 |
| 35 | Car 099 | Rhodosporidium toruloides | UOFS Y-2243 | 180 | 91.1 | 67.8 | 16.0 |
| 36 | Car 100 | [symbol] | UOFS Y-2245 | 180 | 78.4 | 86.4 | 32.7 |
| 37 | Car 108 | [symbol] | UOFS Y-2247 | 180 | 46.9 | 87.8 | 24.5 |
| 38 | Car 120 | [symbol] | UOFS Y-2249 | 180 | 49.8 | 87.1 | 23.8 |
| 39 | Car 121 | [symbol] | UOFS Y-2250 | 180 | 77.5 | 87.2 | 34.2 |
| 40 | Car 126 | [symbol] | UOFS Y-2251 | 180 | 42.4 | 87.2 | 22.2 |
| 41 | Car 200 | [symbol] | UOFS Y-2256 | 180 | 64.0 | 92.9 | 52.7 |
| 42 | EP 230 | [symbol] | NCYC 3185 | 180 | 27.8 | 91.8 | 30.8 |
| 43 | Car 022 | [symbol] | UOFS Y-2227 | 180 | 42.3 | 92.4 | 38.1 |
| 44 | Car 060 | [symbol] | UOFS Y-2232 | 180 | 39.6 | 87.9 | 23.0 |
| 45 | Car 061 | [symbol] | UOFS Y-2233 | 180 | 67.3 | 83.7 | 22.7 |
| 46 | 714 | Rhodotorula minuta | NCYC 3187 | 180 | 31.8 | 83.3 | 15.0 |
| 47 | 690 | Rhodotorula sp. nearest | UOFS Y-0125 | 180 | 48.5 | 80.8 | 15.1 |
| | | minuta | | | | | |
| 48 | Jen 29 | [symbol] | NCYC 3197 | 180 | 100.0 | 72.3 | 84.6 |
| 49 | 22 | Trichosporon jirovecii | NCYC 3204 | 180 | 78.6 | Nd | Nd |
| 50 | 14 | [symbol] | NCYC 3205 | 180 | 56.2 | 92.4 | 44.5 |
| 51 | 223 | Trichosporon mucoides | UOFS Y-0116 | 180 | 47.6 | Nd | Nd |
| 52 | 231 | [symbol] sp. | NCYC 3210 | 60 | 88.3 | 91.5 | 66.1 |
| 53 | 225 | [symbol] sp. | NCYC 3211 | 180 | 7.1 | 100.0 | >200 |
| 54 | 224 | [symbol] sp. | UOFS Y-0449 | 180 | 33.5 | 100.0 | >200 |
| 55 | TT 33 | Yarrowia lipolytica | UOFS Y-0647 | 180 | 36.2 | 77.8 | 11.4 |

^{a}Reaction conditions: 50 mM glycidyl ether, 50% cells (w/v) in phosphate buffer (50 mM, pH 7.5)

### Examples 3

### Hydrolysis of (±)-Phenyl Glycidyl Ether by Selected Wild Type Yeasts to Produce Optically Active (R)-Phenyl Glycidyl Ether and the Corresponding (S)-Diol

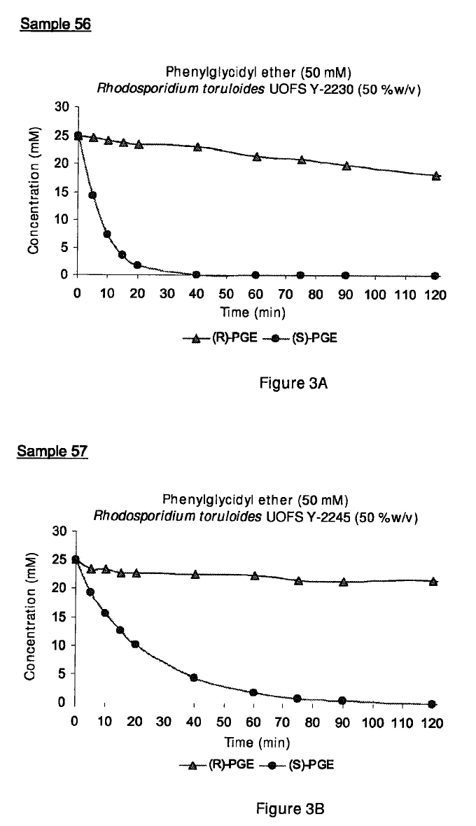

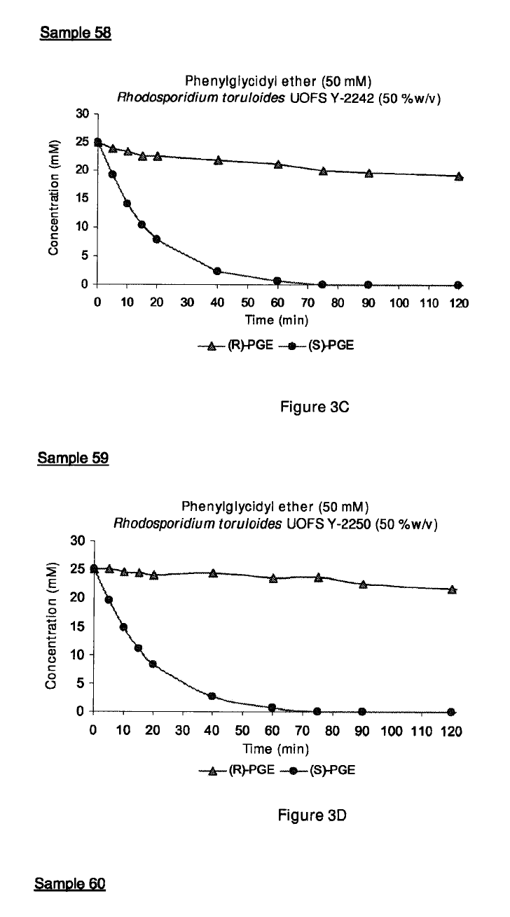

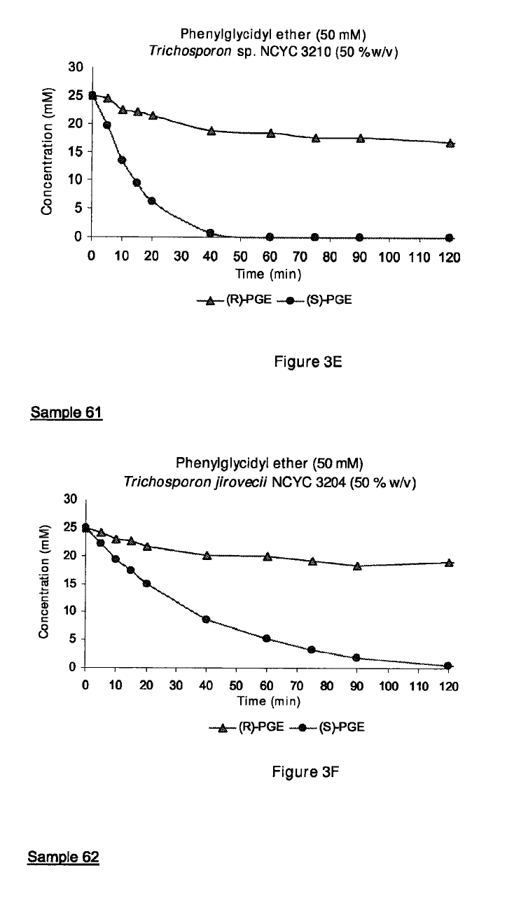

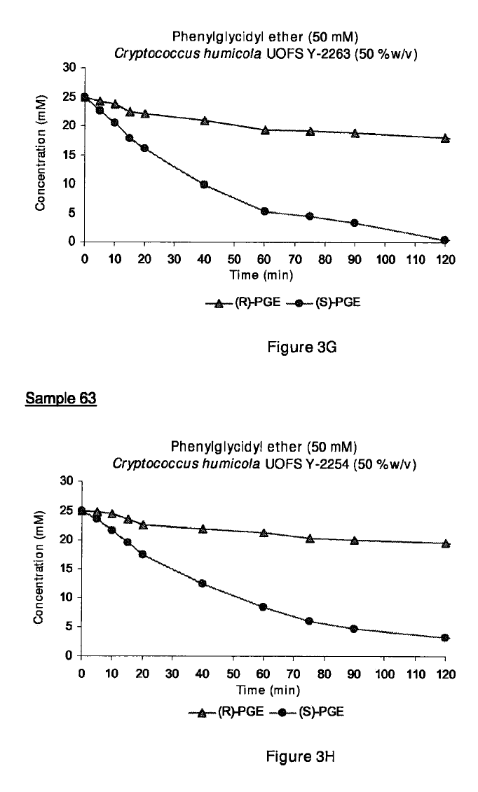

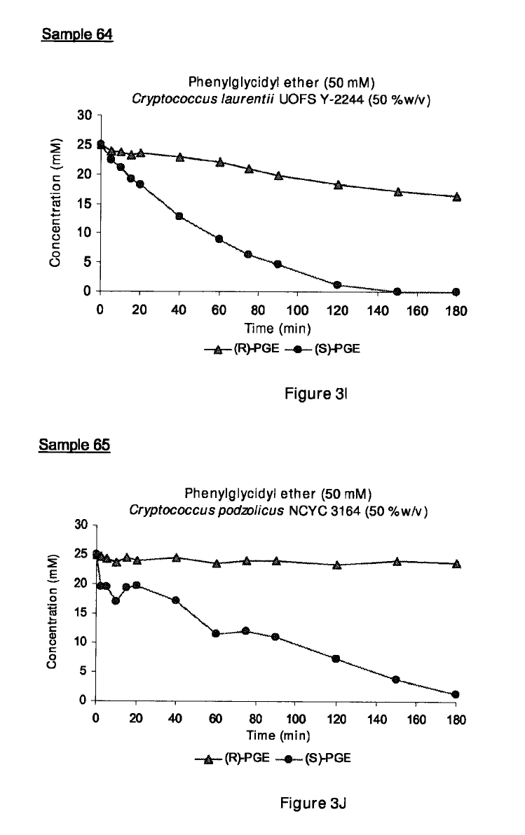

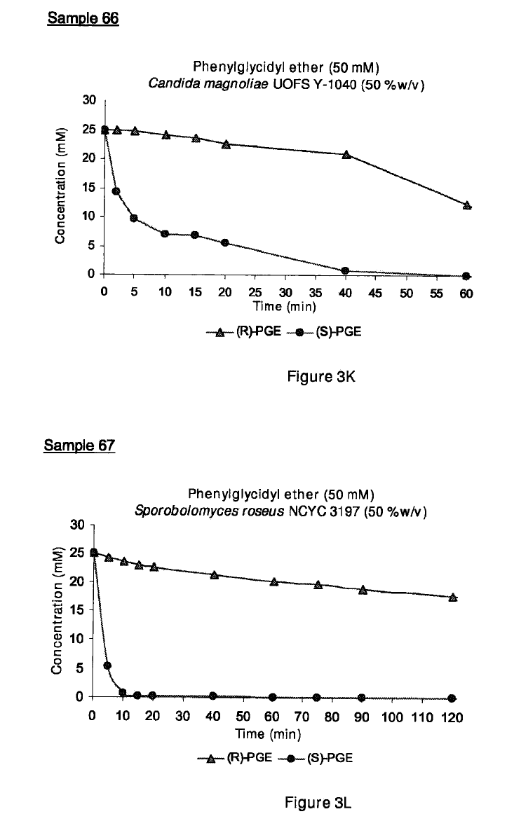

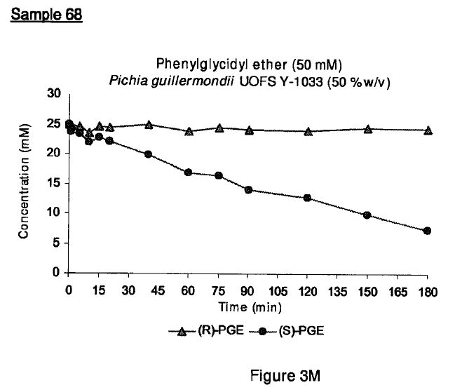

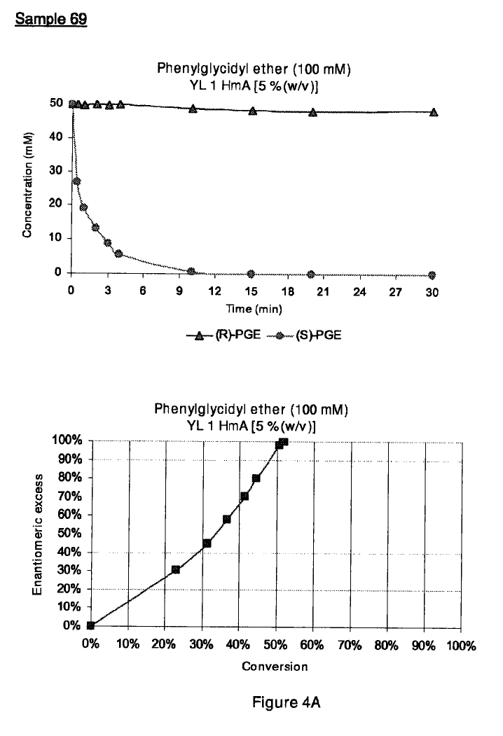

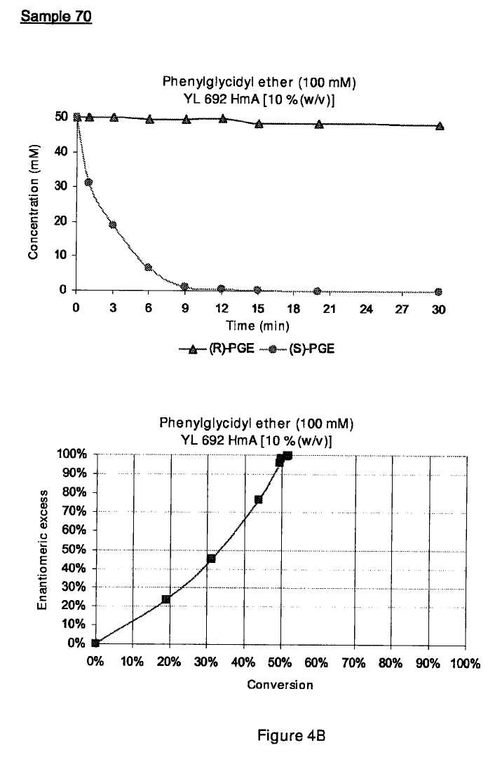

Samples 56-68 (FIGS. 3A-3M) illustrate the use of different wild type yeast strains selected from Table 2 to produce optically active phenyl glycidyl ethers and vicinal diols from racemic phenyl glycidyl ethers. The graphs show the change in concentrations of the glycidyl ether enantiomers with time.

Hydrolysis of (±)-phenyl glycidyl ether by yeasts selected from Table 2 was performed as described under general methods and materials at room temperature, unless otherwise stated. Biocatalyst concentrations (% m/v wet weight in the aqueous phase—equivalent to five-fold dry weight) are given on the graphs as are indications of the racemic substrate concentrations in millimolar.

### Example 4

### Hydrolysis of (±)-Phenyl Glycidyl Ether by Recombinant Yeast Expression Hosts Transformed with the Epoxide Hydrolase Genes from Selected Wild Type Yeast Strains to Produce Optically Active (R)-Phenyl Glycidyl Ether and the Corresponding (S)-Diol

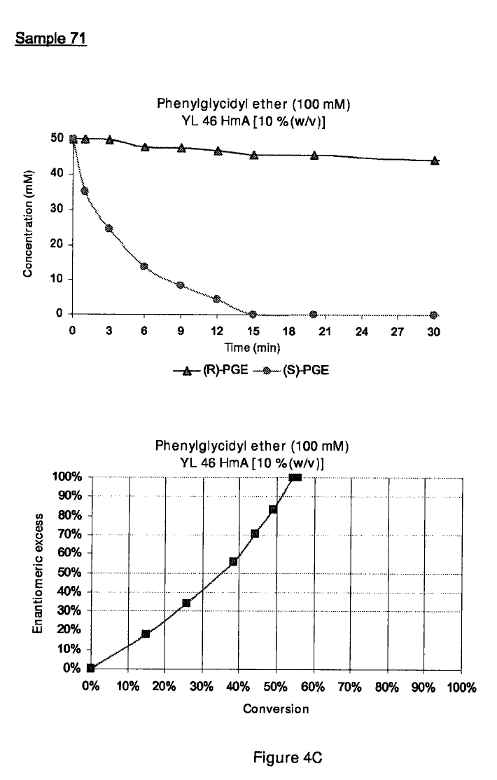

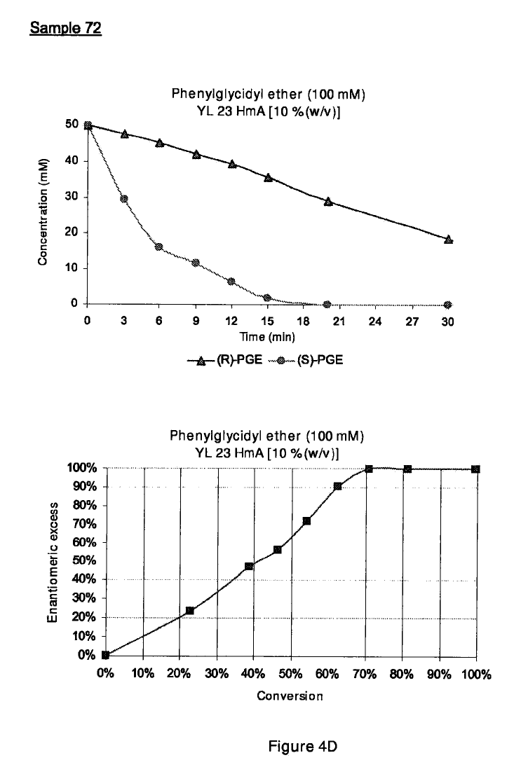

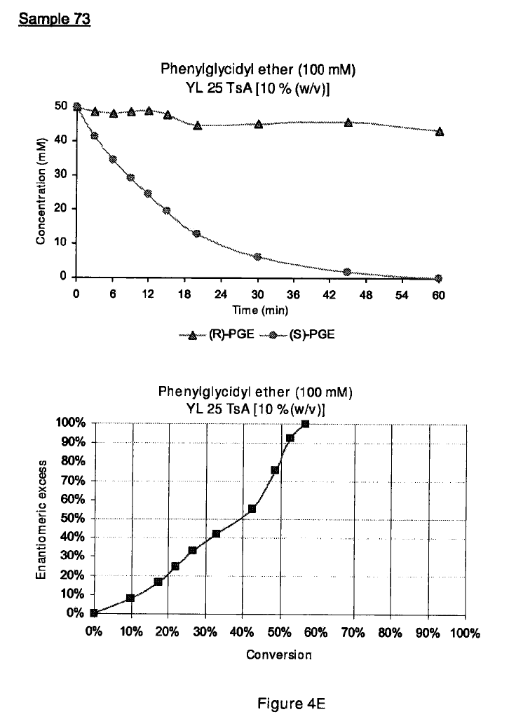

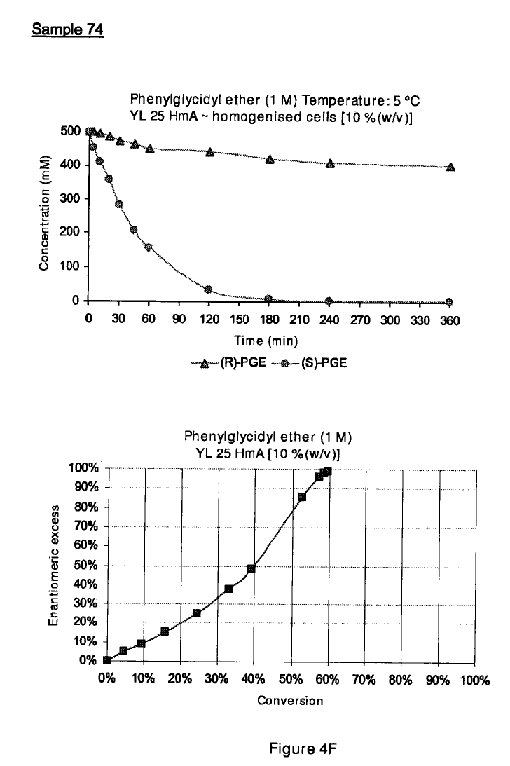

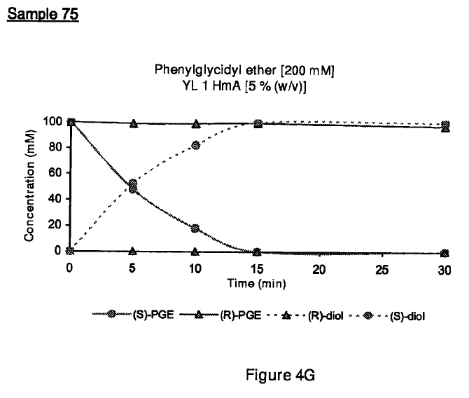

Samples 69-75 (FIGS. 4A-4G) illustrate the use of several recombinant yeast strains which overexpress in Yarrowia lipolytica several epoxide hydrolase genes selected from wild types defined in Table 2 to produce optically active phenyl glycidyl ethers and vicinal diols from racemic phenyl glycidyl ethers. The top graph in each figure shows the change in concentrations of the phenyl glycidyl ether enantiomers with time while the bottom graph in each figure shows the enantiomeric excess of the remaining epoxide at various conversions.

Hydrolysis of (±)-phenyl glycidyl ether by the recombinant strains was performed as described under general methods and materials at room temperature, unless otherwise stated. Biocatalyst concentrations (% m/v wet weight in the aqueous phase equivalent to five-fold dry weight) are given on the graphs as are indications of the racemic substrate concentrations in millimolar.

### Examples 5

### Selection of yeasts that are able to produce optically active (S)- or (R)-benzyl glycidyl ether (benzyloxypropylene oxide) and (S)- or (R)-3-benzyloxy-1,2-propanediol from (±)-benzyl glycidyl ether

Samples 76-176 in Table 3 illustrate examples of wild-type yeasts that were shown to be enantioselective on (±)-benzyl glycidyl ether with different enantioselectivities. Strains producing S-Benzyl glycidyl ether and S-diol have the “same” selectivity as displayed by yeasts for phenyl glycidyl ether i.e. that produce R-phenyl glycidyl ether, and is highlighted. The absolute configuration assignment changes because of a switch of priorities if the substitutents as defined by the Cahn-Ingold-Prelogg rule. Strains producing R-BGE and R-diol have the “opposite” selectivity as that displayed for phenyl glycidyl ether.

TABLE 3
Examples of yeast strains from different genera that hydrolyse benzyl glycidyl
ether enantioselectively^{a}

| | | | Culture | ees | Conv | Abs. |
|---|---|---|---|---|---|---|
| no. | Screen no | Species | collection no | (%) | (%) | conf |
| 76 | Jen 01 | Arxula adeninivorans | UOFS Y-1223 | −9.2 | 46.3 | S |
| 77 | 693 | Arxula terrestris | NCYC 3148 | −18.6 | 62.1 | S |
| 78 | 43* | Bullera dendrophila | NCYC 3152 [ | −20.0 | 26.3 | S |
| 79 | 69 | Candida famata | UOFS Y-0203 | −5.4 | 61.6 | S |
| 80 | 705 | Candida intermedia | UOFS Y-0964 | −7.0 | 60.3 | S |
| 81 | 677 | Candida magnoliae | UOFS Y-0799 | −20.4 | 62.1 | S |
| 82 | 678 | Candida magnoliae | UOFS Y-1297 | −15.7 | 6.5 | S |
| 83 | 751 | Candida magnoliae | UOFS Y-1040 | −29.1 | 71.4 | S |
| 84 | 708 | Candida rugosa | NCYC 3155 | −30.8 | 45.4 | S |
| 85 | POH 29 | Candida sp. (new) rel to C. sorbophila | NCYC 3217 | −3.2 | 45.7 | S |
| 86 | Jen 03 | Cryptococcus albidus | UOFS Y-0821 | 10.5 | 45.8 | R |
| 87 | Car 014 | Cryptococcus curvatus | UOFS Y-2225 | 4.7 | 39.7 | R |
| 88 | Car 054 | Cryptococcus curvatus | NCYC 3158 | 11.8 | 54.4 | R |
| 89 | Car 137 | Cryptococcus humicola | UOFS Y-2254 | 12.1 | 57.3 | R |
| 90 | Car 220(a) | Cryptococcus humicola | UOFS Y-2262 | 13.1 | 74.7 | R |
| 91 | Car 400 | Cryptococcus humicola | UOFS Y-2263 | 5.5 | 52.3 | R |
| 92 | TT 05 | Cryptococcus humicola | UOFS Y-0571 | 4.6 | 52.7 | R |
| 93 | Jen 15 | Cryptococcus hungaricus | NCYC 3159 | 10.5 | 28.3 | R |
| 94 | Car 099 | Cryptococcus laurentii | UOFS Y-2244 | 18.0 | 58.8 | R |
| 95 | AB 34 | Cryptococcus podzolicus | UOFS Y-1890 | 3.0 | 42.4 | R |
| 96 | AB 37 | Cryptococcus podzolicus | UOFS Y-1896 | 3.8 | 31.9 | R |
| 97 | AB 39 | Cryptococcus podzolicus | UOFS Y-1912 | 3.2 | 31.3 | R |
| 98 | AB 40 | Cryptococcus podzolicus | UOFS Y-1881 | 2.7 | 38.6 | R |
| 99 | AB 46 | Cryptococcus podzolicus | UOFS Y-1907 | 3.7 | 30.1 | R |
| 100 | AB 47 | Cryptococcus podzolicus | UOFS Y-1908 | 2.4 | 51.1 | R |
| 101 | AB 55 | Cryptococcus podzolicus | UOFS Y-1911 | 2.9 | 39.7 | R |
| 102 | AB 57 | Cryptococcus podzolicus | UOFS Y-1914 | 3.9 | 42.1 | R |
| 103 | AB 58 | Cryptococcus podzolicus | NCYC 3164 | −30.2 | 65.2 | S |
| 104 | Jen 22 | Cryptococcus terreus | NCYC 3166 | 3.4 | 37.5 | R |
| 105 | 17 | Debaryomyces hansenii | UOFS Y-0492 | −4.9 | −1.5 | S |
| 106 | 101 | Debaryomyces hansenii | UOFS Y-0604 | −3.3 | 22.9 | S |
| 107 | 104 | Debaryomyces hansenii | UOFS Y-0607 | −2.2 | 32 | S |
| 108 | 105 | Debaryomyces hansenii | UOFS Y-0608 | −4.3 | 21 | S |
| 109 | 111 | Debaryomyces hansenii | UOFS Y-0615 | −6.3 | 42.3 | S |
| 110 | 113 | Debaryomyces hansenii | NCYC 3167 | −3.0 | 46 | S |
| 111 | 45 B | Lipomyces sp. | UOFS Y-2159 B | −3.0 | 49.7 | S |
| 112 | 47 | Pichia guillermondii | UOFS Y-1028 | −3.8 | 53.9 | S |
| 113 | 112 | Pichia guillermondii | UOFS Y-0053 | −5.4 | 59.5 | S |
| 114 | 674 | Pichia guillermondii | UOFS Y-1030 | −5.2 | 43.1 | S |
| 115 | 675 | Pichia guillermondii | UOFS Y-1033 | −9.4 | 68.9 | S |
| 116 | 679 | Pichia guillermondii | UOFS Y-0054 | −8.4 | 69.9 | S |
| 117 | 702 | Pichia guillermondii | NCYC 3175 | −5.6 | 56.1 | S |
| 118 | 707 | Pichia guillermondii | NCYC 3174 | −2.6 | 45.9 | S |
| 119 | 28 | Pichia haplophila | UOFS Y-2136 | −17.3 | 67.2 | S |
| 120 | 676 | Pichia haplophila | NCYC 3176 | −19.5 | 63.8 | S |
| 121 | 169 | Rhodosporidium lusitaniae | NCYC 3178 | 8.0 | 28.1 | R |
| 122 | 692 | Rhodosporidium paludigenum | NCYC 3179 | 4.0 | 24.6 | R |
| 123 | 48 | Rhodosporidium paludigenum | UOFS Y-0481 | 1.7 | 25.9 | R |
| 124 | 671 | Rhodosporidium toruloides | UOFS Y-0472 | 4.5 | 28.7 | R |
| 125 | Car 003 | Rhodosporidium toruloides | UOFS Y-2222 | 3.3 | 39.1 | R |
| 126 | Car 006 | Rhodosporidium toruloides | UOFS Y-2223 | 7.7 | 41.7 | R |
| 127 | Car 020 | Rhodosporidium toruloides | UOFS Y-2226 | 10.4 | 43.7 | R |
| 128 | Car 052 | Rhodosporidium toruloides | UOFS Y-2230 | 33.2 | 60.3 | R |
| 129 | Car 059 | Rhodosporidium toruloides | UOFS Y-2231 | 8.8 | 47.7 | R |
| 130 | Car 067 | Rhodosporidium toruloides | UOFS Y-2236 | 3.0 | 29.3 | R |
| 131 | Car 070 | Rhodosporidium toruloides | UOFS Y-2237 | 4.6 | 30.6 | R |
| 132 | Car 076 | Rhodosporidium toruloides | UOFS Y-2238 | 11.1 | 45.6 | R |
| 133 | Car 077 | Rhodosporidium toruloides | UOFS Y-2239 | 5.1 | 36.5 | R |
| 134 | Car 078 | Rhodosporidium toruloides | UOFS Y-2240 | 10.7 | 37.4 | R |
| 135 | Car 092 | Rhodosporidium toruloides | UOFS Y-2241 | 8.7 | 42.8 | R |
| 136 | Car 093 | Rhodosporidium toruloides | UOFS Y-2242 | 7.5 | 30.9 | R |
| 137 | Car 094 | Rhodosporidium toruloides | UOFS Y-2243 | 15.4 | 54.2 | R |
| 138 | Car 100 | Rhodosporidium toruloides | UOFS Y-2245 | −8.8 | 55.3 | S |
| 139 | Car 103 | Rhodosporidium toruloides | UOFS Y-2246 | 3.4 | 29.8 | R |
| 140 | Car 118 | Rhodosporidium toruloides | NCYC 3182 | 6.2 | 39.8 | R |
| 141 | Car 120 | Rhodosporidium toruloides | UOFS Y-2249 | 7.2 | 43.8 | R |
| 142 | Car 121 | Rhodosporidium toruloides | UOFS Y-2250 | 3.0 | 35.8 | R |
| 143 | Car 126 | Rhodosporidium toruloides | UOFS Y-2251 | 4.6 | 33.7 | R |
| 144 | Car 134 | Rhodosporidium toruloides | UOFS Y-2253 | 3.7 | 43 | R |
| 145 | Car 142 | Rhodosporidium toruloides | UOFS Y-2255 | 4.8 | 30.6 | R |
| 146 | Car 200 | Rhodosporidium toruloides | UOFS Y-2256 | 14.1 | 35.3 | R |
| 147 | Car 204 | Rhodosporidium toruloides | UOFS Y-2257 | 5.3 | 40 | R |
| 148 | Car 205A | Rhodosporidium toruloides | UOFS Y-2258 | 12.0 | 46.9 | R |
| 149 | Car 209 | Rhodosporidium toruloides | UOFS Y-2260 | 4.3 | 33.6 | R |
| 150 | Car 210 | Rhodosporidium toruloides | UOFS Y-2261 | 3.1 | 19.3 | R |
| 151 | POH 20 | Rhodosporidium toruloides | NCYC 3216 | 2.2 | 41.1 | R |
| 152 | POH 28 | Rhodosporidium toruloides | NCYC 3215 | 5.8 | 41.9 | R |
| 153 | 25 | Rhodotorula araucariae | NCYC 3183 | 7.0 | 51 | R |
| 154 | EP 230 | Rhodotorula aurantiaca | NCYC 3185 | 4.9 | 45.2 | R |
| 155 | 681 | Rhodotorula glutinis | UOFS Y-0653 | 13.7 | 38.6 | R |
| 156 | 713 | Rhodotorula glutinis | UOFS Y-0489 | 10.0 | 52.5 | R |
| 157 | Car 022 | Rhodotorula glutinis | UOFS Y-2227 | 9.0 | 41.3 | R |
| 158 | Car 060 | Rhodotorula glutinis | UOFS Y-2232 | 3.5 | 55.2 | R |
| 159 | Car 061 | Rhodotorula glutinis | UOFS Y-2233 | 8.5 | 50.7 | R |
| 160 | Car 062 | Rhodotorula glutinis | UOFS Y-2234 | 10.0 | 65.3 | R |
| 161 | 714 | Rhodotorula minuta | NCYC 3187 | 9.6 | 53.6 | R |
| 162 | 682 | Rhodotorula mudilaginosa | UOFS Y-0478 | 5.3 | 23.6 | R |
| 163 | 690 | Rhodotorula sp. nearest minuta | UOFS Y-0125 | 2.3 | 40.4 | R |
| 164 | 697 | Rhodotorula sp. Minuta/mucilaginosa | UOFS Y-0958 | 6.4 | 40.6 | R |
| 165 | 698 | Rhodotorula sp. Minuta/mucilaginosa | UOFS Y-0959 | 5.6 | 48 | R |
| 166 | 174 | Rhodotorula philyla | NCYC 3191 | 9.7 | 30.7 | R |
| 167 | 24 | Rhodotorula sp. | UOFS Y-2042 | 3.4 | 44.2 | R |
| 168 | 37 | Rhodotorula sp. | UOFS Y-0448 | 12.4 | 48.9 | R |
| 169 | 165 | Rhodotorula sp. | NCYC 3193 | 3.7 | 32 | R |
| 170 | Jen 31 | Sporidiobolus salmonicolor | NCYC 3196 | 5.9 | 45.3 | R |
| 171 | Jen 30 | Sporobolomyces holsaticus | NCYC 3198 | 5.9 | 37.2 | R |
| 172 | 285 | Sporobolomyces roseus | NCYC 3197 | 7.0 | 44 | R |
| 173 | 22 | Trichosporon jirovecii | NCYC 3204 | 18.3 | 59 | R |
| 174 | 14 | Trichosporon mucoides | NCYC 3205 | 13.3 | 49.3 | R |
| 175 | 231 | Trichosporon sp. | NCYC 3210 | 15.1 | 42.5 | R |
| 176 | TT 33 | Yarrowia lipolytica | UOFS Y-0647 | 6.2 | 50.4 | R |

^{a}Reaction conditions: 50 mM benzyl glycidyl ether, 50% cells (w/v) in phosphate buffer (50 mM, pH 7.5), Reaction time 3 hours.

### Example 6

### Hydrolysis of (±)-benzyl glycidyl ether by selected wild type yeasts to produce optically active benzyl glycidyl ether and the corresponding optically active 3-benzyloxy-1,2-propanediol

These samples illustrate the use of different wild type yeast strains selected from Table 3 to produce optically active glycidyl ethers and vicinal diols from glycidyl ethers. The graphs show the change in concentrations of the glycidyl ether enantiomers with time. Hydrolysis of (±)-benzyl glycidyl ether by yeasts selected from Table 3 was performed as described under general methods and materials at room temperature, unless otherwise stated. Substrate concentrations (mM) and biocatalyst concentrations (% m/v wet weight—equivalent to fivefold % m/v dry weight in the aqueous phase) are given on the graphs.

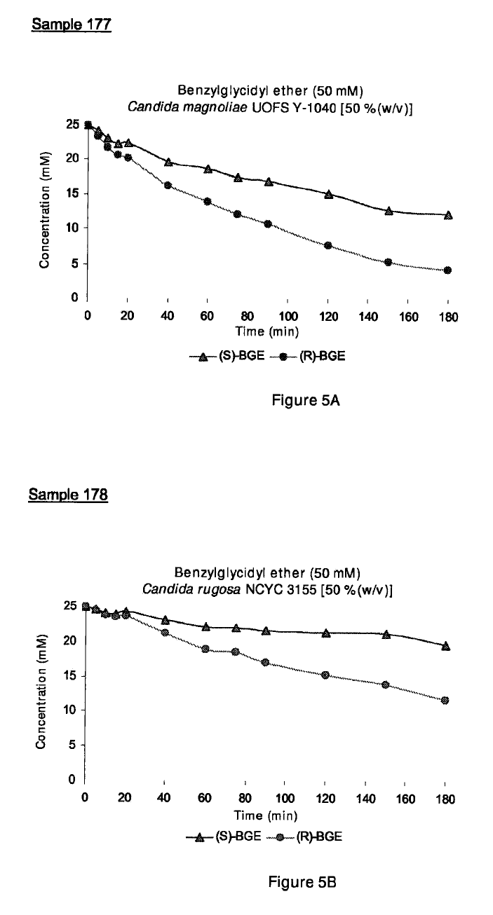

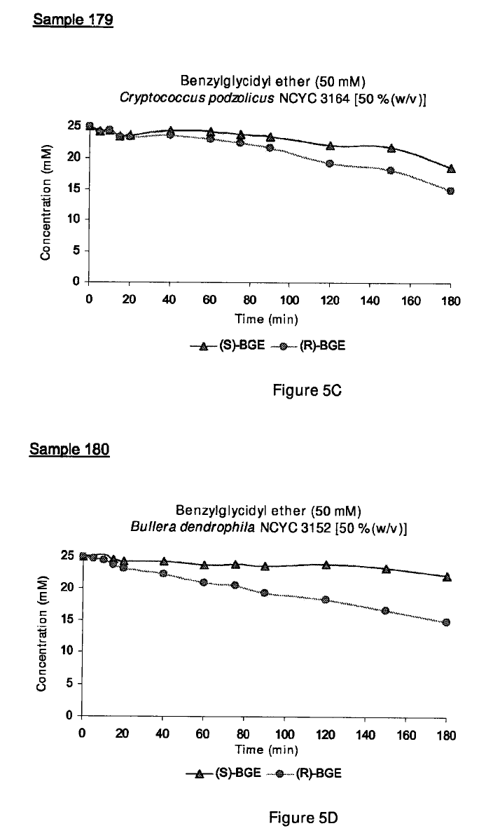

Samples 177-180 (FIGS. 5A-5D) graphically illustrate the chiral preference of the hydrolysis of (±)-benzyl glycidyl ether by selected wild type yeasts to produce optically active (S)-benzyl glycidyl ether and the corresponding (S)-diol.

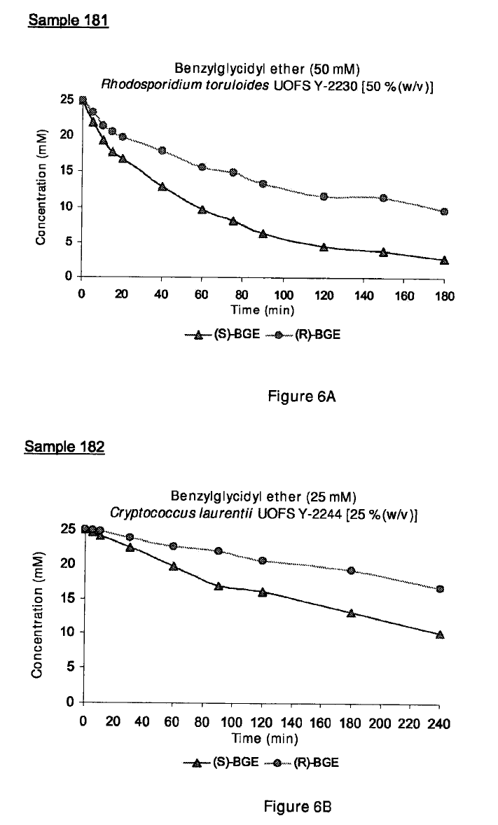

Samples 181 and 182 (FIGS. 6A and 6B) graphically illustrates the chiral preference of the hydrolysis of (±)-benzyl glycidyl ether by a selected wild type yeast to produce optically active (R)-benzyl glycidyl ether and the corresponding (R)-diol.

### Example 7

### Hydrolysis of (±)-benzyl glycidyl ether by recombinant yeast expression hosts transformed with the epoxide hydrolase genes from selected wild type yeast strains to produce to produce optically active benzyl glycidyl ether and the corresponding optically active 3-benzyloxy-1,2-propanediol.

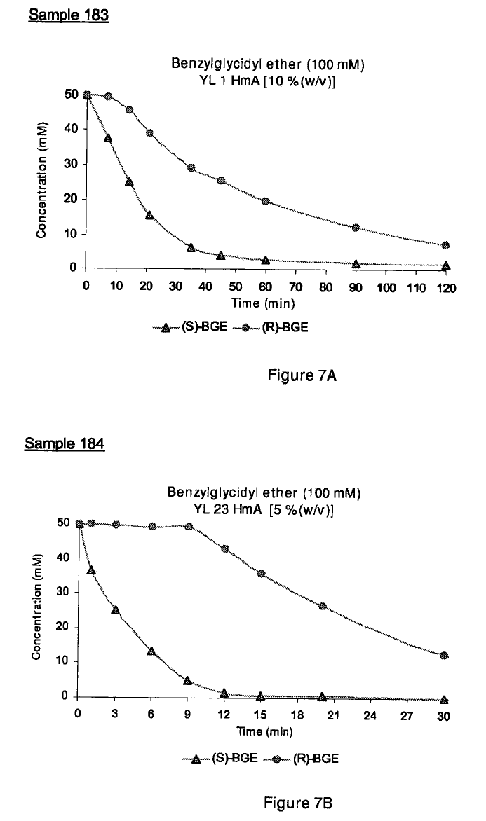

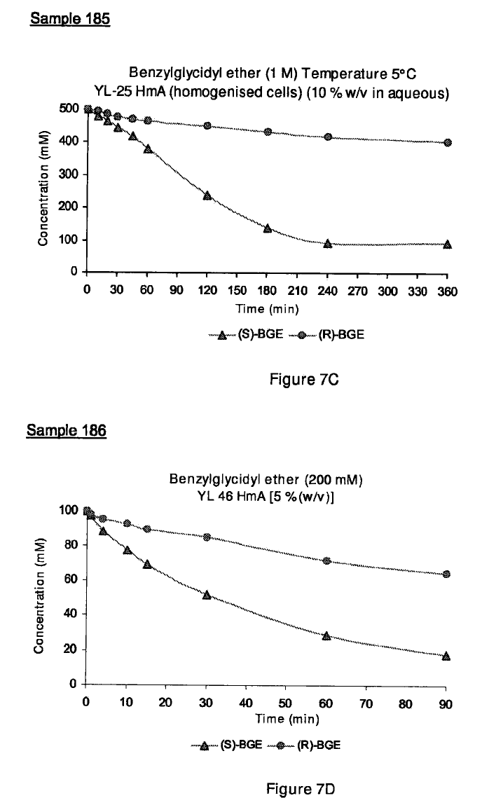

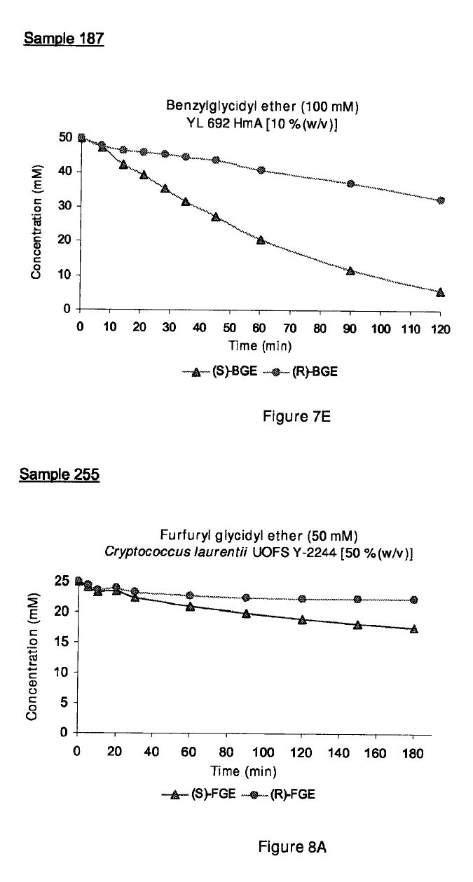

Samples 183-187 (FIGS. 7A-7E) graphically illustrates the hydrolysis of (±)-benzyl glycidyl ether by recombinant yeast expression hosts transformed with the epoxide hydrolase genes from selected wild type yeast strains to produce optically active (R)-benzyl glycidyl ether and the corresponding (R)-3-benzyloxy-1,2-propanediol. Substrate concentrations (mM) and biocatalyst concentrations (% m/v wet weight—equivalent to fivefold % m/v dry weight in the aqueous phase) are given on the graphs.

### Example 8

### Hydrolysis of (±)-furfuryl glycidyl ether by selected wild type yeasts to produce optically active (S)- or (R)-furfuryl glycidyl ether (furfuryloxypropylene oxide) and (R)- or (S)-3-furfuryloxy-1,2-propanediol

Samples 188-254 in Table 4 illustrate the stereoselective hydrolysis of furfuryl glycidyl ether (FGE) by selected wild-type yeasts.

TABLE 4
Examples of yeast strains from different genera that hydrolyse furfuryl glycidyl
ether enantioselectively^{a}. Positive ee values denote yeast that preferentially hydrolyse
(R)-FGE to produce optically active (S)-FGE and (S)-diol (highlighted), while negative
ee values denote yeast that preferentially hydrolyse (S)-FGE to produce optically active
(R)-FGE and (R)-diol.

| | | | | (S)- | (R)- | | | |
|---|---|---|---|---|---|---|---|---|
| | Screen | | Culture | FGE | FGE | eeₛ | Conv | Abs. |
| Nr. | no. | Species | collection nr. | (mM) | (mM) | (%) | (%) | conf. |
| 188 | Jen 08 | Arxula adeninivorans | UOFS Y-1222 | 4.34 | 3.72 | 7.7 | 35.5 | S |
| 189 | Jen 01 | Arxula adeninivorans | UOFS Y-1223 | 5.25 | 4.42 | 8.5 | 22.6 | S |
| 190 | Jen25 | Bullera dendrophila | NCYC 3152 | 5.74 | 6.50 | −6.2 | 2.1 | R |
| 191 | Jen26 | Bullera dendrophila | NCYC 3208 | 5.24 | 6.25 | −8.9 | 8.1 | R |
| 192 | 705 | Candida intermedia | UOFS Y-0964 | 1.42 | 1.27 | 5.4 | 78.5 | S |
| 193 | 677 | Candida magnoliae | UOFS Y-0799 | 3.00 | 2.22 | 14.9 | 58.2 | S |
| 194 | Jen03 | Cryptococcus albidus | UOFS Y-0821 | 0.31 | 0.36 | −7.5 | 94.6 | R |
| 195 | Car054 | Cryptococcus curvatus | NCYC 3158 | 0.11 | 0.23 | −34.2 | 97.2 | R |
| 196 | Car220 | Cryptococcus humicola | UOFS Y-2262 | 5.42 | 5.93 | −4.5 | 9.2 | R |
| 197 | Car400 | Cryptococcus humicola | UOFS Y-2263 | 2.02 | 2.23 | −4.9 | 66.0 | R |
| 198 | Jen15 | Cryptococcus hungaricus | NCYC 3159 | 0.50 | 0.69 | −15.6 | 90.5 | R |
| 199 | Car099 | Cryptococcus laurentii | UOFS Y-2244 | 3.00 | 4.05 | −14.8 | 43.6 | R |
| 200 | Jen14 | Cryptococcus macerans | NCYC 3163 | 0.58 | 0.67 | −7.3 | 90.0 | R |
| 201 | AB43 | Cryptococcus podzolicus | UOFS Y-1902 | 2.16 | 2.01 | 3.4 | 66.7 | S |
| 202 | AB46 | Cryptococcus podzolicus | UOFS Y-1907 | 2.39 | 2.82 | −8.4 | 58.3 | R |
| 203 | AB49 | Cryptococcus podzolicus | UOFS Y-1882 | 0.87 | 0.69 | 11.6 | 87.5 | S |
| 204 | AB58 | Cryptococcus podzolicus | NCYC 3164 | 0.53 | 0.47 | 6.0 | 92.0 | S |
| 205 | AB40 | Cryptococcus podzolicus | UOFS Y-1881 | 0.49 | 0.67 | −15.8 | 90.7 | R |
| 206 | 109 | Debaryomyces hansenii | UOFS Y-0613 | 1.77 | 1.40 | 11.6 | 74.6 | S |
| 207 | 113 | Debaryomyces hansenii | NCYC 3167 | 5.95 | 5.70 | 2.1 | 6.8 | S |
| 208 | 173 | Exophiala dermatitidis | NCYC 3227 | 4.55 | 3.81 | 8.9 | 33.2 | S |
| 209 | 45b | Lipomyces sp. (course) | UOFS Y-2159 B | 5.26 | 4.86 | 3.9 | 19.1 | S |
| 210 | 45a | Lipomyces sp. (smooth) | UOFS Y-2159 A | 4.96 | 4.70 | 2.7 | 22.7 | S |
| 211 | 674 | Mastigomyces philipporii | UOFS Y-1139 | 4.33 | 4.01 | 3.7 | 33.3 | S |
| 212 | 41 | Myxozyma melibiosi | NCYC 3172 | 6.67 | 7.14 | −3.4 | 10.5 | R |
| 213 | 112 | Pichia guillermondii | UOFS Y-0053 | 6.13 | 5.26 | 7.7 | 8.9 | S |
| 214 | 675 | Pichia guillermondii | UOFS Y-1033 | 3.33 | 3.15 | 2.7 | 48.2 | S |
| 215 | 47 | Pichia guillermondii | UOFS Y-1028 | 1.64 | 1.48 | 5.4 | 75.1 | S |
| 216 | 707 | Pichia guillermondii | NCYC 3174 | 0.40 | 0.32 | 10.6 | 94.2 | S |
| 217 | 706 | Pichia guillermondii | UOFS Y-0057 | 0.45 | 0.37 | 10.1 | 93.5 | S |
| 218 | 676 | Pichia haplophila | NCYC 3176 | 0.67 | 0.77 | −7.0 | 88.5 | R |
| 219 | 28 | Pichia haplophila | UOFS Y-2136 | 0.20 | 0.16 | 9.7 | 97.1 | S |
| 220 | car77 | Rhodosporidium toruloides | UOFS Y-2239 | 5.95 | 6.35 | −3.2 | 1.6 | R |
| 221 | car76 | Rhodosporidium toruloides | UOFS Y-2238 | 3.79 | 4.12 | −4.2 | 36.7 | R |
| 222 | car52 | Rhodosporidium toruloides | UOFS Y-2230 | 4.61 | 6.05 | −13.5 | 14.7 | R |
| 223 | car20 | Rhodosporidium toruloides | UOFS Y-2226 | 4.96 | 5.51 | −5.3 | 16.3 | R |
| 224 | AB 1 | Rhodosporidium toruloides | NCYC 3181 | 4.89 | 5.64 | −7.2 | 15.7 | R |
| 225 | car78 | Rhodosporidium toruloides | UOFS Y-2240 | 5.15 | 5.44 | −2.7 | 15.2 | R |
| 226 | 46 | Rhodosporidium toruloides | UOFS Y-0471 | 4.67 | 5.45 | −7.7 | 19.0 | R |
| 227 | car6 | Rhodosporidium toruloides | UOFS Y-2223 | 3.69 | 5.94 | −23.4 | 23.0 | R |
| 228 | car205a | Rhodosporidium toruloides | UOFS Y-2258 | 3.31 | 3.66 | −5.0 | 44.2 | R |
| 229 | car100 | Rhodosporidium toruloides | UOFS Y-2245 | 2.76 | 3.26 | −8.3 | 51.9 | R |
| 230 | car200 | Rhodosporidium toruloides | UOFS Y-2256 | 2.86 | 3.19 | −5.4 | 51.6 | R |
| 231 | car121 | Rhodosporidium toruloides | UOFS Y-2250 | 2.67 | 3.44 | −12.6 | 51.1 | R |
| 232 | car108 | Rhodosporidium toruloides | UOFS Y-2247 | 2.24 | 2.41 | −3.8 | 62.8 | R |
| 233 | car3 | Rhodosporidium toruloides | UOFS Y-2222 | 0.51 | 0.93 | −29.3 | 88.5 | R |
| 234 | 2 | Rhodosporidium toruloides | UOFS Y-0518 | 0.55 | 1.47 | −45.2 | 83.8 | R |
| 235 | 25 | Rhodotorula araucariae | NCYC 3183 | 2.68 | 4.86 | −28.8 | 39.7 | R |
| 236 | 6 | Rhodotorula glutinis | UOFS Y-0513 | 1.16 | 2.88 | −42.6 | 67.6 | R |
| 237 | 50 | Rhodotorula glutinis | NCYC 3186 | 1.22 | 1.76 | −18.1 | 76.2 | R |
| 238 | 681 | Rhodotorula glutinis | UOFS Y-0653 | 0.52 | 0.84 | −23.5 | 89.1 | R |
| 239 | car62 | Rhodotorula glutinis | UOFS Y-2234 | 1.17 | 1.78 | −20.6 | 76.4 | R |
| 240 | 24 | Rhodotorula sp. | UOFS Y-2042 | 4.09 | 5.20 | −11.9 | 25.7 | R |
| 241 | 37 | Rhodotorula sp. | UOFS Y-0448 | 3.45 | 4.38 | −11.8 | 37.3 | R |
| 242 | jen31 | Sporidiobolus salmonicolor | NCYC 3196 | 0.77 | 0.84 | −4.4 | 87.2 | R |
| 243 | jen30 | Sporobolomyces holsaticus | NCYC 3198 | 0.44 | 0.49 | −5.1 | 92.5 | R |
| 244 | 228 | Trichosporon beigelii | UOFS Y-1580 | 6.00 | 5.30 | 6.2 | 9.7 | S |
| 245 | 232 | Trichosporon cutaneum var. | NCYC 3202 | 4.56 | 6.07 | −14.3 | 15.0 | R |
| | | cutaneum | | | | | | |
| 246 | 22 | Trichosporon jirovecii | NCYC 3204 | 4.14 | 4.59 | −5.1 | 30.2 | R |
| 247 | bv04 | Trichosporon moniliiforme | NCYC 3214 | 4.05 | 4.52 | −5.4 | 31.4 | R |
| 248 | 14 | Trichosporon mucoides | NCYC 3205 | 2.60 | 2.80 | −3.6 | 56.8 | R |
| 249 | 15 | Trichosporon mucoides | NCYC 3206 | 4.07 | 4.49 | −4.9 | 31.5 | R |
| 250 | 223 | Trichosporon mucoides | UOFS Y-0116 | 5.30 | 5.77 | −4.2 | 11.4 | R |
| 251 | 61 | Trichosporon pullulans | NCYC 3209 | 5.62 | 5.13 | 4.5 | 14.0 | S |
| 252 | 59 | Trichosporon sp. | UOFS Y-0861 | 3.36 | 4.29 | −12.1 | 38.8 | R |
| 253 | 152 | Trichosporon sp. | UOFS Y-0451 | 4.68 | 4.93 | −2.6 | 23.1 | R |
| 254 | 49 | Unidentified black yeast | UOFS Y-1938 | 5.14 | 6.08 | −8.4 | 10.3 | R |

^{a}Reaction conditions: 50 mM furfuryl glycidyl ether, 50% cells (w/v) in phosphate buffer (50 mM, pH 7.5), Reaction time 3 hours.

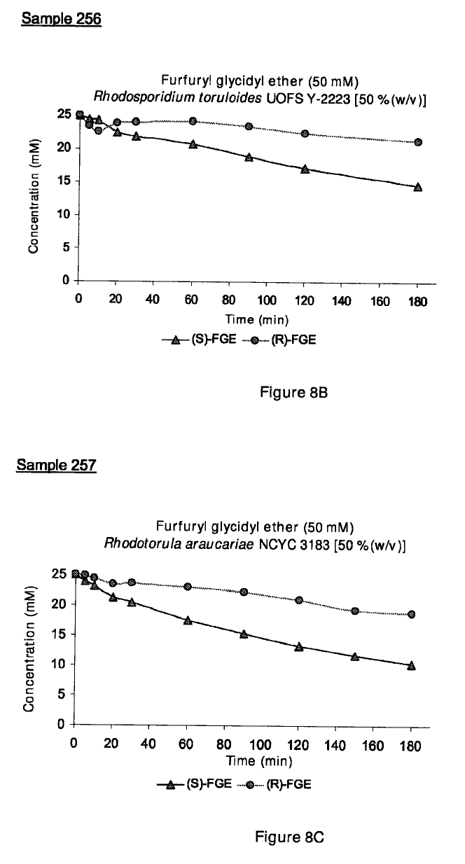

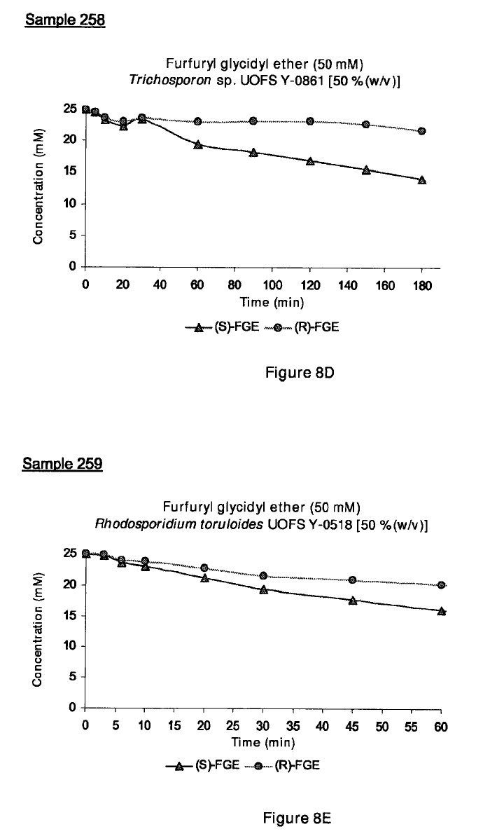

Samples 255-254 (FIGS. 8A-8D) graphically illustrate the hydrolysis of (±)-furfuryl glycidyl ether by wild type yeasts selected from Table 4 to produce optically active (R) furfuryl glycidyl ether and the corresponding optically active (R) 3-furfuryloxy-1,2-propanediol. The graphs show the change in concentrations of the glycidyl ether enantiomers with time. The hydrolysis of (±)-furfuryl glycidyl ether by the wild type yeasts was performed as described under general methods and materials at room temperature, unless otherwise stated. The substrate concentrations (mM) and the biocatalyst concentrations (% m/v wet weight biocatalyst loading in aqueous phase [equivalent to five-fold dry concentration]) are indicated in indicated in the graphs.

### Example 9

### Hydrolysis of (±)-furfuryl glycidyl ether by recombinant yeast expression hosts transformed with the epoxide hydrolase genes from selected wild type yeast strains to produce to produce optically active (S)- or (R)-furfuryl glycidyl ether (furfuryloxypropylene oxide) and (R)- or (S)-3-furfuryloxy-1,2-propanediol

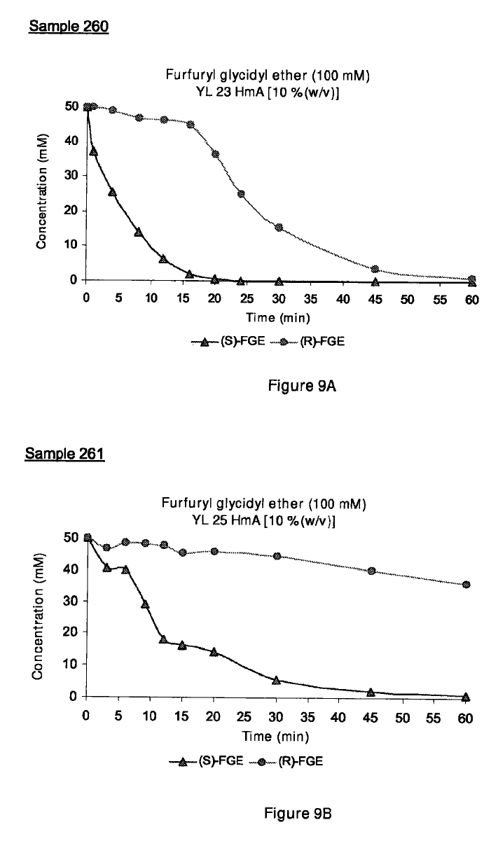

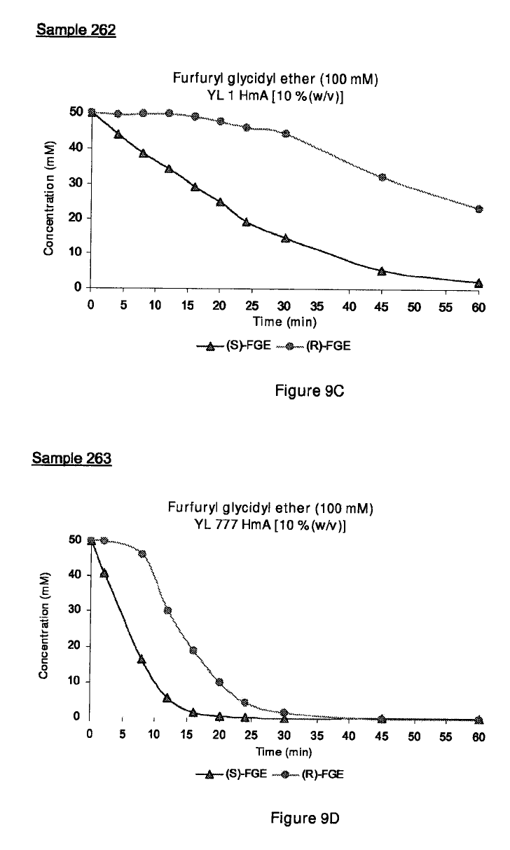

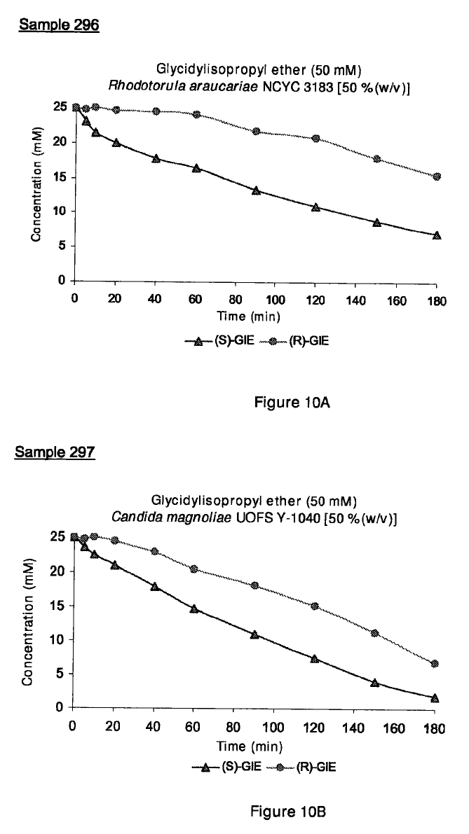

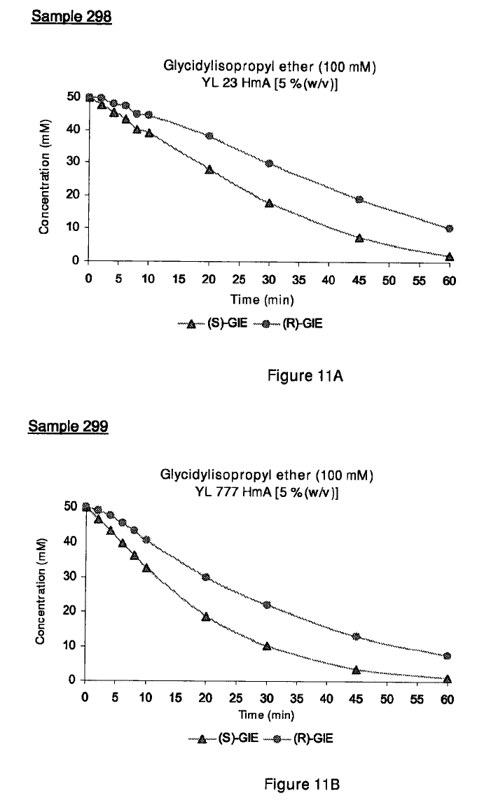

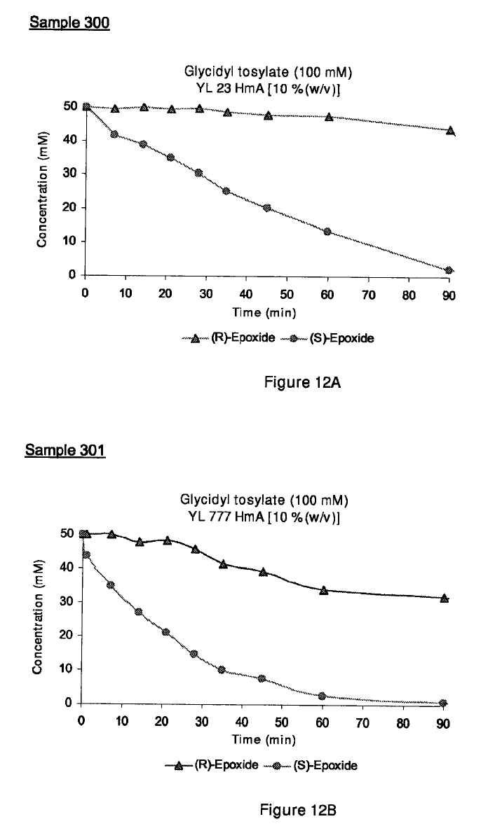

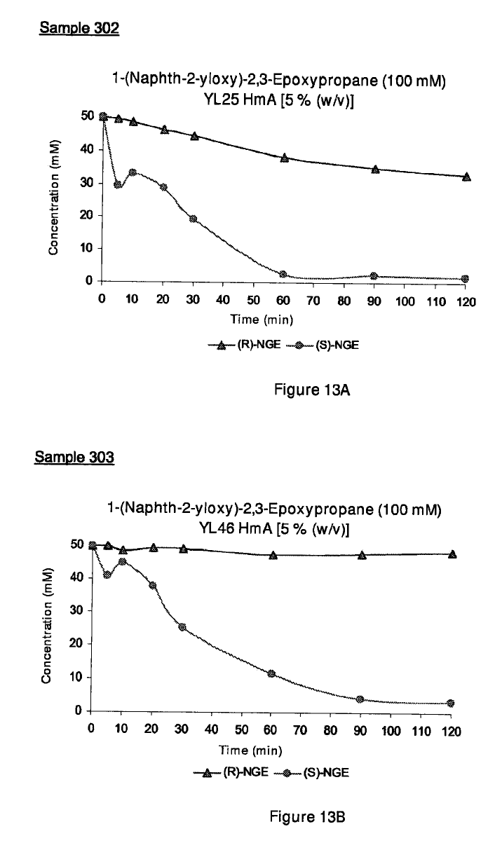

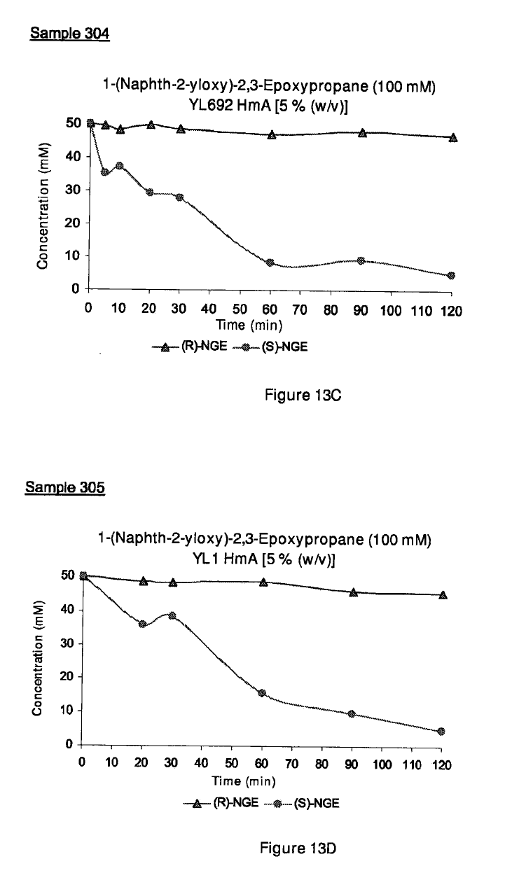

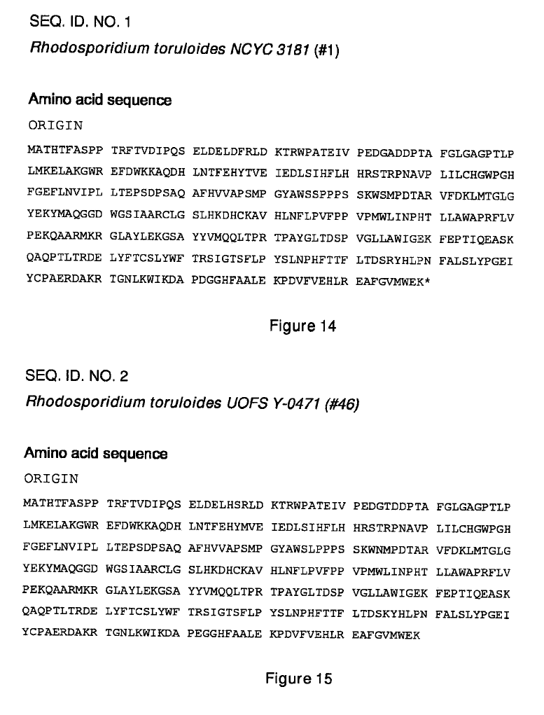

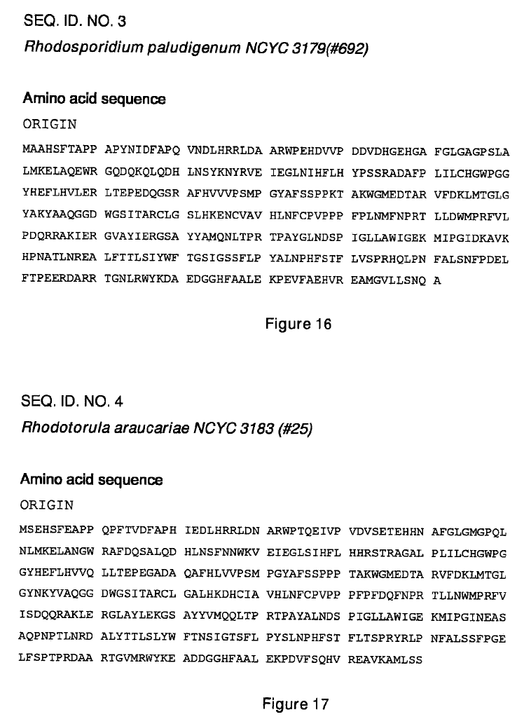

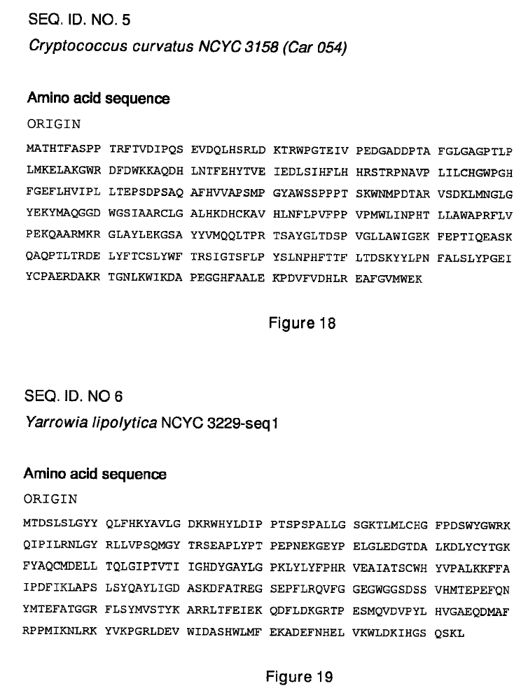

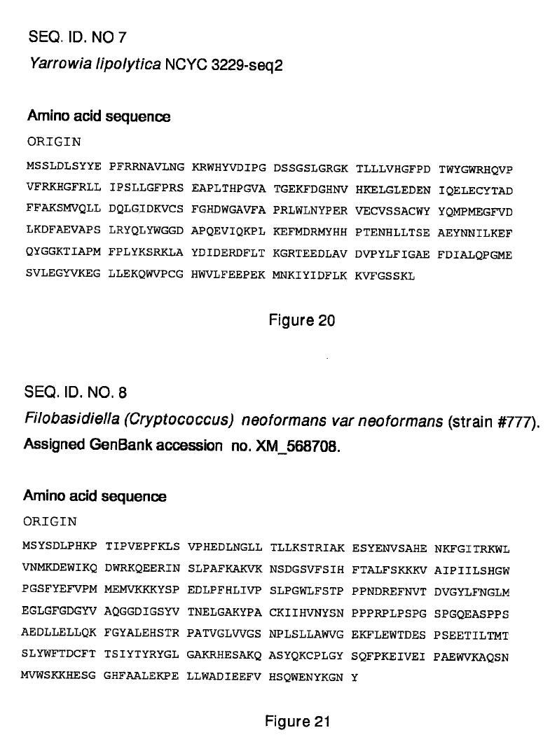

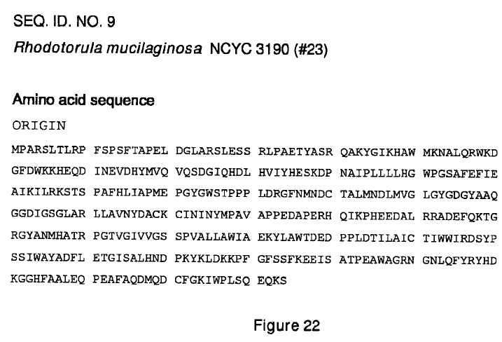

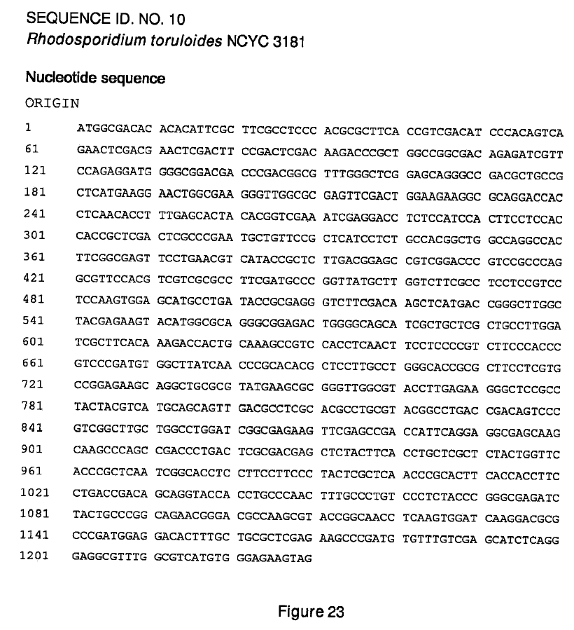

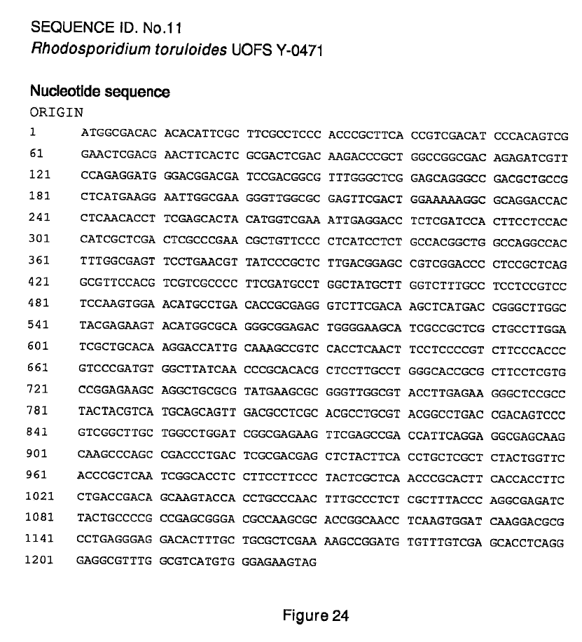

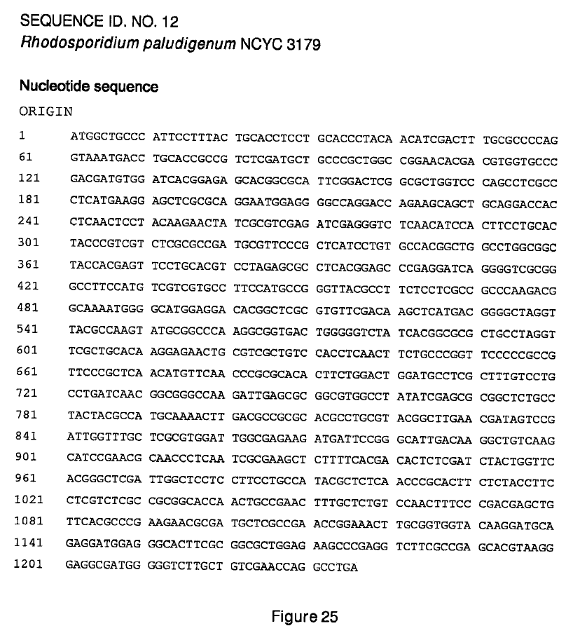

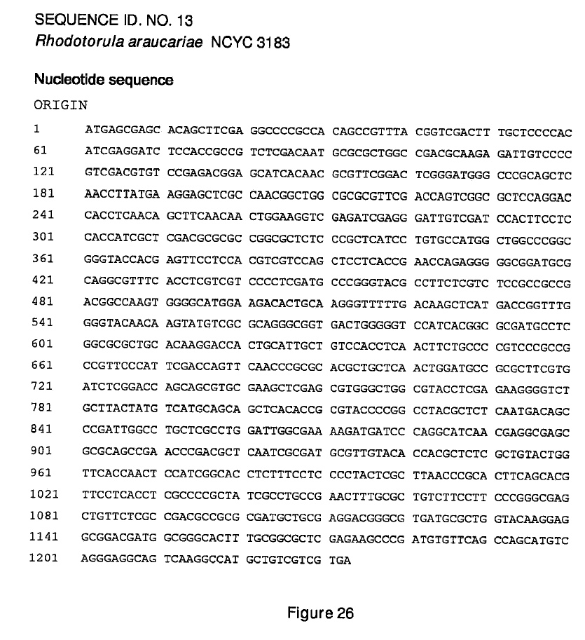

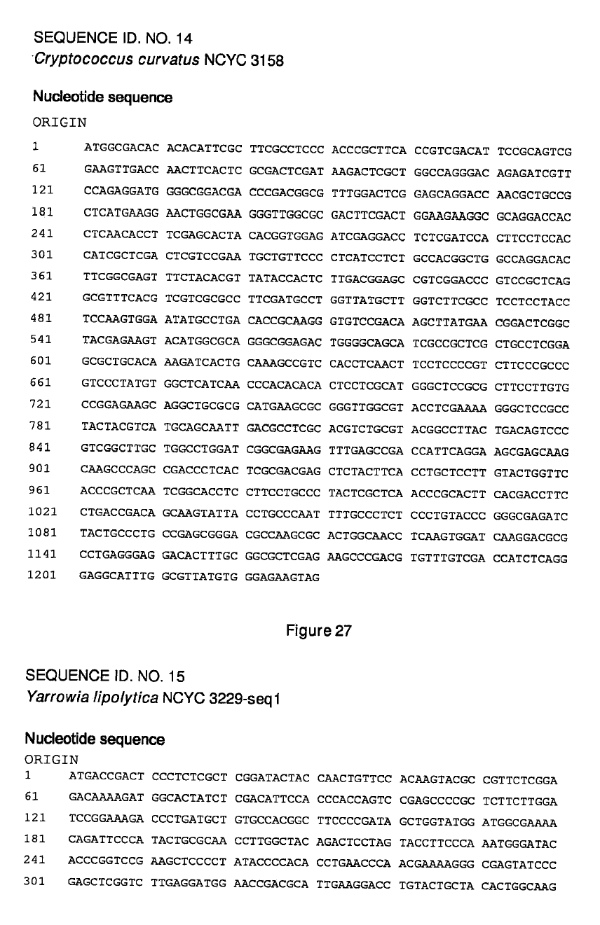

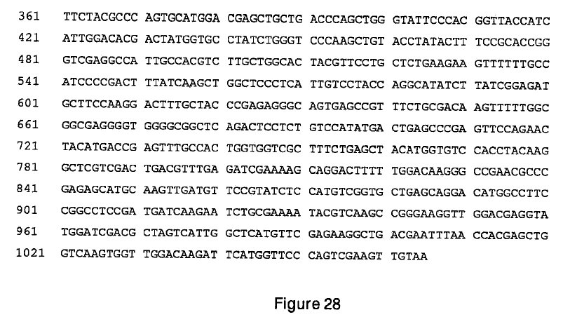

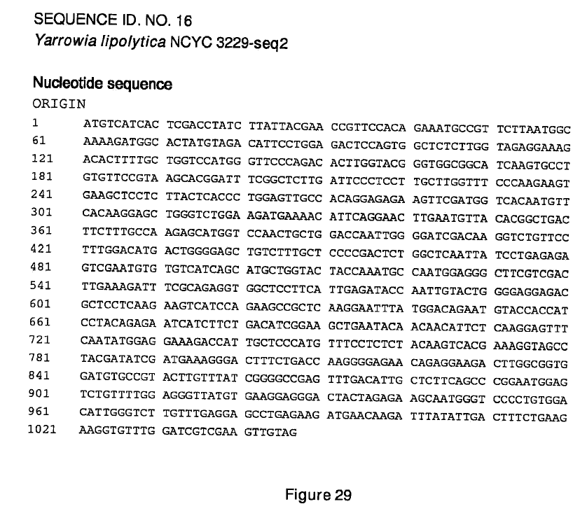

Samples 260-258 (FIGS. 9A-9D) graphically illustrate the hydrolysis of (±)-furfuryl glycidyl ether by recombinant yeast expression hosts transformed with the epoxide hydrolase genes from selected wild type yeast strains to produce optically active (R)-furfuryl glycidyl ether and the corresponding (R)-3-furfuryloxy-1,2-propanediol.

### Example 10

### Hydrolysis of (±)-Isopropyl glycidyl ether (1,2-epoxy-3-isopropoxypropane) by selected wild type and recombinant yeasts to produce optically active (S)- or (R)-isopropyl glycidyl ether and the corresponding optically active diol

Samples 264-295 in Table 5 illustrate the wild type yeasts identified as capable of producing optically active (S)- or (R)-isopropyl glycidyl ether (GIE) and (S)- or (R)-3-isopropoxy-1,2-propanediol from (±)-isopropyl glycidyl ether.

TABLE 5
Examples of yeast strains from different genera that hydrolyse isopropyl
glycidyl ether enantioselectively^{a}. Positive ee values denote yeast that preferentially
hydrolyse (S)-GIE to produce optically active (R)-GIE while negative ee values denote
yeast that preferentially hydrolyse (R)-GIE to produce optically active (S)-GIE.

| | | | Culture | (S)- | (R)- | | | |
|---|---|---|---|---|---|---|---|---|
| | Screen | | collection | GIPE | GIPE | ees | Conv | Abs |
| No. | no. | Species | nr. | (mM) | (mM) | (%) | (%) | conf |
| 264 | Jen 19 | Arxula adeninivorans | UOFS Y-1220 | 3.66 | 3.94 | 3.7 | 39.2 | R |
| 265 | 752 | Candida magnoliae | UOFS Y-1041 | 1.90 | 3.07 | 23.4 | 60.2 | R |
| 266 | 751 | Candida magnoliae | UOFS Y-1040 | 0.17 | 0.30 | 27.3 | 96.3 | R |
| 267 | 678 | Candida magnoliae | UOFS Y-1297 | 2.15 | 3.51 | 24.0 | 54.7 | R |
| 268 | 677 | Candida magnoliae | UOFS Y-0799 | 2.79 | 3.83 | 15.6 | 47.0 | R |
| 269 | 669 | Candida magnoliae | NCYC 3154 | 4.53 | 4.97 | 4.6 | 24.0 | R |
| 270 | 33 | Candida parapsilosis | UOFS Y-0206 | 5.22 | 5.55 | 3.1 | 13.8 | R |
| 271 | Jen 03 | Cryptococcus albidus | UOFS Y-0821 | 3.32 | 3.69 | 5.3 | 43.9 | R |
| 272 | Jen 12 | Cryptococcus laurentii | NCYC 3160 | 3.74 | 4.04 | 3.9 | 37.8 | R |
| 273 | AB 58 | Cryptococcus podzolicus | NCYC 3164 | 3.95 | 5.32 | 14.7 | 25.9 | R |
| 274 | AB 49 | Cryptococcus podzolicus | UOFS Y-1882 | 3.01 | 4.15 | 15.9 | 42.8 | R |
| 275 | AB 43 | Cryptococcus podzolicus | UOFS Y-1902 | 3.01 | 3.57 | 8.5 | 47.3 | R |
| 276 | 109 | Debaryomyces hansenii | UOFS Y-0613 | 4.06 | 4.45 | 4.5 | 31.9 | R |
| 277 | 113 | Debaryomyces hansenii | NCYC 3167 | 2.35 | 2.70 | 7.0 | 59.6 | R |
| 278 | 707 | Pichia guillermondii | NCYC 3174 | 0.77 | 0.88 | 6.5 | 86.8 | R |
| 279 | 706 | Pichia guillermondii | UOFS Y-0057 | 3.06 | 3.74 | 10.0 | 45.5 | R |
| 280 | 674 | Pichia guillermondii | UOFS Y-1030 | 4.86 | 5.36 | 4.9 | 18.2 | R |
| 281 | 47 | Pichia guillermondii | UOFS Y-1028 | 3.93 | 4.38 | 5.4 | 33.5 | R |
| 282 | 26 | Pichia guillermondii | UOFS Y-0209 | 2.42 | 2.97 | 10.1 | 56.8 | R |
| 283 | 676 | Pichia haplophila | NCYC 3176 | 2.23 | 3.02 | 15.0 | 57.9 | R |
| 284 | 673 | Pichia haplophila | NCYC 3177 | 2.61 | 3.31 | 11.9 | 52.7 | R |
| 285 | 28 | Pichia haplophila | UOFS Y-2136 | 2.08 | 2.63 | 11.8 | 62.3 | R |
| 286 | 112 | Pichia guillermondii | UOFS Y-0053 | 3.12 | 3.61 | 7.4 | 46.2 | R |
| 287 | Car 205A | Rhodosporidium toruloides | UOFS Y-2258 | 4.18 | 5.08 | 9.7 | 26.0 | R |
| 288 | Car 200 | Rhodosporidium toruloides | UOFS Y-2256 | 2.70 | 2.97 | 4.6 | 54.6 | R |
| 289 | 25 | Rhodotorula araucariae | NCYC 3183 | 3.81 | 5.19 | 15.4 | 28.0 | R |
| 290 | 681 | Rhodotorula glutinis | UOFS Y-0653 | 4.33 | 5.21 | 9.2 | 23.7 | R |
| 291 | 6 | Rhodotorula glutinis | UOFS Y-0513 | 3.17 | 4.58 | 18.2 | 38.0 | R |
| 292 | 165 | Rhodotorula sp. | NCYC 3193 | 2.76 | 4.30 | 21.8 | 43.5 | R |
| 293 | 24 | Rhodotorula sp. | UOFS Y-2042 | 1.27 | 5.18 | 60.5 | 48.4 | R |
| 294 | Jen 28 | Sporobolomyces tsugae | NCYC 3199 | 4.98 | 4.67 | −3.2 | 22.8 | S |
| 295 | Jen 48 | Yarrowia lipolytica | UOFS Y-1700 | 3.40 | 3.62 | 3.2 | 43.8 | R |

^{a}Reaction conditions: 50 mM isopropyl glycidyl ether, 50% cells (w/v) in phosphate buffer (50 mM, pH 7.5), Reaction time 3 hours.

Samples 296-297 (FIGS. 10A and 10B) graphically illustrate the hydrolysis of (±)-isopropyl glycidyl ether by selected wild type yeasts to produce optically active (R)-isopropyl glycidyl ether and the corresponding (S)-diol.

Sample 293-294 (FIGS. 11A and 11B) illustrates the profile for the hydrolysis of (±)-isopropyl glycidyl ether by recombinant yeast expression hosts transformed with the epoxide hydrolase genes from selected wild type yeast strains to produce optically active (R)-isopropyl glycidyl ether and the corresponding (S)-3-isopropyloxy-1,2-propanediol.

### Example 11

### Hydrolysis of (±)-Glycidyl tosylate (glycidyl-p-toluenesulfonate) by recombinant yeasts transformed with the epoxide hydrolase genes from selected wild type yeast strains to produce optically active glycidyl tosylate and optically active 3-tosyloxy-1,2-propanediol

Sample 300-301 (FIGS. 12A and 12B) illustrates the profile for the hydrolysis of (±)-isopropyl glycidyl ether by recombinant yeast expression hosts transformed with the epoxide hydrolase genes from selected wild type yeast strains to produce optically active (R)-glycidyl tosylate and the corresponding (S)-3-tosyloxy-1,2-propanediol.

### Example 12

### Hydrolysis of (±)-Naphtyl glycidyl ether (2-[(2-naphthyloxy)methyl]oxirane) by recombinant yeasts transformed with the epoxide hydrolase genes from selected wild type yeast strains to produce optically active glycidyl tosylate and optically active 3-(2-naphtyloxy)-propane-1,2-diol

Sample 302-305 (FIGS. 13A and 13D) illustrates the profile for the hydrolysis of (±)-naphtyl glycidyl ether by recombinant yeast expression hosts transformed with the epoxide hydrolase genes from selected wild type yeast strains to produce optically active (R)-naphtyl glycidyl ether and the corresponding (S)-3-(2-naphtyloxy)-propane-1,2-diol.

### REFERENCES

The following references are included herein by reference thereto.
Botes, A. L. (1999). Affinity purification and characterisation of a yeast epoxide hydrolase. Biotechnol Lett. 21: 511-517.
Botes, A. L., Weijers, C. A., Botes, P. J. and Van Dyk, M. S. (1999). Enantioselectivities of yeast epoxide hydrolases for 1,2-Epoxides. Tetrahedron: Asymmetry 10: 3327-3336.
Choi, W. J., Huh, E. C., Park, H. J., Lee, E. Y. & Choi, C. Y. (1998). Kinetic resolution for optically active epoxides by microbial enantioselective hydrolysis. Biotechnology Techniques, 12: 225-228.
Corey, E. J., Helal, C. J. (1998). Reduction of Carbonyl Compounds with Chiral Oxazaborolidine Catalysts: A New Paradigm for Enantioselective Catalysis and a Powerful New Synthetic Method. Angewandte Chemie International Edition 37 (15) 1986-2012.
Gong, P-F. & Xu, J-H. (2005). Bio-resolution of a chiral epoxide using whole cells of Bacillus megaterium ECU 1001 in a biphasic system. Enzyme and Microbial Technology 36: 252-257.
Kolb, H. C., van Nieuwenhze, M. S. and Sharpless, K. B. (1994). Catalytic asymmetric dihydroxylation. Chem. Rev. 94: 2483-2547.
Lutje Spelberg, J. H., Rink, R., Kellogg, R. M. & Janssen, D. B. (1998). Enantioselectivity of a recombinant epoxide hydrolase from Agrobacterium radiobacter. Tetrahedron: Asymmetry 9: 459-466.
Madzak, C., Gaillardin, C., Beckerich, J-M. (2004). Heterologous protein expression and secretion in the non-conventional yeast Yarrowia lipolytica: a review. Journal of Biotechnology 109: 63-81.
Martínez, F., Del Campo, C., Sinisterra, J. V. & Llama, E. F. (2000). Preparation of halohydrin β-blocker precursors using yeast-catalysed reduction. Tetrahedron: Asymmetry 11: 4651-4660.
Peukert, S. & Jacobsen, E. N. (1999). Enantioselective parallel synthesis using polymer-supported chiral Co (salen) complexes. Organic Letters 1 (8): 1245-1248.
Prichanont, S., Leak, D. J. & Stuckey, D. C. (1998). Alkene monooxygenase-catalyzed whole cell epoxidation in a two-liquid phase system. Enzyme and Microbial Technology 22: 471-479.
Reetz, M. T., Torre, C., Eipper, A., Lohmer, R., Hermes, M., Brunner, B., Maichele, A., Bocola, M., Arand, M., Cronin, A., Genzel, Y., Archelas, A. and Furstoss, R. (2003). Enhancing the enantioselectivity of an epoxide hydrolase by directed evolution. Organic Letters, 6: 177-180.
Salazar, L., Bermudez, J. L., Ramirez, C., Llama, E. F. & Sinisterra, J. V. (1999). Resolution of 3-α-naphtoxy-1,2-propanediol using Candida antarctica lipase. Tetrahedron: Asymmetry 10: 3507-3514.
Steinreiber, A., Osprian, I., Mayer, S. F., Orru, R. V. and Faber, K. (2000). Enantioselective hydrolysis of functionalized 2,2-disubstituted oxiranes with bacterial epoxide hydrolases. Eur. J. Org. Chem. 22: 3703-3711.
Subhas Bose, D. & Venkat Narsaiah, A. (2005). An efficient asymmetric synthesis of (S)-atenolol: using hydrolytic kinetic resolution. Bioorganic & Medicinal Chemistry 13: 627-630.
Tang, Y-F., Xu, J-H., Ye, Q. & Schulze, B. (2001). Biocatalytic preparation of (S)-phenyl glycidyl ether using newly isolated Bacillus megaterium ECU 1001. Journal of Molecular Catalysis B: Enzymatic 13: 61-68.
Van Loo, B., Lutje Spelberg, J. H., Kingma, J., Sonke, T., Wubbolts, M. & Janssen, D. B. (2004). Directed evolution of epoxide hydrolase from A. radiobacter towards higher enantioselectivity by error-prone PCR and DNA shuffling. Chemistry & Biology, 11: 981-990.
Visser, H., Weijers, C. A. G. M., Van Ooyen, A. J. J. and Verdoes, J. C. (2002). Cloning, characterisation and heterologous expression of epoxide hydrolase-encoding cDNA sequences from yeasts belonging to the genera Rhodotorula and Rhodosporidium. Biotechnol. Lett. 24:1687-1694.
Wang, Z-M., Zhang, X-L. & Sharpless, K-B. (1993). Asymmetric dihydroxylation of aryl allyl ethers. Tetrahedron Letters 34: 2267-2270.
Weijers, C. A. G. M. & De Bont, J. A. M. (1999) Epoxide hydrolases from yeasts and other sources: versatile tools in biocatalysis. Journal of Molecular Catalysis B: Enzymatic, 6: 199-214.
Xu, Y., Xu, J-H., Pan, J. & Tang, Y-F. (2004). Biocatalytic resolution of glycidyl aryl ethers by Trichosporon loubierii: cell/substrate ratio influences the optical purity of (R)-epoxides. Biotechnology Letters 26:1217-1221.
Xu, Y., Xu, J-H., Pan, J., Zhao, L. & Zhang, S-L. (2004). Biocatalytic resolution of nitro-substituted phenoxypropylene oxides with Trichosporon loubierii epoxide hydrolase and prediction of their enantiopurity variation with reaction time. Journal of Molecular Catalysis B: Enzymatic, 27:155-159.
Kotik, M., Brichac, J., Kyslik, P. (2005). Novel microbial epoxide hydrolases for biohydrolysis of glycidyl derivatives. Journal of Biotechnology (article in press).

A number of embodiments of the invention have been described. Nevertheless, it will be understood that various modifications can be made without departing from the spirit and scope of the invention. Accordingly, other embodiments are within the scope of the following claims.

1. A process for obtaining at least one of an optically active glycidyl ether and an optically active vicinal diol, which process includes the steps of:
providing an enantiomeric mixture of a glycidyl ether;
creating a reaction mixture by adding to the enantiomeric mixture a polypeptide, or a functional fragment thereof, having enantioselective glycidyl ether hydrolase activity, the polypeptide being a polypeptide encoded by a gene of a yeast cell;
incubating the reaction mixture; and
recovering from the reaction mixture at least one of an enantiopure, or a substantially enantiopure, vicinal diol, and an enantiopure, or a substantially enantiopure, glycidyl ether.

2. A process for obtaining at least one of an optically active glycidyl ether and an optically active vicinal diol, which process includes the steps of:
providing an enantiomeric mixture of a glycidyl ether;
creating a reaction mixture by adding to the enantiomeric mixture a cell comprising a nucleic acid encoding, and capable of expressing, a polypeptide having enantioselective glycidyl ether hydrolase activity;
incubating the reaction mixture; and
recovering from the reaction mixture at least one of an enantiopure, or a substantially enantiopure, vicinal diol, and an enantiopure, or a substantially enantiopure, glycidyl ether.

3. The process of claim 2, wherein the cell is a yeast cell.

4. The process of claim 2, wherein the polypeptide is encoded by an endogenous gene of the cell.

5. The process of claim 2, wherein the cell is a recombinant cell and the polypeptide is encoded by a nucleic acid sequence with which the cell is transformed.

6. The process of claim 5, wherein the nucleic acid sequence is a heterologous nucleic acid sequence.

7. The process of claim 5, wherein the nucleic acid sequence is a homologous nucleic acid sequence.

8. The process of any claim 1, wherein the polypeptide is a full-length yeast epoxide hydrolase.

9. The process of claim 1, wherein the polypeptide is a functional fragment of yeast epoxide hydrolase.

10. The process of claim 1, wherein the process is carried out at a pH from 5 to 10.

11. The process of claim 1, wherein the process is carried out at a temperature of 0° C. to 70° C.

12. The process of claim 1, wherein the concentration of the glycidyl ether in the reaction mixture is at least equal to the soluble concentration of the glycidyl ether in water.

13. The process of claim 1, wherein the glycidyl ether of the enantiomeric mixture and the obtained optically active epoxide is a compound of the general formula (I) and the vicinal diol produced by the process is a compound of the general formula (II), 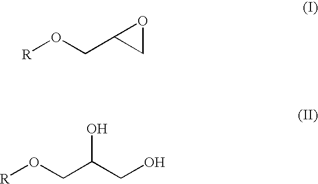
wherein, R represents a variably substituted straight-chain or branched alkyl group, a variably substituted straight-chain or branched alkenyl group, a variably substituted straight-chain or branched alkynyl group, a variably substituted cycloalkyl group as well as cycloalkenyl groups, a variably substituted aryl group, a variably substituted aryl alkyl group, a variably substituted heterocyclic group, a variably substituted alkylthio group, a variably substituted alkoxycarbonyl group, a variably substituted straight chain or branched alkylamino or alkenyl amino group, a variably substituted arylamino or arylalkylamino group, a variably substituted carbamoyl group, a variably substituted acyl group or a functional group

14. The process of claim 13, wherein the alkyl group is a straight chain or branched alkyl group with 1 to 12 carbon atoms.

15. The process of claim 13 wherein the alkenyl group is a straight chain or branched alkenyl group with 2 to 12 carbon atoms.

16. The process of claim 13, wherein the alkynyl group is a straight chain or branched alkynyl group with 2 to 12 carbon atoms

17. The process of claim 13, wherein the cycloalkyl group is a cycloalkyl group with 3 to 10 carbon atoms.

18. The process of claim 13, wherein the cycloalkenyl group is a cycloalkenyl group with 3 to 10 carbon atoms.

19. The process of claim 13, wherein the aryl group is a phenyl, biphenyl, naphtyl, or anthracenyl group.

20. The process of claim 13, wherein the aryl alkyl group is an aryl alkyl group with 7 to 18 carbons.

21. The process of claim 13, wherein the heterocyclic group is a 5 to 7-membered heterocyclic group containing nitrogen, oxygen or sulphur fused with a cyclic or aromatic ring having 3 to 7 carbon atoms.

22. The process of claim 13, wherein the alkylamino group is a straight chain or branched alkylamino group with 2 to 12 carbon atoms.

23. The process of claim 13, wherein the arylamino group is an arylamino group which can be substituted with an alkyl, alkenyl or alkoxy group having 1 to 4 carbon atoms.

24. The process of claim 13, wherein the alkylamino group is benzylamino or 2-phenylethylamino.

25. The process of claim 13, wherein the alkylthio group is an alkylthio group having 1 to 8 carbon atoms.

26. The process of claim 13, wherein the alkenylthio group is a straight chain or branched alkenylthio group having 1 to 8 carbon atoms.

27. The process of claim 13, wherein the arylthio group is an arylthio group having 1 to 8 carbon atoms which can be substituted with an alkyl or alkenyl or alkoxy group having 1 to 4 carbon atoms.

28. The process of claim 13, wherein the arylalkylthio group is an arylalkylthio group having 1 to 8 carbon atoms.

29. The process of claim 13, wherein the substituted or unsubstituted carbamoyl group is selected from carbamoyl, methylcarbamoyl, dimethylcarbamoyl, diethylcarbamoyl and dipropylcarbamoyl.

30. The process of claim 13, wherein the acyl group is an acyl group with 1 to 8 carbon atoms.

31. The process of claim 13, wherein R takes the form of R′—X, where X is a functional group bonded to any carbon of R′ except C₁.

32. The process of claim 13, wherein —OR as a whole is replaced by a functional group

33. The process of claim 1, wherein the enantiomeric mixture is a racemic mixture.

34. The process of claim 1, which process includes adding to the reaction mixture water and at least one water-immiscible solvent.

35. The process of claim 1, which process includes adding to the reaction mixture water and at least one water-miscible organic solvent.

36. The process of claim 1, which process includes stopping the reaction when one enantiomer of the glycidyl ether and/or vicinal diol is in excess compared to the other enantiomer of the glycidyl ether and/or vicinal diol.

37. The process of claim 1, which process includes recovering continuously during the reaction the optically active epoxide and/or the optically active vicinal diol produced by the reaction directly from the reaction mixture.

38. The process of claim 1, wherein the yeast cell is of a yeast genus selected from the group consisting of Arxula, Brettanomyces, Bullera, Bulleromyces, Candida, Cryptococcus, Debaryomyces, Dekkera, Exophiala, Geotrichum, Hormonema, Issatchenkia, Kluyveromyces, Lipomyces, Mastigomyces, Myxozyma, Pichia, Rhodosporidium, Rhodotorula, Sporidiobolus, Sporobolomyces, Trichosporon, Wingea, and Yarrowia.

39. The process of claim 1, wherein the yeast cell is of a yeast species selected from the group consisting of Arxula adeninivorans, Arxula terrestris, Brettanomyces bruxellensis, Brettanomyces naardenensis, Brettanomyces anomalus, Brettanomyces species (e.g. Unidentified species NCYC 3151), Bullera dendrophila, Bulleromyces albus, Candida albicans, Candida fabianii, Candida glabrata, Candida haemulonii, Candida intermedia, Candida magnoliae, Candida parapsilosis, Candida rugosa, Candida tenuis, Candida tropicalis, Candida famata, Candida kruisei, Candida sp. (new) related to C. sorbophila, Cryptococcus albidus, Cryptococcus amylolentus, Cryptococcus bhutanensis, Cryptococcus curvatus, Cryptococcus gastricus, Cryptococcus humicola, Cryptococcus hungaricus, Cryptococcus laurentii, Cryptococcus luteolus, Cryptococcus macerans, Cryptococcus podzolicus, Cryptococcus terreus, Debaryomyces hansenii, Dekkera anomala, Exophiala dermatitidis, Geotrichum spp. (e.g. Unidentified species UOFS Y-0111), Hormonema spp. (e.g. Unidentified species NCYC 3171), Issatchenkia occidentalis, Kluyveromyces marxianus, Lipomyces spp. (e.g. Unidentified species UOFS Y-2159), Lipomyces tetrasporus, Mastigomyces philipporii, Myxozyma melibiosi, Pichia anomala, Pichia finlandica, Pichia guillermondii, Pichia haplophila, Rhodosporidium lusitaniae, Rhodosporidium paludigenum, Rhodosporidium sphaerocarpum, Rhodosporidium toruloides, Rhodosporidium paludigenum, Rhodotorula araucariae, Rhodotorula glutinis, Rhodotorula minuta, Rhodotorula minuta var. minuta, Rhodotorula mucilaginosa, Rhodotorula philyla, Rhodotorula rubra, Rhodotorula spp. (e.g. Unidentified species NCYC 3193, UOFS Y-2042, UOFS Y-0448, UOFS Y-0139, UOFS Y-0560), Rhodotorula aurantiaca, Rhodotorula spp. (e.g. Unidentified species NCYC 3224), Rhodotorula sp. “mucilaginosa”, Sporidiobolus salmonicolor, Sporobolomyces holsaticus, Sporobolomyces roseus, Sporobolomyces tsugae, Trichosporon beigelii, Trichosporon cutaneum var. cutaneum, Trichosporon delbrueckii, Trichosporon jirovecii, Trichosporon mucoides, Trichosporon ovoides, Trichosporon pullulans, Trichosporon spp. (e.g. Unidentified species NCYC 3210, NCYC 3212, NCYC 3211, UOFS Y-0861, UOFS Y-1615, UOFS Y-0451, UOFS Y-0449, UOFS Y-2113), Trichosporon moniliiforme, Trichosporon montevideense, Wingea robertsiae, and Yarrowia lipolytica.

40. A method for producing a polypeptide, which process includes the steps of:
providing a cell comprising a nucleic acid encoding and capable of expressing a polypeptide that has enantioselective glycidyl ether hydrolase activity;
culturing the cell; and
recovering the polypeptide from the culture.

41. The method of claim 40, wherein the cell is a yeast cell.

42. The method of claim 40, wherein the polypeptide is a full-length yeast epoxide hydrolase.

43. The method of claim 40, wherein the polypeptide is a functional fragment of a yeast epoxide hydrolase.

44. The method of claim 40, wherein the polypeptide is encoded by an endogenous gene of the cell.

45. The method of claim 40, wherein the cell is a recombinant cell and the polypeptide is encoded by a nucleic acid sequence with which the cell is transformed.

46. The method of claim 45, wherein the nucleic acid sequence is a heterologous nucleic acid sequence.

47. The method of claim 45, wherein the nucleic acid sequence is a homologous nucleic acid sequence.

48. A crude or pure enzyme preparation which includes an isolated polypeptide having enantioselective glycidyl ether hydrolase activity.

49. A substantially pure culture of cells, a substantial number of which comprise a nucleic acid encoding, and are capable of expressing, a polypeptide having enantioselective glycidyl ether hydrolase activity.

50. An isolated cell, the cell comprising a nucleic acid encoding a polypeptide having enantioselective glycidyl ether hydrolase activity, the cell being capable of expressing the polypeptide.

51. An isolated DNA comprising:
(a) a nucleic acid sequence that encodes a polypeptide that has enantioselective glycidyl ether hydrolase activity and that hybridizes under highly stringent conditions to the complement of a sequence selected from the group consisting of SEQ. ID. NOs: 10, 11, 12, 13, 14, 15, 16, 17 and 18; or
(b) the complement of the nucleic acid sequence.

52. The DNA of claim 51, wherein the nucleic acid sequence encodes a polypeptide comprising an amino acid sequence selected from the group consisting of SEQ. ID. NOs: 1, 2, 3, 4, 5, 6, 7, 8 and 9.

53. The DNA of claim 51, wherein the nucleic acid sequence is selected from the group consisting of SEQ ID NOs: 10, 11, 12, 13, 14, 15, 16, 17 and 18.

54. An isolated DNA comprising:
(a) a nucleic acid sequence that is at least 55% identical to a sequence selected from the group consisting of SEQ ID NOs: 10, 11, 12, 13, 14, 15, 16, 17 and 18; or
(b) the complement of the nucleic acid sequence,
wherein the nucleic acid sequence encodes a polypeptide that has enantioselective glycidyl ether hydrolase activity.

55. An isolated DNA comprising;
(a) a nucleic acid sequence that encodes a polypeptide consisting of an amino acid sequence that is at least 55% identical to a sequence selected from the group consisting of SEQ ID NOs: 1, 2, 3, 4, 5, 6, 7, 8 and 9; or
(b) the complement of the nucleic acid sequence,
wherein the polypeptide has enantioselective glycidyl ether hydrolase activity.

56. An isolated polypeptide encoded by the DNA of claim 51.

57. An isolated polypeptide comprising an amino acid sequence that is at least 55% identical to SEQ. ID. NOs: 1, 2, 3, 4, 5, 6, 7, 8 or 9, the polypeptide having enantioselective glycidyl ether hydrolase activity.

58. The polypeptide of claim 57, comprising:
(a) an amino acid sequence selected from the group consisting of SEQ. ID. NOs; 1, 2, 3, 4, 5, 6, 7, 8 and 9 or a functional fragment of the sequence; or
(b) the sequence of (a), but with no more than five conservative substitutions,
wherein the polypeptide has enantioselective glycidyl ether hydrolase activity.

59. An isolated antibody that binds to the polypeptide of claim 56.

60. The antibody of claim 59, wherein the antibody is a polyclonal antibody.

61. The antibody of claim 59, wherein the antibody is a monoclonal antibody.


SEQUENCE LISTING (37 sequences)

SEQ ID NO: 1 - protein, 409 aa, Rhodosporidium toruloides NCYC 3181
MATHTFASPPTRFTVDIPQSELDELDFRLDKTRWPATEIVPEDGADDPTAFGLGAGPTLPLMKELAKGWREFDWKKAQDHLNTFEHYTVEIEDLSIHFLHHRSTRPNAVPLILCHGWPGHFGEFLNVIPLLTEPSDPSAQAFHVVAPSMPGYAWSSPPPSSKWSMPDTARVFDKLMTGLGYEKYMAQGGDWGSIAARCLGSLHKDHCKAVHLNFLPVFPPVPMWLINPHTLLAWAPRFLVPEKQAARMKRGLAYLEKGSAYYVMQQLTPRTPAYGLTDSPVGLLAWIGEKFEPTIQEASKQAQPTLTRDELYFTCSLYWFTRSIGTSFLPYSLNPHFTTFLTDSRYHLPNFALSLYPGEIYCPAERDAKRTGNLKWIKDAPDGGHFAALEKPDVFVEHLREAFGVMWEK

SEQ ID NO: 2 - protein, 409 aa, Rhodosporidium toruloides UOFS Y-0471
MATHTFASPPTRFTVDIPQSELDELHSRLDKTRWPATEIVPEDGTDDPTAFGLGAGPTLPLMKELAKGWREFDWKKAQDHLNTFEHYMVEIEDLSIHFLHHRSTRPNAVPLILCHGWPGHFGEFLNVIPLLTEPSDPSAQAFHVVAPSMPGYAWSLPPPSSKWNMPDTARVFDKLMTGLGYEKYMAQGGDWGSIAARCLGSLHKDHCKAVHLNFLPVFPPVPMWLINPHTLLAWAPRFLVPEKQAARMKRGLAYLEKGSAYYVMQQLTPRTPAYGLTDSPVGLLAWIGEKFEPTIQEASKQAQPTLTRDELYFTCSLYWFTRSIGTSFLPYSLNPHFTTFLTDSKYHLPNFALSLYPGEIYCPAERDAKRTGNLKWIKDAPEGGHFAALEKPDVFVEHLREAFGVMWEK

SEQ ID NO: 3 - protein, 411 aa, Rhodosporidium paludigenum NCYC 3179
MAAHSFTAPPAPYNIDFAPQVNDLHRRLDAARWPEHDVVPDDVDHGEHGAFGLGAGPSLALMKELAQEWRGQDQKQLQDHLNSYKNYRVEIEGLNIHFLHYPSSRADAFPLILCHGWPGGYHEFLHVLERLTEPEDQGSRAFHVVVPSMPGYAFSSPPKTAKWGMEDTARVFDKLMTGLGYAKYAAQGGDWGSITARCLGSLHKENCVAVHLNFCPVPPPFPLNMFNPRTLLDWMPRFVLPDQRRAKIERGVAYIERGSAYYAMQNLTPRTPAYGLNDSPIGLLAWIGEKMIPGIDKAVKHPNATLNREALFTTLSIYWFTGSIGSSFLPYALNPHFSTFLVSPRHQLPNFALSNFPDELFTPEERDARRTGNLRWYKDAEDGGHFAALEKPEVFAEHVREAMGVLLSNQA

SEQ ID NO: 4 - protein, 410 aa, Rhodotorula araucariae NCYC 3183
MSEHSFEAPPQPFTVDFAPHIEDLHRRLDNARWPTQEIVPVDVSETEHHNAFGLGMGPQLNLMKELANGWRAFDQSALQDHLNSFNNWKVEIEGLSIHFLHHRSTRAGALPLILCHGWPGGYHEFLHVVQLLTEPEGADAQAFHLVVPSMPGYAFSSPPPTAKWGMEDTARVFDKLMTGLGYNKYVAQGGDWGSITARCLGALHKDHCIAVHLNFCPVPPPFPFDQFNPRTLLNWMPRFVISDQQRAKLERGLAYLEKGSAYYVMQQLTPRTPAYALNDSPIGLLAWIGEKMIPGINEASAQPNPTLNRDALYTTLSLYWFTNSIGTSFLPYSLNPHFSTFLTSPRYRLPNFALSSFPGELFSPTPRDAARTGVMRWYKEADDGGHFAALEKPDVFSQHVREAVKAMLSS

SEQ ID NO: 5 - protein, 409 aa, Cryptococcus curvatus NCYC 3158
MATHTFASPPTRFTVDIPQSEVDQLHSRLDKTRWPGTEIVPEDGADDPTAFGLGAGPTLPLMKELAKGWRDFDWKKAQDHLNTFEHYTVEIEDLSIHFLHHRSTRPNAVPLILCHGWPGHFGEFLHVIPLLTEPSDPSAQAFHVVAPSMPGYAWSSPPPTSKWNMPDTARVSDKLMNGLGYEKYMAQGGDWGSIAARCLGALHKDHCKAVHLNFLPVFPPVPMWLINPHTLLAWAPRFLVPEKQAARMKRGLAYLEKGSAYYVMQQLTPRTSAYGLTDSPVGLLAWIGEKFEPTIQEASKQAQPTLTRDELYFTCSLYWFTRSIGTSFLPYSLNPHFTTFLTDSKYYLPNFALSLYPGEIYCPAERDAKRTGNLKWIKDAPEGGHFAALEKPDVFVDHLREAFGVMWEK

SEQ ID NO: 6 - protein, 354 aa, Yarrowia lipolytica NCYC 3229
MTDSLSLGYYQLFHKYAVLGDKRWHYLDIPPTSPSPALLGSGKTLMLCHGFPDSWYGWRKQIPILRNLGYRLLVPSQMGYTRSEAPLYPTPEPNEKGEYPELGLEDGTDALKDLYCYTGKFYAQCMDELLTQLGIPTVTIIGHDYGAYLGPKLYLYFPHRVEAIATSCWHYVPALKKFFAIPDFIKLAPSLSYQAYLIGDASKDFATREGSEPFLRQVFGGEGWGGSDSSVHMTEPEFQNYMTEFATGGRFLSYMVSTYKARRLTFEIEKQDFLDKGRTPESMQVDVPYLHVGAEQDMAFRPPMIKNLRKYVKPGRLDEVWIDASHWLMFEKADEFNHELVKWLDKIHGSQSKL

SEQ ID NO: 7 - protein, 348 aa, Yarrowia lipolytica NCYC 3229
MSSLDLSYYEPFRRNAVLNGKRWHYVDIPGDSSGSLGRGKTLLLVHGFPDTWYGWRHQVPVFRKHGFRLLIPSLLGFPRSEAPLTHPGVATGEKFDGHNVHKELGLEDENIQELECYTADFFAKSMVQLLDQLGIDKVCSFGHDWGAVFAPRLWLNYPERVECVSSACWYYQMPMEGFVDLKDFAEVAPSLRYQLYWGGDAPQEVIQKPLKEFMDRMYHHPTENHLLTSEAEYNNILKEFQYGGKTIAPMFPLYKSRKLAYDIDERDFLTKGRTEEDLAVDVPYLFIGAEFDIALQPGMESVLEGYVKEGLLEKQWVPCGHWVLFEEPEKMNKIYIDFLKKVFGSSKL

SEQ ID NO: 8 - protein, 401 aa, Filobasidiella (Cryptococcus)  neoformans var eofs
MSYSDLPHKPTIPVEPFKLSVPHEDLNGLLTLLKSTRIAKESYENVSAHENKFGITRKWLVNMKDEWIKQDWRKQEERINSLPAFKAKVKNSDGSVFSIHFTALFSKKKVAIPIILSHGWPGSFYEFVPMMEMVKKKYSPEDLPFHLIVPSLPGWLFSTPPPNDREFNVTDVGYLFNGLMEGLGFGDGYVAQGGDIGSYVTNELGAKYPACKIIHVNYSNPPPRPLPSPGSPGQEASPPSAEDLLELLQKFGYALEHSTRPATVGLVVGSNPLSLLAWVGEKFLEWTDESPSEETILTMTSLYWFTDCFTTSIYTYRYGLGAKRHESAKQASYQKCPLGYSQFPKEIVEIPAEWVKAQSNMVWSKKHESGGHFAALEKPELLWADIEEFVHSQWENYKGNY

SEQ ID NO: 9 - protein, 394 aa, Rhodotorula mucilaginosa  NCYC 3190
MPARSLTLRPFSPSFTAPELDGLARSLESSRLPAETYASRQAKYGIKHAWMKNALQRWKDGFDWKKHEQDINEVDHYMVQVQSDGIQHDLHVIYHESKDPNAIPLLLLHGWPGSAFEFIEAIKILRKSTSPAFHLIAPMEPGYGWSTPPPLDRGFNMNDCTALMNDLMVGLGYGDGYAAQGGDIGSGLARLLAVNYDACKCININYMPAVAPPEDAPERHQIKPHEEDALRRADEFQKTGRGYANMHATRPGTVGIVVGSSPVALLAWIAEKYLAWTDEDPPLDTILAICTIWWIRDSYPSSIWAYADFLETGISALHNDPKYKLDKKPFGFSSFKEEISATPEAWAGRNGNLQFYRYHDKGGHFAALEQPEAFAQDMQDCFGKIWPLSQEQKS

SEQ ID NO: 10 - DNA, 1230 bp, Rhodosporidium toruloides NCYC 3181
atggcgacacacacattcgcttcgcctcccacgcgcttcaccgtcgacatcccacagtcagaactcgacgaactcgacttccgactcgacaagacccgctggccggcgacagagatcgttccagaggatggggcggacgacccgacggcgtttgggctcggagcagggccgacgctgccgctcatgaaggaactggcgaagggttggcgcgagttcgactggaagaaggcgcaggaccacctcaacacctttgagcactacacggtcgaaatcgaggacctctccatccacttcctccaccaccgctcgactcgcccgaatgctgttccgctcatcctctgccacggctggccaggccacttcggcgagttcctgaacgtcataccgctcttgacggagccgtcggacccgtccgcccaggcgttccacgtcgtcgcgccttcgatgcccggttatgcttggtcttcgcctcctccgtcctccaagtggagcatgcctgataccgcgagggtcttcgacaagctcatgaccgggcttggctacgagaagtacatggcgcagggcggagactggggcagcatcgctgctcgctgccttggatcgcttcacaaagaccactgcaaagccgtccacctcaacttcctccccgtcttcccacccgtcccgatgtggcttatcaacccgcacacgctccttgcctgggcaccgcgcttcctcgtgccggagaagcaggctgcgcgtatgaagcgcgggttggcgtaccttgagaagggctccgcctactacgtcatgcagcagttgacgcctcgcacgcctgcgtacggcctgaccgacagtcccgtcggcttgctggcctggatcggcgagaagttcgagccgaccattcaggaggcgagcaagcaagcccagccgaccctgactcgcgacgagctctacttcacctgctcgctctactggttcacccgctcaatcggcacctccttccttccctactcgctcaacccgcacttcaccaccttcctgaccgacagcaggtaccacctgcccaactttgccctgtccctctacccgggcgagatctactgcccggcagaacgggacgccaagcgtaccggcaacctcaagtggatcaaggacgcgcccgatggaggacactttgctgcgctcgagaagcccgatgtgtttgtcgagcatctcagggaggcgtttggcgtcatgtgggagaagtag

SEQ ID NO: 11 - DNA, 1230 bp, Rhodosporidium toruloides UOFS Y-0471
atggcgacacacacattcgcttcgcctcccacccgcttcaccgtcgacatcccacagtcggaactcgacgaacttcactcgcgactcgacaagacccgctggccggcgacagagatcgttccagaggatgggacggacgatccgacggcgtttgggctcggagcagggccgacgctgccgctcatgaaggaattggcgaagggttggcgcgagttcgactggaaaaaggcgcaggaccacctcaacaccttcgagcactacatggtcgaaattgaggacctctcgatccacttcctccaccatcgctcgactcgcccgaacgctgttcccctcatcctctgccacggctggccaggccactttggcgagttcctgaacgttatcccgctcttgacggagccgtcggacccctccgctcaggcgttccacgtcgtcgccccttcgatgcctggctatgcttggtctttgcctcctccgtcctccaagtggaacatgcctgacaccgcgagggtcttcgacaagctcatgaccgggcttggctacgagaagtacatggcgcagggcggagactggggaagcatcgccgctcgctgccttggatcgctgcacaaggaccattgcaaagccgtccacctcaacttcctccccgtcttcccacccgtcccgatgtggcttatcaacccgcacacgctccttgcctgggcaccgcgcttcctcgtgccggagaagcaggctgcgcgtatgaagcgcgggttggcgtaccttgagaagggctccgcctactacgtcatgcagcagttgacgcctcgcacgcctgcgtacggcctgaccgacagtcccgtcggcttgctggcctggatcggcgagaagttcgagccgaccattcaggaggcgagcaagcaagcccagccgaccctgactcgcgacgagctctacttcacctgctcgctctactggttcacccgctcaatcggcacctccttccttccctactcgctcaacccgcacttcaccaccttcctgaccgacagcaagtaccacctgcccaactttgccctctcgctttacccaggcgagatctactgccccgccgagcgggacgccaagcgcaccggcaacctcaagtggatcaaggacgcgcctgagggaggacactttgctgcgctcgaaaagccggatgtgtttgtcgagcacctcagggaggcgtttggcgtcatgtgggagaagtag

SEQ ID NO: 12 - DNA, 1236 bp, Rhodosporidium paludigenum NCYC 3179
atggctgcccattcctttactgcacctcctgcaccctacaacatcgactttgcgccccaggtaaatgacctgcaccgccgtctcgatgctgcccgctggccggaacacgacgtggtgcccgacgatgtggatcacggagagcacggcgcattcggactcggcgctggtcccagcctcgccctcatgaaggagctcgcgcaggaatggaggggccaggaccagaagcagctgcaggaccacctcaactcctacaagaactatcgcgtcgagatcgagggtctcaacatccacttcctgcactacccgtcgtctcgcgccgatgcgttcccgctcatcctgtgccacggctggcctggcggctaccacgagttcctgcacgtcctagagcgcctcacggagcccgaggatcaggggtcgcgggccttccatgtcgtcgtgccttccatgccgggttacgccttctcctcgccgcccaagacggcaaaatggggcatggaggacacggctcgcgtgttcgacaagctcatgacggggctaggttacgccaagtatgcggcccaaggcggtgactgggggtctatcacggcgcgctgcctaggttcgctgcacaaggagaactgcgtcgctgtccacctcaacttctgcccggttcccccgccgttcccgctcaacatgttcaacccgcgcacacttctggactggatgcctcgctttgtcctgcctgatcaacggcgggccaagattgagcgcggcgtggcctatatcgagcgcggctctgcctactacgccatgcaaaacttgacgccgcgcacgcctgcgtacggcttgaacgatagtccgattggtttgctcgcgtggattggcgagaagatgattccgggcattgacaaggctgtcaagcatccgaacgcaaccctcaatcgcgaagctcttttcacgacactctcgatctactggttcacgggctcgattggctcctccttcctgccatacgctctcaacccgcacttctctaccttcctcgtctcgccgcggcaccaactgccgaactttgctctgtccaactttcccgacgagctgttcacgcccgaagaacgcgatgctcgccgaaccggaaacttgcggtggtacaaggatgcagaggatggagggcacttcgcggcgctggagaagcccgaggtcttcgccgagcacgtaagggaggcgatgggggtcttgctgtcgaaccaggcctga

SEQ ID NO: 13 - DNA, 1233 bp, Rhodotorula araucariae  NCYC 3183
atgagcgagcacagcttcgaggccccgccacagccgtttacggtcgactttgctccccacatcgaggatctccaccgccgtctcgacaatgcgcgctggccgacgcaagagattgtccccgtcgacgtgtccgagacggagcatcacaacgcgttcggactcgggatgggcccgcagctcaaccttatgaaggagctcgccaacggctggcgcgcgttcgaccagtcggcgctccaggaccacctcaacagcttcaacaactggaaggtcgagatcgagggattgtcgatccacttcctccaccatcgctcgacgcgcgccggcgctctcccgctcatcctgtgccatggctggcccggcgggtaccacgagttcctccacgtcgtccagctcctcaccgaaccagagggggcggatgcgcaggcgtttcacctcgtcgtcccctcgatgcccgggtacgccttctcgtctccgccgccgacggccaagtggggcatggaagacactgcaagggtttttgacaagctcatgaccggtttggggtacaacaagtatgtcgcgcagggcggtgactgggggtccatcacggcgcgatgcctcggcgcgctgcacaaggaccactgcattgctgtccacctcaacttctgccccgtcccgccgccgttcccattcgaccagttcaacccgcgcacgctgctcaactggatgccgcgcttcgtgatctcggaccagcagcgtgcgaagctcgagcgtgggctggcgtacctcgagaaggggtctgcttactatgtcatgcagcagctcacaccgcgtaccccggcctacgctctcaatgacagcccgattggcctgctcgcctggattggcgaaaagatgatcccaggcatcaacgaggcgagcgcgcagccgaacccgacgctcaatcgcgatgcgttgtacaccacgctctcgctgtactggttcaccaactccatcggcacctctttcctcccctactcgcttaacccgcacttcagcacgttcctcacctcgccccgctatcgcctgccgaactttgcgctgtcttccttcccgggcgagctgttctcgccgacgccgcgcgatgctgcgaggacgggcgtgatgcgctggtacaaggaggcggacgatggcgggcactttgcggcgctcgagaagcccgatgtgttcagccagcatgtcagggaggcagtcaaggccatgctgtcgtcgtga

SEQ ID NO: 14 - DNA, 1230 bp, Cryptococcus curvatus NCYC 3158
atggcgacacacacattcgcttcgcctcccacccgcttcaccgtcgacattccgcagtcggaagttgaccaacttcactcgcgactcgataagactcgctggccagggacagagatcgttccagaggatggggcggacgacccgacggcgtttggactcggagcaggaccaacgctgccgctcatgaaggaactggcgaagggttggcgcgacttcgactggaagaaggcgcaggaccacctcaacaccttcgagcactacacggtggagatcgaggacctctcgatccacttcctccaccatcgctcgactcgtccgaatgctgttcccctcatcctctgccacggctggccaggacacttcggcgagtttctacacgttataccactcttgacggagccgtcggacccgtccgctcaggcgtttcacgtcgtcgcgccttcgatgcctggttatgcttggtcttcgcctcctcctacctccaagtggaatatgcctgacaccgcaagggtgtccgacaagcttatgaacggactcggctacgagaagtacatggcgcagggcggagactggggcagcatcgccgctcgctgcctcggagcgctgcacaaagatcactgcaaagccgtccacctcaacttcctccccgtcttcccgcccgtccctatgtggctcatcaacccacacacactcctcgcatgggctccgcgcttccttgtgccggagaagcaggctgcgcgcatgaagcgcgggttggcgtacctcgaaaagggctccgcctactacgtcatgcagcaattgacgcctcgcacgtctgcgtacggccttactgacagtcccgtcggcttgctggcctggatcggcgagaagtttgagccgaccattcaggaagcgagcaagcaagcccagccgaccctcactcgcgacgagctctacttcacctgctccttgtactggttcacccgctcaatcggcacctccttcctgccctactcgctcaacccgcacttcacgaccttcctgaccgacagcaagtattacctgcccaattttgccctctccctgtacccgggcgagatctactgccctgccgagcgggacgccaagcgcactggcaacctcaagtggatcaaggacgcgcctgagggaggacactttgcggcgctcgagaagcccgacgtgtttgtcgaccatctcagggaggcatttggcgttatgtgggagaagtag

SEQ ID NO: 15 - DNA, 1065 bp, Yarrowia lipolytica NCYC 3229
atgaccgactccctctcgctcggatactaccaactgttccacaagtacgccgttctcggagacaaaagatggcactatctcgacattccacccaccagtccgagccccgctcttcttggatccggaaagaccctgatgctgtgccacggcttccccgatagctggtatggatggcgaaaacagattcccatactgcgcaaccttggctacagactcctagtaccttcccaaatgggatacacccggtccgaagctcccctataccccacacctgaacccaacgaaaagggcgagtatcccgagctcggtcttgaggatggaaccgacgcattgaaggacctgtactgctacactggcaagttctacgcccagtgcatggacgagctgctgacccagctgggtattcccacggttaccatcattggacacgactatggtgcctatctgggtcccaagctgtacctatactttccgcaccgggtcgaggccattgccacgtcttgctggcactacgttcctgctctgaagaagttttttgccatccccgactttatcaagctggctccctcattgtcctaccaggcatatcttatcggagatgcttccaaggactttgctacccgagagggcagtgagccgtttctgcgacaagtttttggcggcgaggggtggggcggctcagactcctctgtccatatgactgagcccgagttccagaactacatgaccgagtttgccactggtggtcgctttctgagctacatggtgtccacctacaaggctcgtcgactgacgtttgagatcgaaaagcaggactttttggacaagggccgaacgcccgagagcatgcaagttgatgttccgtatctccatgtcggtgctgagcaggacatggccttccggcctccgatgatcaagaatctgcgaaaatacgtcaagccgggaaggttggacgaggtatggatcgacgctagtcattggctcatgttcgagaaggctgacgaatttaaccacgagctggtcaagtggttggacaagattcatggttcccagtcgaagttgtaa

SEQ ID NO: 16 - DNA, 1047 bp, Yarrowia lipolytica NCYC 3229
atgtcatcactcgacctatcttattacgaaccgttccacagaaatgccgttcttaatggcaaaagatggcactatgtagacattcctggagactccagtggctctcttggtagaggaaagacacttttgctggtccatgggttcccagacacttggtacgggtggcggcatcaagtgcctgtgttccgtaagcacggatttcggctcttgattccctccttgcttggtttcccaagaagtgaagctcctcttactcaccctggagttgccacaggagagaagttcgatggtcacaatgttcacaaggagctgggtctggaagatgaaaacattcaggaacttgaatgttacacggctgacttctttgccaagagcatggtccaactgctggaccaattggggatcgacaaggtctgttcctttggacatgactggggagctgtctttgctccccgactctggctcaattatcctgagagagtcgaatgtgtgtcatcagcatgctggtactaccaaatgccaatggagggcttcgtcgacttgaaagatttcgcagaggtggctccttcattgagataccaattgtactggggaggagacgctcctcaagaagtcatccagaagccgctcaaggaatttatggacagaatgtaccaccatcctacagagaatcatcttctgacatcggaagctgaatacaacaacattctcaaggagtttcaatatggaggaaagaccattgctcccatgtttcctctctacaagtcacgaaaggtagcctacgatatcgatgaaagggactttctgaccaaggggagaacagaggaagacttggcggtggatgtgccgtacttgtttatcggggccgagtttgacattgctcttcagcccggaatggagtctgttttggagggttatgtgaaggagggactactagagaagcaatgggtcccctgtggacattgggtcttgtttgaggagcctgagaagatgaacaagatttatattgactttctgaagaaggtgtttggatcgtcgaagttgtag

SEQ ID NO: 17 - DNA, 1206 bp, Filobasidiella  neoformans var neoformans
atgtcgtattcagaccttccccacaagccgaccattcctgtcgagcccttcaaactatccgtaccccatgaggacctcaatgggctcctcactctactcaaatctaccagaattgccaaagaaagctatgagaatgtctctgcgcacgagaacaaatttggaataacgaggaaatggctggtgaatatgaaagatgaatggatcaaacaggactggcggaaacaagaagagcgaatcaattctcttcctgcgttcaaggcaaaggtaaaaaactcagatggttcggtattttcaatccattttaccgcccttttctcgaaaaagaaggtcgcaatccctatcatcctcagtcacggctggccaggtagcttttacgaattcgttccaatgatggagatggtgaagaagaaatatagtcctgaggatcttccgttccatttaatcgtaccatcccttcccggatggctcttctctacacctcctcctaatgatcgagaattcaatgtgaccgatgttgggtatctcttcaatggtctaatggagggcctaggtttcggagacggctatgttgcccaaggcggtgatattgggagctacgtgacgaacgaacttggtgccaagtacccagcttgcaaaatcattcacgtcaattactctaaccctcctcctcgcccacttccctctccaggctctcctggacaagaggcttcaccgccctctgctgaagatcttcttgagctactgcagaagtttggatatgctctcgagcattctactcgaccagccactgtaggcttggtagtaggctccaatccactcagcttactagcatgggtcggcgagaaattcctcgagtggactgatgagtccccaagtgaggaaacaattttgacaatgacaagcctttattggttcactgattgtttcacgacgtcgatttacacatacagatatggccttggtgctaaacgccatgaaagtgccaaacaagcttcttaccagaaatgtcctctgggctacagccagttccccaaagagatcgtcgagatacctgccgagtgggtcaaggcgcaatccaatatggtttggtctaagaaacatgaatccgggggtcattttgctgctctcgaaaagccagaattgttgtgggcggatatagaggagtttgtacactcgcagtgggagaattacaaaggtaattactga

SEQ ID NO: 18 - DNA, 1185 bp, Rhodotorula mucilaginosa NCYC 3190
atgcccgcccgctcgctcacgctgcgcccgttctcgccgtcgttcacggctccggaactggacggtctcgctcgctcgctcgagtcgtcgcgcttgcccgccgagacgtacgcttcccgccaggccaaatacggcatcaagcatgcttggatgaagaatgccctccaacggtggaaggacgggttcgattggaagaagcacgagcaggacatcaacgaggtcgaccactatatggtgcaggtccagtccgatggcattcaacacgacctccatgtgatctatcacgaatcgaaagacccgaatgcgatcccgctcttgctgctgcatgggtggcccggttccgcgttcgagtttatcgaggcgatcaagatccttcgcaagagtacctcgcccgcgttccacctgatcgcgcccatggagcccggctacgggtggagtactccgccgccactcgaccgcggtttcaacatgaacgattgcacagcgctcatgaacgacttgatggttggactcgggtacggagacggttacgctgctcagggtggcgacatcggttcgggactcgcaagactcctcgccgtcaactatgacgcatgcaaatgcatcaacatcaactacatgcctgccgttgcaccaccagaggacgctccggagcggcaccagatcaaaccgcacgaggaggatgcgctccgacgtgcggacgagtttcagaagacgggcagggggtatgccaacatgcatgcaacgagacccggtacggtcggcatcgtcgtcggtagttcgccggtcgcactgctcgcttggatcgctgaaaagtacctcgcttggaccgatgaggatccgcccctcgacacgatcctcgcaatctgcaccatctggtggatccgcgactcgtacccttcttcaatctgggcctacgccgactttctcgagacgggcatctcggccctgcacaacgacccgaagtacaaacttgacaagaaaccgttcgggttctcgagcttcaaggaggagatcagcgcgactcccgaggcgtgggcgggcaggaacggcaacttgcagttctatcggtaccacgacaagggaggtcactttgcggcgctcgagcagccggaagcgttcgcgcaagacatgcaggattgcttcggcaaaatctggcctctctctcaggagcaaaaatcgtag

SEQ ID NO: 19 - DNA, 16 bp, Artificial Sequence
  Primer
ggagttcttcgcccac

SEQ ID NO: 20 - DNA, 17 bp, Artificial Sequence
  Primer
gatccccaccggaattg

SEQ ID NO: 21 - DNA, 20 bp, Artificial Sequence
  Primer
catacaaccacacacatcca

SEQ ID NO: 22 - DNA, 21 bp, Artificial Sequence
  Primer
taaatagcttagataccacag

SEQ ID NO: 23 - DNA, 19 bp, Artificial Sequence
  Primer
ctctctctccttgtcaact

SEQ ID NO: 24 - DNA, 21 bp, Artificial Sequence
  Primer
gtggatccatggcgacacaca

SEQ ID NO: 25 - DNA, 22 bp, Artificial Sequence
  Primer
gacctaggctacttctcccaca

SEQ ID NO: 26 - DNA, 23 bp, Artificial Sequence
  Primer
gattaatgatcaatgagcgagca

SEQ ID NO: 27 - DNA, 20 bp, Artificial Sequence
  Primer
gacctaggtcacgacgacag

SEQ ID NO: 28 - DNA, 19 bp, Artificial Sequence
  Primer
gtggatccatggctgccca

SEQ ID NO: 29 - DNA, 18 bp, Artificial Sequence
  Primer
gagctagctcaggcctgg

SEQ ID NO: 30 - DNA, 21 bp, Artificial Sequence
  Primer
gtatatctatgcccgcccgct

SEQ ID NO: 31 - DNA, 22 bp, Artificial Sequence
  Primer
gacctaggctacgatttttgct

SEQ ID NO: 32 - DNA, 21 bp, Artificial Sequence
  Primer
gcagatctatgtcatcactcg

SEQ ID NO: 33 - DNA, 22 bp, Artificial Sequence
  Primer
gacctaggctacaacttcgacg

SEQ ID NO: 34 - DNA, 19 bp, Artificial Sequence
  Primer
gtggatccatgatgcaagg

SEQ ID NO: 35 - DNA, 19 bp, Artificial Sequence
  Primer
gacctaggctaaggatatt

SEQ ID NO: 36 - DNA, 22 bp, Artificial Sequence
  Primer
gaggatccatgtcgtattcaga

SEQ ID NO: 37 - DNA, 24 bp, Artificial Sequence
  Primer
gagctagctcagtaattacctttg